(19) United States
(12) Patent Application Publication
(10) Pub. No.: US 2017/0360796 A1
(43) Pub. Date: Dec. 21, 2017

(54) BTK INHIBITOR COMBINATIONS AND DOSING REGIMEN

(71) Applicant: PHARMACYCLICS LLC, Sunnyvale, CA (US)

(72) Inventor: Samantha M. Jaglowski, Columbus, OH (US)

(21) Appl. No.: 15/538,276

(22) PCT Filed: Dec. 22, 2015
(86) PCT No.: PCT/US15/67504

§ 371 Date: Jun. 21, 2017

Related U.S. Application Data
(60) Provisional application No. 62/096,284, filed on Dec. 23, 2014.

(51) Int. Cl. A61K 31/52; A61K 31/675; A61K 31/167; A61K 39/395; A61K 31/196; C07K 16/28; A61K 39/00

(52) U.S. Cl. CPC A61K 31/52; A61K 31/196; A61K 31/675; C07K 16/2887; A61K 31/167; A61K 39/39558; A61K 2039/545; C07K 2317/21; A61K 2039/505

(57) ABSTRACT

Disclosed herein are methods and combination dosing regimen of administering a combination of a BTK inhibitor (e.g. ibrutinib) and an anti-CD20 therapeutic agent for the treatment of a hematologic malignancy. In one aspect is a combination dosing regimen for the treatment of a hematologic malignancy in a subject in need thereof comprising a first phase and a second phase, wherein the first phase is an administration of a BTK inhibitor as a single-agent treatment for a first extended period of time, and the second phase is an administration of a combination of the BTK inhibitor and an anti-CD20 therapeutic agent for a second extended period of time. In one embodiment, the first extended period of time is a period of up to 90 days.

### RELATED APPLICATION

The present application claims the benefit of priority from U.S. Provisional Patent Application No. 62/096,284, filed Dec. 23, 2014, which is herein incorporated by reference in its entirety.

### BACKGROUND

Bruton's tyrosine kinase (BTK), a member of the Tec family of non-receptor tyrosine kinases, is a key signaling enzyme expressed in all hematopoietic cells types except T lymphocytes and natural killer cells. Btk plays an essential role in the B-cell signaling pathway linking cell surface B-cell receptor (BCR) stimulation to downstream intracellular responses.

1-((R)-3-(4-amino-3-(4-phenoxyphenyl)-1H-pyrazolo[3,4-d]pyrimidin-1-yl)piperidin-1-yl)prop-2-en-1-one is also known by its IUPAC name as 1-{(3R)-3-[4-amino-3-(4-phenoxyphenyl)-1H-pyrazolo[3,4-d]pyrimidin-1-yl]piperidin-1-yl}prop-2-en-1-one or 2-Propen-1-one, 1-[(3R)-3-[4-amino-3-(4-phenoxyphenyl)-1H-pyrazolo[3,4-d]pyrimidin-1-yl]-1-piperidinyl-, and has been given the USAN name, ibrutinib. The various names given for ibrutinib are used interchangeably herein.

### SUMMARY OF THE INVENTION

In one aspect is a combination dosing regimen for the treatment of a hematologic malignancy in a subject in need thereof comprising a first phase and a second phase, wherein the first phase is an administration of a BTK inhibitor as a single-agent treatment for a first extended period of time, and the second phase is an administration of a combination of the BTK inhibitor and an anti-CD20 therapeutic agent for a second extended period of time. In one embodiment, the first extended period of time is a period of up to 90 days. In another embodiment, is a combination dosing regimen, wherein the first extended period of time is a period of up to 60 days. In a further embodiment, is a combination dosing regimen, wherein the first extended period of time is a period of up to 28 days.

In another embodiment, is a combination dosing regimen, wherein the first extended period of time is a period of up to 14 days. In another embodiment, is a combination dosing regimen, wherein the second extended period of time is a period of up to 40 weeks. In yet another embodiment, the second extended period of time is a period of up to 35 weeks. In yet a further embodiment, the second extended period of time is a period of up to 30 weeks. In another embodiment, is a combination dosing regimen, wherein the second extended period of time is a period of up to 25 weeks. In yet another embodiment, is a combination dosing regimen, wherein the BTK inhibitor and the anti-CD20 therapeutic agent for a period of up to 52 weeks. In a further embodiment, is a combination dosing regimen, wherein the combination dosing regimen is an administration of the BTK inhibitor and the anti-CD20 therapeutic agent for a period of up to 37 weeks. In another embodiment, is a combination dosing regimen, wherein the combination dosing regimen is an administration of the BTK inhibitor and the anti-CD20 therapeutic agent for a period of up to 29 weeks. In another embodiment, is a combination dosing regimen, wherein the combination dosing regimen is an administration of the BTK inhibitor and the anti-CD20 therapeutic agent for a period of up to 27 weeks. In one embodiment, is a combination dosing regimen, wherein the combination dosing regimen is an administration of the BTK inhibitor and the anti-CD20 therapeutic agent for a period of up to 25 weeks. In a further embodiment is a combination dosing regimen, wherein the anti-CD20 therapeutic agent comprises ofatumumab, rituximab, obinutuzumab, ibritumomab tiuxetan, tositumomab, FBTA05, iodine I 131/tositumomab, obinutuzumab, ocaratuzumab (AME-133v), ocrelizumab, TRU-015, veltuzumab (IMMU-106), or a combination thereof.

In one embodiment, is a combination dosing regimen wherein the anti-CD20 therapeutic agent is ofatumumab. In some embodiments, ofatumumab is administered intravenously. In a further embodiment, is a combination dosing regimen, wherein ofatumumab is administered at most 12 infusions during the course of the therapy treatment. In a further embodiment, is a combination dosing regimen wherein ofatumumab is administered at a dosage of about 300 mg/day to about 2000 mg/day. In a further embodiment, is a combination dosing regimen wherein the BTK inhibitor is ibrutinib. In another embodiment, is a combination dosing regimen wherein ibrutinib is administered orally. In a further embodiment, is a combination dosing regimen wherein ibrutinib is administered once a day, two times per day, three times per day, four times per day, or five times per day. In one embodiment, is a combination dosing regimen wherein ibrutinib is administered once a day. In yet a further embodiment, ibrutinib is administered at a dosage of about 40 mg/day to about 1000 mg/day. In a further embodiment, is a combination dosing regimen wherein ibrutinib is administered at a dosage of about 100 mg/day to about 900 mg/day. In another embodiment, is a combination dosing regimen wherein ibrutinib is administered at a dosage of about 420 mg/day to about 840 mg/day. In one embodiment, is a combination dosing regimen wherein ibrutinib is administered at a dosage of about 420 mg/day. In another embodiment, is a combination dosing regimen wherein the hematologic malignancy is a leukemia, a lymphoma, a myeloma, a non-Hodgkin's lymphoma, a Hodgkin's lymphoma, a T-cell malignancy, or a B-cell malignancy. In a further embodiment, is a combination dosing regimen wherein the hematologic malignancy is a B-cell malignancy. In a further embodiment, is a combination dosing regimen wherein the B-cell malignancy is chronic lymphocytic leukemia (CLL), small lymphocytic lymphoma (SLL), high risk CLL, non-CLL/SLL lymphoma, prolymphocytic leukemia (PLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In yet a further embodiment, the B-cell malignancy is CLL. In a further embodiment, is a combination dosing regimen wherein the B-cell malignancy is SLL. In yet another embodiment, the B-cell malignancy is PLL. In a further embodiment, is a combination dosing regimen wherein the B-cell malignancy is DLBCL. In another embodiment, is a combination dosing regimen wherein the B-cell malignancy is MCL. In a further embodiment, is a combination dosing regimen wherein the B-cell malignancy is Waldenström's macroglobulinemia. In a further embodiment, is a combination dosing regimen wherein the hematologic malignancy is a relapsed or refractory hematologic malignancy. In yet a further embodiment, the hematologic malignancy is a metastasized hematologic malignancy.

In one embodiment, is a combination dosing regimen wherein the combination dosing regimen further comprises administration of an additional therapeutic agent. In another embodiment, is a combination dosing regimen wherein the additional therapeutic agent is selected from among an analgesic, an antihistamine, a chemotherapeutic agent, or a radiation therapeutic agent. In yet another embodiment, is a combination dosing regimen wherein the analgesic is acetaminophen. In a further embodiment, is a combination dosing regimen wherein the antihistamine is cetirizen. In yet a further embodiment, is a combination dosing regimen wherein the chemotherapeutic agent is selected from among chlorambucil, ifosfamide, doxorubicin, mesalazine, thalidomide, lenalidomide, temsirolimus, everolimus, fludarabine, fostamatinib, paclitaxel, docetaxel, dexamethasone, prednisone, CAL-101, ibritumomab, tositumomab, bortezomib, pentostatin, endostatin, or a combination thereof.

In one aspect, is a combination dosing regimen for the treatment of a hematologic malignancy in a subject in need thereof comprising a first phase and a second phase, wherein the first phase is an administration of ibrutinib as a single-agent treatment for a first extended period of time, and the second phase is an administration of a combination of ibrutinib and an anti-CD20 therapeutic agent for a second extended period of time.

In one embodiment, is a combination dosing regimen for the treatment of chronic lymphocytic leukemia in a subject in need thereof comprising a first phase and a second phase, wherein the first phase is an administration of a BTK inhibitor as a single-agent treatment for a first extended period of time, and the second phase is an administration of a combination of the BTK inhibitor and an anti-CD20 therapeutic agent for a second extended period of time.

In a further embodiment, is a combination dosing regimen for the treatment of chronic lymphocytic leukemia in a subject in need thereof comprising a first phase and a second phase, wherein the first phase is an administration of ibrutinib as a single-agent treatment for a first extended period of time, and the second phase is an administration of a combination of ibrutinib and an anti-CD20 therapeutic agent for a second extended period of time.

In one aspect is a method of treating a hematologic malignancy in a subject in need thereof, comprising administering to the subject a therapeutically effective amount of a combination comprising a BTK inhibitor and an anti-CD20 therapeutic agent following a combination dosing regimen wherein the combination dosing regimen comprises administering the BTK inhibitor as a single-agent over a first extended period of time as a first phase prior to administering the combination of the BTK inhibitor and the anti-CD20 therapeutic agent over a second extended period of time as a second phase.

In one embodiment, is a method of treating a hematologic malignancy in a subject in need thereof comprising administering to the subject a therapeutically effective amount of a combination comprising a BTK inhibitor and an anti-CD20 therapeutic agent following a combination dosing regimen wherein the first extended period of time is a period of up to 90 days as a first phase. In one embodiment, the first extended period of time is a period of up to 60 days as a first phase. In another embodiment, the first extended period of time is a period of up to 28 days as a first phase. In a further embodiment, the first extended period of time is a period of up to 14 days as a first phase. In yet a further embodiment, the second extended period of time is a period of up to 40 weeks. In one embodiment, the second extended period of time is a period of up to 35 weeks. In another embodiment, the second extended period of time is a period of up to 30 weeks. In yet another embodiment, the second extended period of time is a period of up to 25 weeks. In a further embodiment, the combination dosing regimen is administered for a period of up to 52 weeks. In yet a further embodiment, the combination dosing regimen is administered for a period of up to 37 weeks. In one embodiment, the combination dosing regimen is administered for a period of up to 29 weeks. In another embodiment, the combination dosing regimen is administered for a period of up to 27 weeks. In yet another embodiment, the combination dosing regimen is administered for a period of up to 25 weeks. In a further embodiment, the anti-CD20 therapeutic agent comprises ofatumumab, rituximab, obinutuzumab, ibritumomab tiuxetan, tositumomab, FBTA05, iodine I 131/tositumomab, obinutuzumab, ocaratuzumab (AME-133v), ocrelizumab, TRU-015, veltuzumab (IMMU-106), or a combination thereof. In one embodiment, the anti-CD20 therapeutic agent is ofatumumab. In another embodiment, ofatumumab is administered intravenously. In yet another embodiment, ofatumumab is administered at most 12 infusions during the course of the therapy treatment. In one embodiment, ofatumumab is administered at a dosage of about 300 mg/day to about 2000 mg/day. In a further embodiment, the BTK inhibitor is ibrutinib. In yet a further embodiment, ibrutinib is administered orally. In one embodiment, ibrutinib is administered once a day, two times per day, three times per day, four times per day, or five times per day. In another embodiment, ibrutinib is administered once a day. In one embodiment, ibrutinib is administered at a dosage of about 40 mg/day to about 1000 mg/day. In one embodiment, ibrutinib is administered at a dosage of about 100 mg/day to about 900 mg/day. In another embodiment, ibrutinib is administered at a dosage of about 420 mg/day to about 840 mg/day. In another embodiment, ibrutinib is administered at a dosage of about 420 mg/day. In yet another embodiment, the hematologic malignancy is a leukemia, a lymphoma, a myeloma, a non-Hodgkin's lymphoma, a Hodgkin's lymphoma, a T-cell malignancy, or a B-cell malignancy. In one embodiment, the hematologic malignancy is a B-cell malignancy. In one embodiment, the B-cell malignancy is chronic lymphocytic leukemia (CLL), small lymphocytic lymphoma (SLL), high risk CLL, non-CLL/SLL lymphoma, prolymphocytic leukemia (PLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In one embodiment, the B-cell malignancy is CLL.

In one embodiment, the B-cell malignancy is SLL. In one embodiment, the B-cell malignancy is PLL. In one embodiment, the B-cell malignancy is DLBCL. In one embodiment, the B-cell malignancy is MCL. In one embodiment, the B-cell malignancy is Waldenström's macroglobulinemia. In one embodiment, the hematologic malignancy is a relapsed or refractory hematologic malignancy. In one embodiment, the hematologic malignancy is a metastasized hematologic malignancy. In one embodiment, the method further comprises administering an additional therapeutic agent. In one embodiment, the additional therapeutic agent is selected from among an analgesic, an antihistamine, a chemotherapeutic agent, or a radiation therapeutic agent.

In one embodiment, the analgesic is acetaminophen. In one embodiment, the antihistamine is cetirizen. In one embodiment, the chemotherapeutic agent is selected from among chlorambucil, ifosfamide, doxorubicin, mesalazine, thalidomide, lenalidomide, temsirolimus, everolimus, fludarabine, fostamatinib, paclitaxel, docetaxel, dexamethasone, prednisone, CAL-101, ibritumomab, tositumomab, bortezomib, pentostatin, endostatin, or a combination thereof. In another embodiment, the combination regimen leads to extension of disease remission. In one embodiment, the combination regimen leads to a decrease of disease progression.

In one aspect is a method of treating a hematologic malignancy in a subject in need thereof, comprising administering to the subject a therapeutically effective amount of a combination comprising ibrutinib and an anti-CD20 therapeutic agent following a combination dosing regimen wherein the combination dosing regimen comprises administering ibrutinib as a single-agent over a first extended period of time as a first phase prior to administering the combination of ibrutinib and the anti-CD20 therapeutic agent over a second extended period of time as a second phase.

In a further aspect is a method of treating chronic lymphocytic leukemia in a subject in need thereof, comprising administering to the subject a therapeutically effective amount of a combination comprising a BTK inhibitor and an anti-CD20 therapeutic agent following a combination dosing regimen wherein the combination dosing regimen comprises administering the BTK inhibitor as a single-agent over a first extended period of time as a first phase prior to administering the combination of the BTK inhibitor and the anti-CD20 therapeutic agent over a second extended period of time as a second phase.

In a further aspect is a method of treating chronic lymphocytic leukemia in a subject in need thereof, comprising administering to the subject a therapeutically effective amount of a combination comprising ibrutinib and an anti-CD20 therapeutic agent following a combination dosing regimen wherein the combination dosing regimen comprises administering ibrutinib as a single-agent over a first extended period of time as a first phase prior to administering the combination of ibrutinib and the anti-CD20 therapeutic agent over a second extended period of time as a second phase.

### BRIEF DESCRIPTION OF THE DRAWINGS

Various aspects of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates treatment schema by group. Oral ibrutinib 420 mg was given once daily until disease progression or unacceptable toxicity. IV ofatumumab was given as 8 weekly infusions followed by 4 monthly infusions for a total of 12 doses (dose 1, 300 mg; doses 2-12, 2000 mg). Group 1: ibrutinib monotherapy during cycle 1, then ofatumumab added starting cycle 2. Group 2: ofatumumab and ibrutinib starting on days 1 and 2 of cycle 1, respectively. Group 3: ofatumumab monotherapy for the first 2 cycles, then ibrutinib added starting cycle 3.

FIG. 2 illustrates outcomes with study treatment. (A) Best response among CLL/SLL patients by group. CR, complete response; PD, progressive disease; PR, partial response; PR-L, partial response with lymphocytosis; SD, stable disease. The asterisk (*) indicates that 4 patients (17%) in group 3 developed PD while receiving ofatumumab monotherapy; (B) Forest plot of response rates by patient subgroups. (C) Median percent change in ALC from baseline by group; (D-F) Median percent change in the sum of the products of lymph node diameters (SPD) and absolute lymphocyte count (ALC) by group.

FIG. 3 shows Kaplan-Meier curves of progression-free survival by group.

### DETAILED DESCRIPTION OF THE INVENTION

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

The section headings used herein are for organizational purposes only and are not to be construed as limiting the subject matter described.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

### Overview

Disclosed herein are methods and combination dosing regimens that comprise a combination of a TEC inhibitor and an anti-CD20 therapeutic agent. Also described are methods of administrating a combination of a TEC inhibitor and an anti-CD20 therapeutic agent for the treatment of a hematologic malignancy. In some instances, the TEC inhibitor is a BTK, ITK, TEC, RLK, or BMX inhibitor. In some instances, the TEC inhibitor is an ITK inhibitor. In some instances, the TEC inhibitor is a BTK inhibitor.

In some instances, provided herein is a combination dosing regimen for the treatment of a hematologic malignancy in a subject in need thereof comprising a first phase and a second phase, wherein the first phase is an administration of a BTK inhibitor as a single-agent treatment for a first extended period of time, and the second phase is an administration of a combination of the BTK inhibitor and an anti-CD20 therapeutic agent for a second extended period of time.

In some instances, provided herein is a combination dosing regimen for the treatment of a hematologic malignancy in a subject in need thereof comprising a first phase and a second phase, wherein the first phase is an administration of ibrutinib as a single-agent treatment for a first extended period of time, and the second phase is an administration of a combination of ibrutinib and an anti-CD20 therapeutic agent for a second extended period of time.

In some instances, provided herein is a combination dosing regimen for the treatment of chronic lymphocytic leukemia in a subject in need thereof comprising a first phase and a second phase, wherein the first phase is an administration of a BTK inhibitor as a single-agent treatment for a first extended period of time, and the second phase is an administration of a combination of the BTK inhibitor and an anti-CD20 therapeutic agent for a second extended period of time.

In some instances, provided herein is a combination dosing regimen for the treatment of chronic lymphocytic leukemia in a subject in need thereof comprising a first phase and a second phase, wherein the first phase is an administration of ibrutinib as a single-agent treatment for a first extended period of time, and the second phase is an administration of a combination of ibrutinib and an anti-CD20 therapeutic agent for a second extended period of time.

In some cases, provided herein is a method of treating a hematologic malignancy in a subject in need thereof, comprising administering to the subject a therapeutically effective amount of a combination comprising a BTK inhibitor and an anti-CD20 therapeutic agent following a combination dosing regimen wherein the combination dosing regimen comprises administering the BTK inhibitor as a single-agent over a first extended period of time as a first phase prior to administering the combination of the BTK inhibitor and the anti-CD20 therapeutic agent over a second extended period of time as a second phase.

In some aspects, provided herein is a method of treating a hematologic malignancy in a subject in need thereof, comprising administering to the subject a therapeutically effective amount of a combination comprising ibrutinib and an anti-CD20 therapeutic agent following a combination dosing regimen wherein the combination dosing regimen comprises administering ibrutinib as a single-agent over a first extended period of time as a first phase prior to administering the combination of ibrutinib and the anti-CD20 therapeutic agent over a second extended period of time as a second phase.

In some cases, provided herein is a method of treating chronic lymphocytic leukemia in a subject in need thereof, comprising administering to the subject a therapeutically effective amount of a combination comprising a BTK inhibitor and an anti-CD20 therapeutic agent following a combination dosing regimen wherein the combination dosing regimen comprises administering the BTK inhibitor as a single-agent over a first extended period of time as a first phase prior to administering the combination of the BTK inhibitor and the anti-CD20 therapeutic agent over a second extended period of time as a second phase.

In some embodiments, provided herein is a method of treating chronic lymphocytic leukemia in a subject in need thereof, comprising administering to the subject a therapeutically effective amount of a combination comprising ibrutinib and an anti-CD20 therapeutic agent following a combination dosing regimen wherein the combination dosing regimen comprises administering ibrutinib as a single-agent over a first extended period of time as a first phase prior to administering the combination of ibrutinib and the anti-CD20 therapeutic agent over a second extended period of time as a second phase.

### Anti-CD20 Therapeutic Agents

In some embodiments, an anti-CD20 therapeutic agent is an antibody. In some instances, the antibody is a monoclonal antibody. In some instances, the anti-CD20 therapeutic agent is an anti-CD20 monoclonal antibody.

Exemplary anti-CD20 therapeutic agent comprises rituximab (Rituxan®), ofatumumab (Arzerra®), obinutuzumab, ibritumomab tiuxetan (In-111 Zevalin®, Y-90 Zevalin®, Zevalin®), tositumomab (Bexxar Therapeutic, Bexxar Dosimetric), FBTA05, iodine I 131/tositumomab (Bexxar), obinutuzumab (Gazyva®), ocaratuzumab (AME-133v), ocrelizumab, TRU-015, or veltuzumab (IMMU-106).

In some instances, the anti-CD20 therapeutic agent is an anti-CD20 therapeutic agent as described in U.S. Pat. No. 8,101,179, U.S. Pat. No. 8,057,793, US20130089540, US20100303808, US20090060921, US20090203886, or US20050180972.

In some cases, described herein is a combination dosing regimen for the treatment of a hematologic malignancy in a subject in need thereof comprising a combination of a TEC inhibitor and an anti-CD20 therapeutic agent selected from rituximab (Rituxan®), ofatumumab (Arzerra®), obinutuzumab, ibritumomab tiuxetan (In-111 Zevalin®, Y-90 Zevalin®, Zevalin®), tositumomab (Bexxar Therapeutic, Bexxar Dosimetric), FBTA05, iodine I 131/tositumomab (Bexxar), obinutuzumab (Gazyva®), ocaratuzumab (AME-133v), ocrelizumab, TRU-015, veltuzumab (IMMU-106), or a combination thereof. In some instances, the TEC inhibitor is an ITK inhibitor or a BTK inhibitor.

In some cases, described herein is a combination dosing regimen for the treatment of a hematologic malignancy in a subject in need thereof comprising a combination of an ITK inhibitor and an anti-CD20 therapeutic agent selected from rituximab (Rituxan®), ofatumumab (Arzerra®), obinutuzumab, ibritumomab tiuxetan (In-111 Zevalin®, Y-90 Zevalin®, Zevalin®), tositumomab (Bexxar Therapeutic, Bexxar Dosimetric), FBTA05, iodine I 131/tositumomab (Bexxar), obinutuzumab (Gazyva®), ocaratuzumab (AME-133v), ocrelizumab, TRU-015, veltuzumab (IMMU-106), or a combination thereof.

In some cases, described herein is a combination dosing regimen for the treatment of a hematologic malignancy in a subject in need thereof comprising a combination of a BTK inhibitor and an anti-CD20 therapeutic agent selected from rituximab (Rituxan®), ofatumumab (Arzerra®), obinutuzumab, ibritumomab tiuxetan (In-111 Zevalin®, Y-90 Zevalin®, Zevalin®), tositumomab (Bexxar Therapeutic, Bexxar Dosimetric), FBTA05, iodine I 131/tositumomab (Bexxar), obinutuzumab (Gazyva®), ocaratuzumab (AME-133v), ocrelizumab, TRU-015, veltuzumab (IMMU-106), or a combination thereof. In some instances, the BTK inhibitor is selected from ibrutinib, PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) or LFM-A13. In some embodiments, the BTK inhibitor is ibrutinib.

In some cases, described herein is a combination dosing regimen for the treatment of a hematologic malignancy in a subject in need thereof comprising a combination of ibrutinib and an anti-CD20 therapeutic agent selected from rituximab (Rituxan®), ofatumumab (Arzerra®), obinutuzumab, ibritumomab tiuxetan (In-111 Zevalin®, Y-90 Zevalin®, Zevalin®), tositumomab (Bexxar Therapeutic, Bexxar Dosimetric), FBTA05, iodine I 131/tositumomab (Bexxar), obinutuzumab (Gazyva®), ocaratuzumab (AME-133v), ocrelizumab, TRU-015, veltuzumab (IMMU-106), or a combination thereof. In some instances, the anti-CD20 therapeutic agent is ofatumumab.

In some cases, described herein is a combination dosing regimen for the treatment of a hematologic malignancy in a subject in need thereof comprising a combination of ibrutinib and ofatumumab.

Also described herein is a method of treating a hematologic malignancy in a subject in need thereof, that comprises administering to the subject a therapeutically effective amount of a combination comprising a TEC inhibitor and an anti-CD20 therapeutic agent selected from rituximab (Rituxan®), ofatumumab (Arzerra®), obinutuzumab, ibritumomab tiuxetan (In-111 Zevalin®, Y-90 Zevalin®, Zevalin®), tositumomab (Bexxar Therapeutic, Bexxar Dosimetric), FBTA05, iodine I 131/tositumomab (Bexxar), obinutuzumab (Gazyva®), ocaratuzumab (AME-133v), ocrelizumab, TRU-015, veltuzumab (IMMU-106), or a combination thereof, following a combination dosing regimen. In some instances, the TEC inhibitor is an ITK inhibitor or a BTK inhibitor.

Also described herein is a method of treating a hematologic malignancy in a subject in need thereof, that comprises administering to the subject a therapeutically effective amount of a combination comprising an ITK inhibitor and an anti-CD20 therapeutic agent selected from rituximab (Rituxan®), ofatumumab (Arzerra®), obinutuzumab, ibritumomab tiuxetan (In-111 Zevalin®, Y-90 Zevalin®, Zevalin®), tositumomab (Bexxar Therapeutic, Bexxar Dosimetric), FBTA05, iodine I 131/tositumomab (Bexxar), obinutuzumab (Gazyva®), ocaratuzumab (AME-133v), ocrelizumab, TRU-015, veltuzumab (IMMU-106), or a combination thereof, following a combination dosing regimen.

Also described herein is a method of treating a hematologic malignancy in a subject in need thereof, that comprises administering to the subject a therapeutically effective amount of a combination comprising a BTK inhibitor and an anti-CD20 therapeutic agent selected from rituximab (Rituxan®), ofatumumab (Arzerra®), obinutuzumab, ibritumomab tiuxetan (In-111 Zevalin®, Y-90 Zevalin®, Zevalin®), tositumomab (Bexxar Therapeutic, Bexxar Dosimetric), FBTA05, iodine I 131/tositumomab (Bexxar), obinutuzumab (Gazyva®), ocaratuzumab (AME-133v), ocrelizumab, TRU-015, veltuzumab (IMMU-106), or a combination thereof, following a combination dosing regimen. In some instances, the BTK inhibitor is selected from ibrutinib, PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) or LFM-A13. In some embodiments, the BTK inhibitor is ibrutinib.

Also described herein is a method of treating a hematologic malignancy in a subject in need thereof, that comprises administering to the subject a therapeutically effective amount of a combination comprising ibrutinib and an anti-CD20 therapeutic agent selected from rituximab (Rituxan®), ofatumumab (Arzerra®), obinutuzumab, ibritumomab tiuxetan (In-111 Zevalin®, Y-90 Zevalin®, Zevalin®), tositumomab (Bexxar Therapeutic, Bexxar Dosimetric), FBTA05, iodine I 131/tositumomab (Bexxar), obinutuzumab (Gazyva®), ocaratuzumab (AME-133v), ocrelizumab, TRU-015, veltuzumab (IMMU-106), or a combination thereof, following a combination dosing regimen. In some instances, the anti-CD20 therapeutic agent is ofatumumab.

Also described herein is a method of treating a hematologic malignancy in a subject in need thereof, that comprises administering to the subject a therapeutically effective amount of a combination comprising ibrutinib and ofatumumab following a combination dosing regimen.

As used herein, the term “antibody” is used in the broadest sense and covers fully assembled antibodies, antibody fragments that can bind antigen (e.g., Fab, F(ab′)₂, Fv, single chain antibodies, diabodies, antibody chimeras, hybrid antibodies, bispecific antibodies, humanized antibodies, and the like), and recombinant peptides comprising the forgoing.

The terms “monoclonal antibody” and “mAb” as used herein refer to an antibody obtained from a substantially homogeneous population of antibodies, i.e., the individual antibodies comprising the population are identical except for possible naturally occurring mutations that may be present in minor amounts.

In some instances, antibodies are heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy-chain variable domains.

The term “variable” refers to the fact that certain portions of the variable domains differ extensively in sequence among antibodies. Variable regions confer antigen-binding specificity. However, the variability is not evenly distributed throughout the variable domains of antibodies. It is concentrated in three segments called complementarity determining regions (CDRs) or hypervariable regions, both in the light chain and the heavy-chain variable domains. The more highly conserved portions of variable domains are celled in the framework (FR) regions. The variable domains of native heavy and light chains each comprise four FR regions, largely adopting a β-pleated-sheet configuration, connected by three CDRs, which form loops connecting, and in some cases forming part of, the β-pleated-sheet structure. The CDRs in each chain are held together in close proximity by the FR regions and, with the CDRs from the other chain, contribute to the formation of the antigen-binding site of antibodies (see, Kabat et al. (1991) NIH PubL. No. 91-3242, Vol. I, pages 647-669). The constant domains are not involved directly in binding an antibody to an antigen, but exhibit various effector functions, such as Fc receptor (FcR) binding, participation of the antibody in antibody-dependent cellular toxicity, initiation of complement dependent cytotoxicity, and mast cell degranulation.

The term “hypervariable region,” when used herein, refers to the amino acid residues of an antibody that are responsible for antigen-binding. The hypervariable region comprises amino acid residues from a “complementarily determining region” or “CDR” (i.e., residues 24-34 (L1), 50-56 (L2), and 89-97 (L3) in the light-chain variable domain and 31-35 (H1), 50-65 (H2), and 95-102 (H3) in the heavy-chain variable domain; Kabat et al. (1991) Sequences of Proteins of Immunological Interest, 5th Ed. Public Health Service, National Institute of Health, Bethesda, Md.) and/or those residues from a “hypervariable loop” (i.e., residues 26-32 (L1), 50-52 (L2), and 91-96 (L3) in the light-chain variable domain and (H1), 53-55 (H2), and 96-101 (13) in the heavy chain variable domain; Clothia and Lesk, (1987) J. Mol. Biol., 196:901-917). “Framework” or “FR” residues are those variable domain residues other than the hypervariable region residues, as herein deemed.

“Antibody fragments” comprise a portion of an intact antibody, preferably the antigen-binding or variable region of the intact antibody. Examples of antibody fragments include Fab, Fab, F(ab′)2, and Fv fragments; diabodies; linear antibodies (Zapata et al. (1995) Protein Eng. 10:1057-1062); single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen.

“Fv” is the minimum antibody fragment that contains a complete antigen recognition and binding site. This region consists of a dimer of one heavy- and one light-chain variable domain in tight, non-covalent association. It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (C_{H1}) of the heavy chain. Fab fragments differ from Fab′ fragments by the addition of a few residues at the carboxy terminus of the heavy chain C_{H1} domain including one or more cysteines from the antibody hinge region. Fab′-SH is the designation herein for Fab′ in which the cysteine residue(s) of the constant domains bear a free thiol group. Fab′ fragments are produced by reducing the F(ab′)2 fragment's heavy chain disulfide bridge. Other chemical couplings of antibody fragments are also known.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequence of the constant domain of their heavy chains, immunoglobulins can be assigned to different classes. There are five major classes of human immunoglobulins: IgA, IgD, IgE, IgG, and IgM, and several of these may be further divided into subclasses (isotypes), e.g., IgG1, IgG2, IgG3, IgG4, IgA1, and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known. Different isotypes have different effector functions. For example, human IgG1 and IgG3 isotypes have ADCC (antibody dependent cell-mediated cytotoxicity) activity.

### Combination Dosing Regimen

In some instances, the combination dosing regimen comprises a first phase and a second phase. In some instances, the first phase comprises administration of a TEC inhibitor as a single-agent treatment for a first extended period of time prior to administration of the second phase for a second extended period of time. In some instances, the second phase comprises administration of a combination a TEC inhibitor and an anti-CD20 therapeutic agent. In some instances, the TEC inhibitor is an ITK inhibitor. In some instances, the TEC inhibitor is a BTK inhibitor.

In some instances, the first phase comprises administration of an ITK inhibitor as a single-agent treatment for a first extended period of time prior to administration of the second phase for a second extended period of time. In some instances, the second phase comprises administration of a combination an ITK inhibitor and an anti-CD20 therapeutic agent.

In some instances, the first phase comprises administration of a BTK inhibitor as a single-agent treatment for a first extended period prior to administration of the second phase for a second extended period of time. In some instances, the second phase comprises administration of a combination a BTK inhibitor and an anti-CD20 therapeutic agent. In some embodiments, the BTK inhibitor is selected from ibrutinib, PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) or LFM-A13. In some embodiments, the BTK inhibitor is ibrutinib.

In some instances, the first phase comprises administration of ibrutinib as a single-agent treatment for a first extended period prior to administration of the second phase for a second extended period of time. In some instances, the second phase comprises administration of a combination ibrutinib and an anti-CD20 therapeutic agent.

In some instances, the first extended period of time is a period of up to 90 days. In some instances, the first extended period of time is a period of up to 85, 80, 75, 70, 65, 60, 55, 50, 45, 40, 39, 38, 37, 36, 35, 34, 33, 32, 31, 30, 29, 28, 27, 26, 25, 24, 23, 22, 21, 20, 19, 18, 17, 16, 15, 10, or 5 days. In some embodiments, the first extended period of time is a period of up to 60 days. In some embodiments, the first extended period of time is a period of up to 28 days. In some embodiments, the first extended period of time is a period of up to 14 days.

In some embodiments, the first phase comprises administration of a TEC inhibitor as a single-agent treatment for a period of up to 90, 85, 80, 75, 70, 65, 60, 55, 50, 45, 40, 39, 38, 37, 36, 35, 34, 33, 32, 31, 30, 29, 28, 27, 26, 25, 24, 23, 22, 21, 20, 19, 18, 17, 16, 15, 10, or 5 days. In some instances, the TEC inhibitor is an ITK inhibitor or a BTK inhibitor.

In some embodiments, the first phase comprises administration of an ITK inhibitor as a single-agent treatment for a period of up to 90, 85, 80, 75, 70, 65, 60, 55, 50, 45, 40, 39, 38, 37, 36, 35, 34, 33, 32, 31, 30, 29, 28, 27, 26, 25, 24, 23, 22, 21, 20, 19, 18, 17, 16, 15, 10, or 5 days. In some embodiments, the first phase comprises administration of an ITK inhibitor as a single-agent treatment for a period of up to 90 days. In some embodiments, the first phase comprises administration of an ITK inhibitor as a single-agent treatment for a period of up to 60 days. In some embodiments, the first phase comprises administration of an ITK inhibitor as a single-agent treatment for a period of up to 28 days. In some embodiments, the first phase comprises administration of an ITK inhibitor as a single-agent treatment for a period of up to 14 days.

In some embodiments, the first phase comprises administration of a BTK inhibitor as a single-agent treatment for a period of up to 90, 85, 80, 75, 70, 65, 60, 55, 50, 45, 40, 39, 38, 37, 36, 35, 34, 33, 32, 31, 30, 29, 28, 27, 26, 25, 24, 23, 22, 21, 20, 19, 18, 17, 16, 15, 10, or 5 days. In some embodiments, the first phase comprises administration of a BTK inhibitor as a single-agent treatment for a period of up to 90 days. In some embodiments, the first phase comprises administration of a BTK inhibitor as a single-agent treatment for a period of up to 60 days. In some embodiments, the first phase comprises administration of a BTK inhibitor as a single-agent treatment for a period of up to 28 days. In some embodiments, the first phase comprises administration of a BTK inhibitor as a single-agent treatment for a period of up to 14 days. In some embodiments, the BTK inhibitor is selected from ibrutinib, PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) or LFM-A13. In some embodiments, the BTK inhibitor is ibrutinib.

In some embodiments, the first phase comprises administration of ibrutinib as a single-agent treatment for a period of up to 90, 85, 80, 75, 70, 65, 60, 55, 50, 45, 40, 39, 38, 37, 36, 35, 34, 33, 32, 31, 30, 29, 28, 27, 26, 25, 24, 23, 22, 21, 20, 19, 18, 17, 16, 15, 10, or 5 days. In some embodiments, the first phase comprises administration of ibrutinib as a single-agent treatment for a period of up to 90 days. In some embodiments, the first phase comprises administration of ibrutinib as a single-agent treatment for a period of up to 60 days. In some embodiments, the first phase comprises administration of ibrutinib as a single-agent treatment for a period of up to 28 days. In some embodiments, the first phase comprises administration of ibrutinib as a single-agent treatment for a period of up to 14 days.

In some embodiments, the second extended period of time is a period of up to 40 weeks. In some cases, the second extended period of time is a period of up to 35, 30, 25, 20, or 15 weeks. In some embodiments, the second extended period of time is a period of up to 35 weeks. In some embodiments, the second extended period of time is a period of up to 30 weeks. In some embodiments, the second extended period of time is a period of up to 25 weeks.

In some embodiments, the second phase comprises administration of a combination of a TEC inhibitor and an anti-CD20 therapeutic agent for a period of up to 40, 35, 30, 25, 20, or 15 weeks. In some instances, the TEC inhibitor is an ITK inhibitor or a BTK inhibitor. In some instances, the anti-CD20 therapeutic agent is selected from ofatumumab, rituximab, obinutuzumab, ibritumomab tiuxetan, tositumomab, FBTA05, iodine I 131/tositumomab, obinutuzumab, ocaratuzumab (AME-133v), ocrelizumab, TRU-015, veltuzumab (IMMU-106), or a combination thereof.

In some embodiments, the second phase comprises administration of a combination of an ITK inhibitor and an anti-CD20 therapeutic agent for a period of up to 40, 35, 30, 25, 20, or 15 weeks. In some embodiments, the second phase comprises administration of a combination of an ITK inhibitor and an anti-CD20 therapeutic agent for a period of up to 40 weeks. In some embodiments, the second phase comprises administration of a combination of an ITK inhibitor and an anti-CD20 therapeutic agent for a period of up to 35 weeks. In some embodiments, the second phase comprises administration of a combination of an ITK inhibitor and an anti-CD20 therapeutic agent for a period of up to 30 weeks. In some embodiments, the second phase comprises administration of a combination of an ITK inhibitor and an anti-CD20 therapeutic agent for a period of up to 25 weeks. In some instances, the anti-CD20 therapeutic agent is selected from ofatumumab, rituximab, obinutuzumab, ibritumomab tiuxetan, tositumomab, FBTA05, iodine I 131/tositumomab, obinutuzumab, ocaratuzumab (AME-133v), ocrelizumab, TRU-015, veltuzumab (IMMU-106), or a combination thereof.

In some embodiments, the second phase comprises administration of a combination of a BTK inhibitor and an anti-CD20 therapeutic agent for a period of up to 40, 35, 30, 25, 20, or 15 weeks. In some embodiments, the second phase comprises administration of a combination of a BTK inhibitor and an anti-CD20 therapeutic agent for a period of up to 40 weeks. In some embodiments, the second phase comprises administration of a combination of a BTK inhibitor and an anti-CD20 therapeutic agent for a period of up to 35 weeks. In some embodiments, the second phase comprises administration of a combination of a BTK inhibitor and an anti-CD20 therapeutic agent for a period of up to 30 weeks. In some embodiments, the second phase comprises administration of a combination of a BTK inhibitor and an anti-CD20 therapeutic agent for a period of up to 25 weeks. In some instances, the anti-CD20 therapeutic agent is selected from ofatumumab, rituximab, obinutuzumab, ibritumomab tiuxetan, tositumomab, FBTA05, iodine I 131/tositumomab, obinutuzumab, ocaratuzumab (AME-133v), ocrelizumab, TRU-015, veltuzumab (IMMU-106), or a combination thereof. In some embodiments, the BTK inhibitor is selected from ibrutinib, PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) or LFM-A13. In some embodiments, the BTK inhibitor is ibrutinib.

In some embodiments, the second phase comprises administration of a combination of ibrutinib and an anti-CD20 therapeutic agent for a period of up to 40, 35, 30, 25, 20, or 15 weeks. In some embodiments, the second phase comprises administration of a combination of ibrutinib and an anti-CD20 therapeutic agent for a period of up to 40 weeks. In some embodiments, the second phase comprises administration of a combination of ibrutinib and an anti-CD20 therapeutic agent for a period of up to 35 weeks. In some embodiments, the second phase comprises administration of a combination of ibrutinib and an anti-CD20 therapeutic agent for a period of up to 30 weeks. In some embodiments, the second phase comprises administration of a combination of ibrutinib and an anti-CD20 therapeutic agent for a period of up to 25 weeks. In some instances, the anti-CD20 therapeutic agent is selected from ofatumumab, rituximab, obinutuzumab, ibritumomab tiuxetan, tositumomab, FBTA05, iodine I 131/tositumomab, obinutuzumab, ocaratuzumab (AME-133v), ocrelizumab, TRU-015, veltuzumab (IMMU-106), or a combination thereof. In some instances, the anti-CD20 therapeutic agent is ofatumumab.

In some embodiments, the second phase comprises administration of a combination of ibrutinib and ofatumumab for a period of up to 40, 35, 30, 25, 20, or 15 weeks. In some embodiments, the second phase comprises administration of a combination of ibrutinib and ofatumumab for a period of up to 40 weeks. In some embodiments, the second phase comprises administration of a combination of ibrutinib and ofatumumab for a period of up to 35 weeks. In some embodiments, the second phase comprises administration of a combination of ibrutinib and ofatumumab for a period of up to 30 weeks. In some embodiments, the second phase comprises administration of a combination of ibrutinib and ofatumumab for a period of up to 25 weeks.

In some instances, the combination dosing regimen (i.e. the combined first phase and second phase time) is administered for a period of up to 52 weeks. In some instances, the combination dosing regimen is administered for a period of up to 50, 45, 40, 39, 38, 37, 36, 35, 34, 33, 32, 31, 30, 29, 28, 27, 26, 25, 24, 23, 22, 21, 20, 19, 18, 17, 16, 15, 10, or 5 weeks. In some instances, the combination dosing regimen is administered for a period of up to 37 weeks. In some instances, the combination dosing regimen is administered for a period of up to 29 weeks. In some instances, the combination dosing regimen is administered for a period of up to 27 weeks. In some instances, the combination dosing regimen is administered for a period of up to 25 weeks.

In some embodiments, the amount of a TEC inhibitor that is administered is from 10 mg/day up to, and including, 1000 mg/day. In some embodiments, the amount of a TEC inhibitor that is administered is from about 40 mg/day to 900 mg/day, about 40 mg/day to 840 mg/day, about 80 mg/day to 600 mg/day, about 100 mg/day to 500 mg/day, or about 140 mg/day to 420 mg/day. In some embodiments, the amount of a TEC inhibitor that is administered per day is about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 125 mg, about 130 mg, about 135 mg, about 140 mg, about 180 mg, about 220 mg, about 260 mg, about 300 mg, about 350 mg, about 400 mg, about 420 mg, or about 840 mg.

In some embodiments, the amount of an ITK inhibitor that is administered is from 10 mg/day up to, and including, 1000 mg/day. In some embodiments, the amount of an ITK inhibitor that is administered is from about 40 mg/day to 900 mg/day, about 40 mg/day to 840 mg/day, about 80 mg/day to 600 mg/day, about 100 mg/day to 500 mg/day, or about 140 mg/day to 420 mg/day. In some embodiments, the amount of an ITK inhibitor that is administered per day is about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 125 mg, about 130 mg, about 135 mg, about 140 mg, about 180 mg, about 220 mg, about 260 mg, about 300 mg, about 350 mg, about 400 mg, about 420 mg, or about 840 mg.

In some embodiments, the amount of a BTK inhibitor that is administered is from 10 mg/day up to, and including, 1000 mg/day. In some embodiments, the amount of a BTK inhibitor that is administered is from about 40 mg/day to 900 mg/day, about 40 mg/day to 840 mg/day, about 80 mg/day to 600 mg/day, about 100 mg/day to 500 mg/day, or about 140 mg/day to 420 mg/day. In some embodiments, the amount of a BTK inhibitor that is administered per day is about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 125 mg, about 130 mg, about 135 mg, about 140 mg, about 180 mg, about 220 mg, about 260 mg, about 300 mg, about 350 mg, about 400 mg, about 420 mg, or about 840 mg.

In some embodiments, the amount of ibrutinib that is administered is from 10 mg/day up to, and including, 1000 mg/day. In some embodiments, the amount of Ibrutinib that is administered is from about 40 mg/day to 900 mg/day, about 40 mg/day to 840 mg/day, about 80 mg/day to 600 mg/day, about 100 mg/day to 500 mg/day, or about 140 mg/day to 420 mg/day. In some embodiments, the amount of Ibrutinib that is administered per day is about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 125 mg, about 130 mg, about 135 mg, about 140 mg, about 180 mg, about 220 mg, about 260 mg, about 300 mg, about 350 mg, about 400 mg, about 420 mg, or about 840 mg. In some embodiments, the amount of ibrutinib that is administered is about 40 mg/day. In some embodiments, the amount of ibrutinib that is administered is about 50 mg/day. In some embodiments, the amount of ibrutinib that is administered is about 60 mg/day. In some embodiments, the amount of ibrutinib that is administered is about 70 mg/day. In some embodiments, the amount of ibrutinib that is administered is about 420 mg/day. In some embodiments, the amount of ibrutinib that is administered is about 840 mg/day.

In some embodiments, the TEC inhibitor (e.g. ITK inhibitor or BTK inhibitor) is administered once per day, twice per day, three times per day, once daily, every other day, once a week, twice a week, three times a week, every other week, three times a month, once a month, or intermittently.

In some embodiments, ibrutinib is administered once per day, twice per day, three times per day, once daily, every other day, once a week, twice a week, three times a week, every other week, three times a month, once a month, or intermittently. In some embodiments, ibrutinib is administered once per day. In some embodiments, ibrutinib is administered as a maintenance therapy.

In some embodiments, the TEC inhibitor is administered oral, parenteral (e.g., intravenous, subcutaneous, or intramuscular), buccal, intranasal, rectal or transdermal administration routes. In some embodiments, the TEC inhibitor is administered orally. In some embodiments, the ITK inhibitor is administered orally. In some instances, the BTK inhibitor is administered orally. In some instances, ibrutinib is administered orally.

In some embodiments, the amount of an anti-CD20 therapeutic agent that is administered is from about 50 mg/day to about 5000 mg/day. In some instances, the amount of an anti-CD20 therapeutic agent that is administered is from about 60 mg/day to about 4500 mg/day, from about 80 mg/day to about 4000 mg/day, from about 100 mg/day to about 3500 mg/day, from about 200 mg/day to about 3000 mg/day, or from about 300 mg/day to about 2000 mg/day. In some instances, the amount of an anti-CD20 therapeutic agent that is administered is about 200 mg/day, about 250 mg/day, about 300 mg/day, about 350 mg/day, about 400 mg/day, about 500 mg/day, about 750 mg/day, about 1000 mg/day, about 1500 mg/day, about 2000 mg/day, or about 2500 mg/day. In some instances, the amount of an anti-CD20 therapeutic agent that is administered is about 300 mg/day. In some instances, the amount of an anti-CD20 therapeutic agent that is administered is about 2000 mg/day.

In some instances, the anti-CD20 therapeutic agent is selected from ofatumumab, rituximab, obinutuzumab, ibritumomab tiuxetan, tositumomab, FBTA05, iodine I 131/tositumomab, obinutuzumab, ocaratuzumab (AME-133v), ocrelizumab, TRU-015, or veltuzumab (IMMU-106). In some instances, the anti-CD20 therapeutic agent is ofatumumab.

In some embodiments, the amount of ofatumumab that is administered is from about 50 mg/day to about 5000 mg/day. In some instances, the amount of ofatumumab that is administered is from about 60 mg/day to about 4500 mg/day, from about 80 mg/day to about 4000 mg/day, from about 100 mg/day to about 3500 mg/day, from about 200 mg/day to about 3000 mg/day, or from about 300 mg/day to about 2000 mg/day. In some instances, the amount of ofatumumab that is administered is about 200 mg/day, about 250 mg/day, about 300 mg/day, about 350 mg/day, about 400 mg/day, about 500 mg/day, about 750 mg/day, about 1000 mg/day, about 1500 mg/day, about 2000 mg/day, or about 2500 mg/day. In some instances, the amount of ofatumumab that is administered is about 300 mg/day. In some instances, the amount of ofatumumab that is administered is about 2000 mg/day.

In some embodiments, the anti-CD20 antibody is administered oral, parenteral (e.g., intravenous, subcutaneous, or intramuscular), buccal, intranasal, rectal or transdermal administration routes. In some embodiments, the anti-CD20 antibody is administered intravenously. In some instances, ofatumumab is administered intravenously.

In some embodiments, the anti-CD20 antibody is administered at most 1, 2, 3, 4, 5, 6, 7, 8, 9, 10, 11, 12, 13, 14, 15, 16, 17, 18, 19, 20, 30, 50, or 100 infusions. In some embodiments, the anti-CD20 antibody is administered at most 6, 7, 8, 9, 10, 11, 12, 13, 14, or 15 infusions. In some embodiments, the anti-CD20 antibody is administered at most 12 infusions.

In some embodiments, the anti-CD20 antibody is administered at least 1, 2, 3, 4, 5, 6, 7, 8, 9, 10, 11, 12, 13, 14, 15, 16, 17, 18, 19, 20, 30, 50, or 100 infusions. In some embodiments, the anti-CD20 antibody is administered at least 6, 7, 8, 9, 10, 11, 12, 13, 14, or 15 infusions. In some embodiments, the anti-CD20 antibody is administered at least 12 infusions.

In some embodiments, ofatumumab is administered at most 1, 2, 3, 4, 5, 6, 7, 8, 9, 10, 11, 12, 13, 14, 15, 16, 17, 18, 19, 20, 30, 50, or 100 infusions. In some embodiments, ofatumumab is administered at most 6, 7, 8, 9, 10, 11, 12, 13, 14, or 15 infusions. In some embodiments, ofatumumab is administered at most 12 infusions.

In some embodiments, ofatumumab is administered at least 1, 2, 3, 4, 5, 6, 7, 8, 9, 10, 11, 12, 13, 14, 15, 16, 17, 18, 19, 20, 30, 50, or 100 infusions. In some embodiments, ofatumumab is administered at least 6, 7, 8, 9, 10, 11, 12, 13, 14, or 15 infusions. In some embodiments, ofatumumab is administered at least 12 infusions.

In some embodiments, the compositions disclosed herein are administered for prophylactic, therapeutic, or maintenance treatment. In some embodiments, the compositions disclosed herein are administered for therapeutic applications. In some embodiments, the compositions disclosed herein are administered for therapeutic applications. In some embodiments, the compositions disclosed herein are administered as a maintenance therapy, for example for a patient in remission.

In the case wherein the patient's status does improve, upon the doctor's discretion the administration of the compounds may be given continuously; alternatively, the dose of drug being administered may be temporarily reduced or temporarily suspended for a certain length of time (i.e., a “drug holiday”). The length of the drug holiday can vary between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday may be from 10%400%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained. Patients can, however, require intermittent treatment on a long-term basis upon any recurrence of symptoms.

The amount of a given agent that will correspond to such an amount will vary depending upon factors such as the particular compound, the severity of the disease, the identity (e.g., weight) of the subject or host in need of treatment, but can nevertheless be routinely determined in a manner known in the art according to the particular circumstances surrounding the case, including, e.g., the specific agent being administered, the route of administration, and the subject or host being treated. In general, however, doses employed for adult human treatment will typically be in the range of 0.02-5000 mg per day, or from about 1-1500 mg per day. The desired dose may conveniently be presented in a single dose or as divided doses administered simultaneously (or over a short period of time) or at appropriate intervals, for example as two, three, four or more sub-doses per day.

The pharmaceutical composition described herein may be in unit dosage forms suitable for single administration of precise dosages. In unit dosage form, the formulation is divided into unit doses containing appropriate quantities of one or more compound. The unit dosage may be in the form of a package containing discrete quantities of the formulation. Non-limiting examples are packaged tablets or capsules, and powders in vials or ampoules. Aqueous suspension compositions can be packaged in single-dose non-reclosable containers. Alternatively, multiple-dose reclosable containers can be used, in which case it is typical to include a preservative in the composition. By way of example only, formulations for parenteral injection may be presented in unit dosage form, which include, but are not limited to ampoules, or in multi-dose containers, with an added preservative.

The foregoing ranges are merely suggestive, as the number of variables in regard to an individual treatment regime is large, and considerable excursions from these recommended values are not uncommon. Such dosages may be altered depending on a number of variables, not limited to the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens can be determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it can be expressed as the ratio between LD50 and ED50. Compounds exhibiting high therapeutic indices are preferred. The data obtained from cell culture assays and animal studies can be used in formulating a range of dosage for use in human. The dosage of such compounds lies preferably within a range of circulating concentrations that include the ED50 with minimal toxicity. The dosage may vary within this range depending upon the dosage form employed and the route of administration utilized.

### Btk Inhibitor Compounds and Pharmaceutically Acceptable Salts Thereof

The Btk inhibitor compound described herein (i.e. ibrutinib) is selective for Btk and kinases having a cysteine residue in an amino acid sequence position of the tyrosine kinase that is homologous to the amino acid sequence position of cysteine 481 in Btk. The Btk inhibitor compound can form a covalent bond with Cys 481 of Btk (e.g., via a Michael reaction).

In some embodiments, the Btk inhibitor is a compound of Formula (A) having the structure:

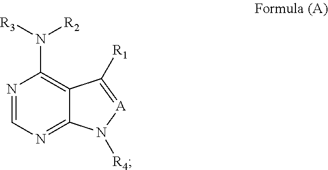

wherein:

A is N;

R₁ is phenyl-O-phenyl or phenyl-S-phenyl;

R₂ and R₃ are independently H;

R₄ is L₃-X-L₄-G, wherein,

L₃ is optional, and when present is a bond, optionally substituted or unsubstituted alkyl, optionally substituted or unsubstituted cycloalkyl, optionally substituted or unsubstituted alkenyl, optionally substituted or unsubstituted alkynyl;

X is optional, and when present is a bond, —O—, —C(═O)—, —S—, —S(═O)—, —S(═O)₂—, —NH—, —NR₉—, —NHC(O)—, —C(O)NH—, —NR₉C(O)—, —C(O)NR₉—, —S(═O)₂NH—, —NHS(═O)₂—, —S(═O)₂NR₉—, —NR₉S(═O)₂—, —OC(O)NH—, —NHC(O)O—, —OC(O)NR₉—, —NR₉C(O)O—, —CH═NO—, —ON═CH—, —NR₁₀C(O)NR₁₀—, heteroaryl-, aryl-, —NR₁₀C(═NR₁₁)NR₁₀—, —NR₁₀C(═NR₁₁)—, —C(═NR₁₁)NR₁₀—, —OC(═NR₁₁)—, or —C(═NR₁₁)O—;

L₄ is optional, and when present is a bond, substituted or unsubstituted alkyl, substituted or unsubstituted cycloalkyl, substituted or unsubstituted alkenyl, substituted or unsubstituted alkynyl, substituted or unsubstituted aryl, substituted or unsubstituted heteroaryl, substituted or unsubstituted heterocycle;

or L₃, X and L₄ taken together form a nitrogen containing heterocyclic ring;

G is

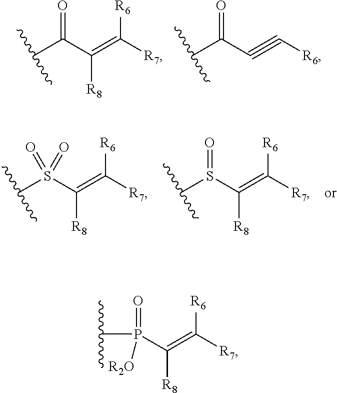

wherein,

R₆, R₇ and R₈ are independently selected from among H, halogen, CN, OH, substituted or unsubstituted alkyl or substituted or unsubstituted heteroalkyl or substituted or unsubstituted cycloalkyl, substituted or unsubstituted heterocycloalkyl, substituted or unsubstituted aryl, substituted or unsubstituted heteroaryl;

each R₉ is independently selected from among H, substituted or unsubstituted lower alkyl, and substituted or unsubstituted lower cycloalkyl;

each R₁₀ is independently H, substituted or unsubstituted lower alkyl, or substituted or unsubstituted lower cycloalkyl; or

two R₁₀ groups can together form a 5-, 6-, 7-, or 8-membered heterocyclic ring; or R₁₀ and R₁₁ can together form a 5-, 6-, 7-, or 8-membered heterocyclic ring; or each R₁₁ is independently selected from H or substituted or unsubstituted alkyl; or a pharmaceutically acceptable salt thereof. In some embodiments, L₃, X and L₄ taken together form a nitrogen containing heterocyclic ring. In some embodiments, the nitrogen containing heterocyclic ring is a piperidine group. In some embodiments, G is

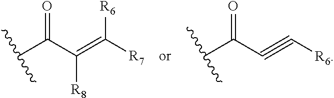

In some embodiments, the compound of Formula (A) is 1-[(3R)-3-[4-amino-3-(4-phenoxyphenyl)pyrazolo[3,4-d]pyrimidin-1-yl]piperidin-1-yl]prop-2-en-1-one.

“Ibrutinib” or “1-((R)-3-(4-amino-3-(4-phenoxyphenyl)-1H-pyrazolo[3,4-d]pyrimidin-1-yl)piperidin-1-yl)prop-2-en-1-one” or “1-{(3R)-3-[4-amino-3-(4-phenoxyphenyl)-1H-pyrazolo[3,4-d]pyrimidin-1-yl]piperidin-1-yl}prop-2-en-1-one” or “2-Propen-1-one, 1-[(3R)-3-[4-amino-3-(4-phenoxyphenyl)-1H-pyrazolo[3,4-d]pyrimidin-1-yl]-1-piperidinyl-” or ibrutinib or any other suitable name refers to the compound with the following structure:

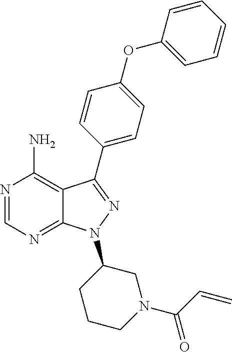 [SMILES: C=CC(=O)N1CCC[C@@H](n2nc(-c3ccc(Oc4ccccc4)cc3)c3c(N)ncnc32)C1]

A wide variety of pharmaceutically acceptable salts is formed from ibrutinib and includes:
acid addition salts formed by reacting ibrutinib with an organic acid, which includes aliphatic mono- and dicarboxylic acids, phenyl-substituted alkanoic acids, hydroxyl alkanoic acids, alkanedioic acids, aromatic acids, aliphatic and aromatic sulfonic acids, amino acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like;
acid addition salts formed by reacting ibrutinib with an inorganic acid, which includes hydrochloric acid, hydrobromic acid, sulfuric acid, nitric acid, phosphoric acid, hydroiodic acid, hydrofluoric acid, phosphorous acid, and the like.

The term “pharmaceutically acceptable salts” in reference to ibrutinib refers to a salt of ibrutinib, which does not cause significant irritation to a mammal to which it is administered and does not substantially abrogate the biological activity and properties of the compound.

It should be understood that a reference to a pharmaceutically acceptable salt includes the solvent addition forms (solvates). Solvates contain either stoichiometric or non-stoichiometric amounts of a solvent, and are formed during the process of product formation or isolation with pharmaceutically acceptable solvents such as water, ethanol, methanol, methyl tert-butyl ether (MTBE), diisopropyl ether (DIPE), ethyl acetate, isopropyl acetate, isopropyl alcohol, methyl isobutyl ketone (MIBK), methyl ethyl ketone (MEK), acetone, nitromethane, tetrahydrofuran (THF), dichloromethane (DCM), dioxane, heptanes, toluene, anisole, acetonitrile, and the like. In one aspect, solvates are formed using, but limited to, Class 3 solvent(s). Categories of solvents are defined in, for example, the International Conference on Harmonization of Technical Requirements for Registration of Pharmaceuticals for Human Use (ICH), “Impurities: Guidelines for Residual Solvents, Q3C(R3), (November 2005). Hydrates are formed when the solvent is water, or alcoholates are formed when the solvent is alcohol. In some embodiments, solvates of ibrutinib, or pharmaceutically acceptable salts thereof, are conveniently prepared or formed during the processes described herein. In some embodiments, solvates of ibrutinib are anhydrous. In some embodiments, ibrutinib, or pharmaceutically acceptable salts thereof, exist in unsolvated form. In some embodiments, ibrutinib, or pharmaceutically acceptable salts thereof, exist in unsolvated form and are anhydrous.

In yet other embodiments, ibrutinib, or a pharmaceutically acceptable salt thereof, is prepared in various forms, including but not limited to, amorphous phase, crystalline forms, milled forms and nano-particulate forms. In some embodiments, ibrutinib, or a pharmaceutically acceptable salt thereof, is amorphous. In some embodiments, ibrutinib, or a pharmaceutically acceptable salt thereof, is amorphous and anhydrous. In some embodiments, ibrutinib, or a pharmaceutically acceptable salt thereof, is crystalline. In some embodiments, ibrutinib, or a pharmaceutically acceptable salt thereof, is crystalline and anhydrous.

In some embodiments, ibrutinib is prepared as outlined in U.S. Pat. No. 7,514,444.

In some embodiments, the Btk inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13.

In some embodiments, the Btk inhibitor is 4-(tert-butyl)-N-(2-methyl-3-(4-methyl-6-44-(morpholine-4-carbonyl)phenyl)amino)-5-oxo-4,5-dihydropyrazin-2-yl)phenyl)benzamide (CGI-1746); 7-benzyl-1-(3-(piperidin-1-yl)propyl)-2-(4-(pyridin-4-yl)phenyl)-1H-imidazo[4,5-g]quinoxalin-6(5H)-one (CTA-056); (R)—N-(3-(6-(4-(1,4-dimethyl-3-oxopiperazin-2-yl)phenylamino)-4-methyl-5-oxo-4,5-dihydropyrazin-2-yl)-2-methylphenyl)-4,5,6,7-tetrahydrobenzo[b]thiophene-2-carboxamide (GDC-0834); 6-cyclopropyl-8-fluoro-2-(2-hydroxymethyl-3-{1-methyl-5-[5-(4-methyl-piperazin-1-yl)-pyridin-2-ylamino]-6-oxo-1,6-dihydro-pyridin-3-yl}-phenyl)-2H-isoquinolin-1-one (RN-486); N-[5-[5-(4-acetylpiperazine-1-carbonyl)-4-methoxy-2-methylphenyl]sulfanyl-1,3-thiazol-2-yl]-4-[(3,3-dimethylbutan-2-ylamino)methyl]benzamide (BMS-509744, HY-11092); or N-(5-45-(4-Acetylpiperazine-1-carbonyl)-4-methoxy-2-methylphenyl)thio)thiazol-2-yl)-4-(((3-methylbutan-2-yl)amino)methyl)benzamide (HY11066); or a pharmaceutically acceptable salt thereof.

In some embodiments, the Btk inhibitor is:

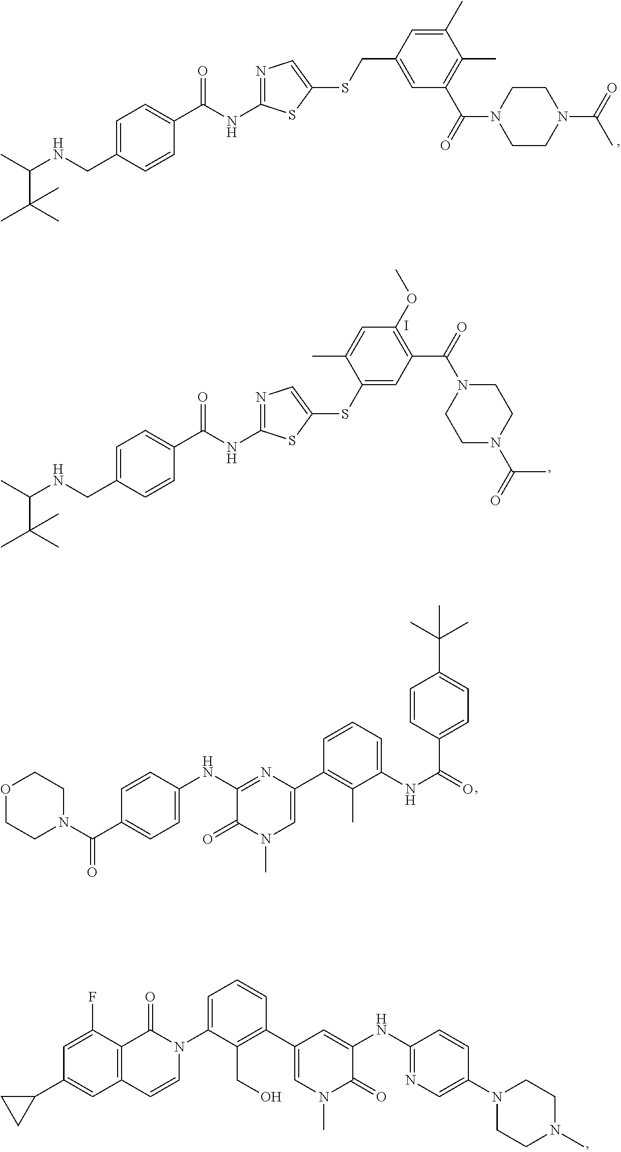

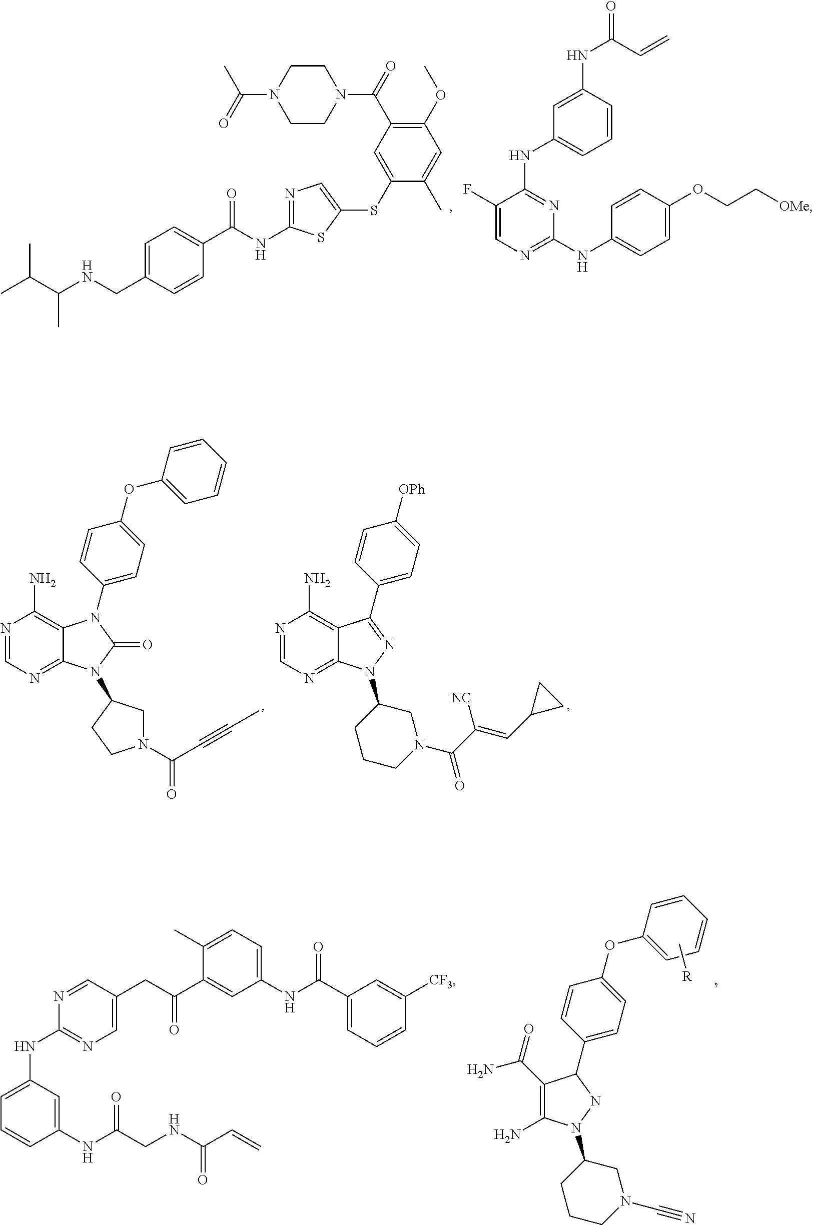

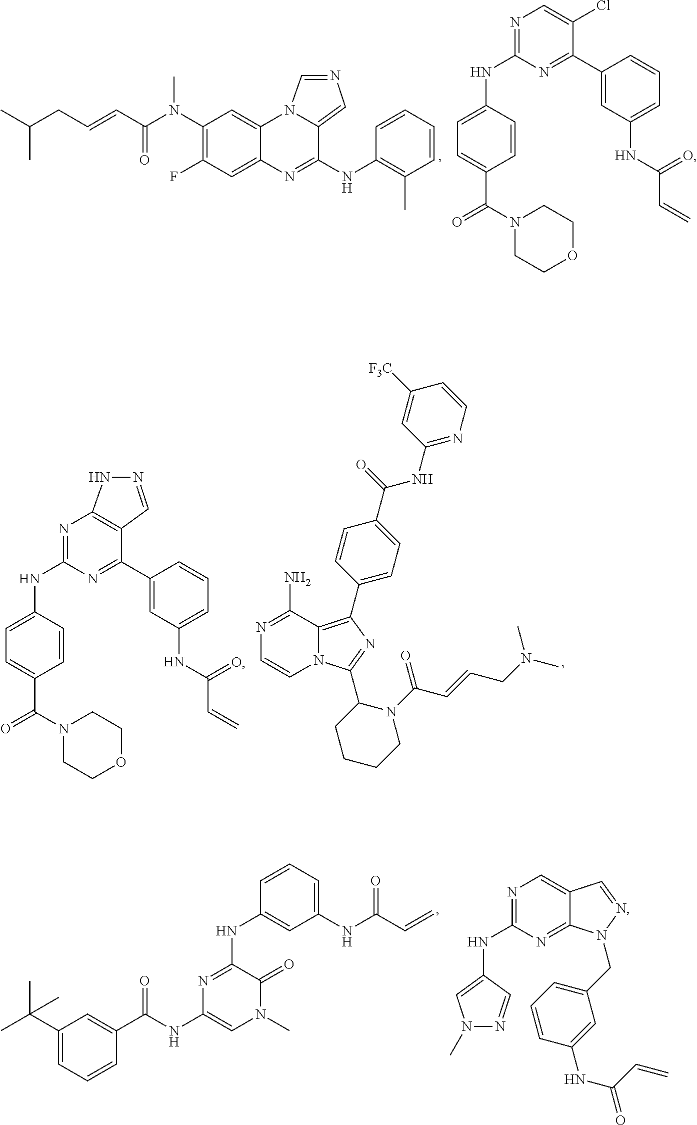

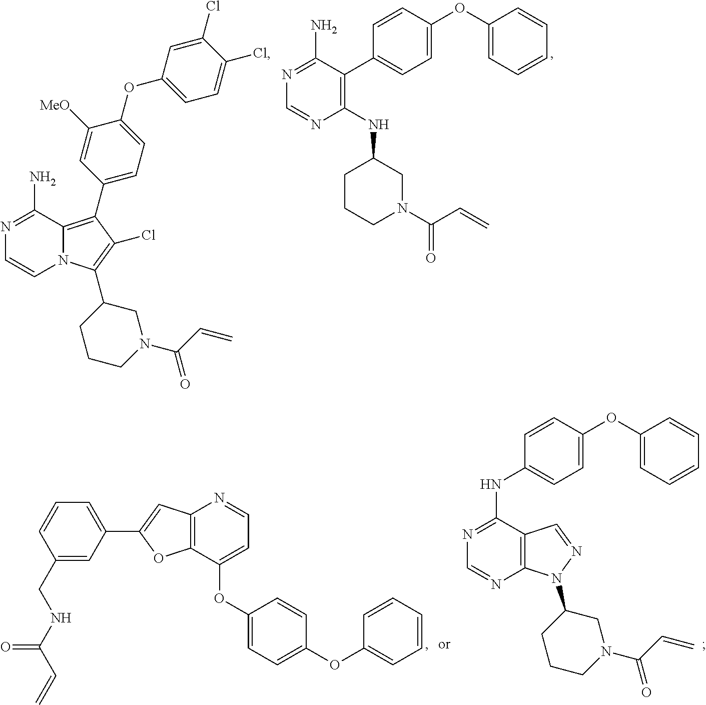

or a pharmaceutically acceptable salt thereof.

### Additional TEC Family Kinase Inhibitors

BTK is a member of the Tyrosine-protein kinase (TEC) family of kinases. In some embodiments, the TEC family comprises BTK, ITK, TEC, RLK and BMX. In some embodiments, a TEC family kinase inhibitor inhibits the kinase activity of BTK, ITK, TEC, RLK and BMX. In some embodiments, a TEC family kinase inhibitor is a BTK inhibitor, which is disclosed elsewhere herein. In some embodiments, a TEC family kinase inhibitor is an ITK inhibitor. In some embodiments, a TEC family kinase inhibitor is a TEC inhibitor. In some embodiments, a TEC family kinase inhibitor is a RLK inhibitor. In some embodiments, a TEC family kinase inhibitor is a BMK inhibitor.

In some embodiments, the ITK inhibitor covalently binds to Cysteine 442 of ITK. In some embodiments, the Itk inhibitor is an Itk inhibitor compound described in WO2002/0500071, which is incorporated by reference in its entirety. In some embodiments, the Itk inhibitor is an Itk inhibitor compound described in WO2005/070420, which is incorporated by reference in its entirety. In some embodiments, the Itk inhibitor is an Itk inhibitor compound described in WO2005/079791, which is incorporated by reference in its entirety. In some embodiments, the Itk inhibitor is an Itk inhibitor compound described in WO2007/076228, which is incorporated by reference in its entirety. In some embodiments, the Itk inhibitor is an Itk inhibitor compound described in WO2007/058832, which is incorporated by reference in its entirety. In some embodiments, the Itk inhibitor is an Itk inhibitor compound described in WO2004/016610, which is incorporated by reference in its entirety. In some embodiments, the Itk inhibitor is an Itk inhibitor compound described in WO2004/016611, which is incorporated by reference in its entirety. In some embodiments, the Itk inhibitor is an Itk inhibitor compound described in WO2004/016600, which is incorporated by reference in its entirety. In some embodiments, the Itk inhibitor is an Itk inhibitor compound described in WO2004/016615, which is incorporated by reference in its entirety. In some embodiments, the Itk inhibitor is an Itk inhibitor compound described in WO2005/026175, which is incorporated by reference in its entirety. In some embodiments, the Itk inhibitor is an Itk inhibitor compound described in WO2006/065946, which is incorporated by reference in its entirety. In some embodiments, the Itk inhibitor is an Itk inhibitor compound described in WO2007/027594, which is incorporated by reference in its entirety. In some embodiments, the Itk inhibitor is an Itk inhibitor compound described in WO2007/017455, which is incorporated by reference in its entirety. In some embodiments, the Itk inhibitor is an Itk inhibitor compound described in WO2008/025820, which is incorporated by reference in its entirety. In some embodiments, the Itk inhibitor is an Itk inhibitor compound described in WO2008/025821, which is incorporated by reference in its entirety. In some embodiments, the Itk inhibitor is an Itk inhibitor compound described in WO2008/025822, which is incorporated by reference in its entirety. In some embodiments, the Itk inhibitor is an Itk inhibitor compound described in WO2011/017219, which is incorporated by reference in its entirety. In some embodiments, the Itk inhibitor is an Itk inhibitor compound described in WO2011/090760, which is incorporated by reference in its entirety. In some embodiments, the Itk inhibitor is an Itk inhibitor compound described in WO2009/158571, which is incorporated by reference in its entirety. In some embodiments, the Itk inhibitor is an Itk inhibitor compound described in WO2009/051822, which is incorporated by reference in its entirety. In some embodiments, the Itk inhibitor is an Itk inhibitor compound described in US 20110281850, which is incorporated by reference in its entirety. In some embodiments, the Itk inhibitor is an Itk inhibitor compound described in WO2014/082085, which is incorporated by reference in its entirety. In some embodiments, the Itk inhibitor is an Itk inhibitor compound described in WO2014/093383, which is incorporated by reference in its entirety. In some embodiments, the Itk inhibitor is an Itk inhibitor compound described in U.S. Pat. No. 8,759,358, which is incorporated by reference in its entirety. In some embodiments, the Itk inhibitor is an Itk inhibitor compound described in WO2014/105958, which is incorporated by reference in its entirety. In some embodiments, the Itk inhibitor is an Itk inhibitor compound described in US2014/0256704, which is incorporated by reference in its entirety. In some embodiments, the Itk inhibitor is an Itk inhibitor compound described in US20140315909, which is incorporated by reference in its entirety. In some embodiments, the Itk inhibitor is an Itk inhibitor compound described in US20140303161, which is incorporated by reference in its entirety. In some embodiments, the Itk inhibitor is an Itk inhibitor compound described in WO2014/145403, which is incorporated by reference in its entirety.

In some embodiments, the Itk inhibitor has a structure selected from:

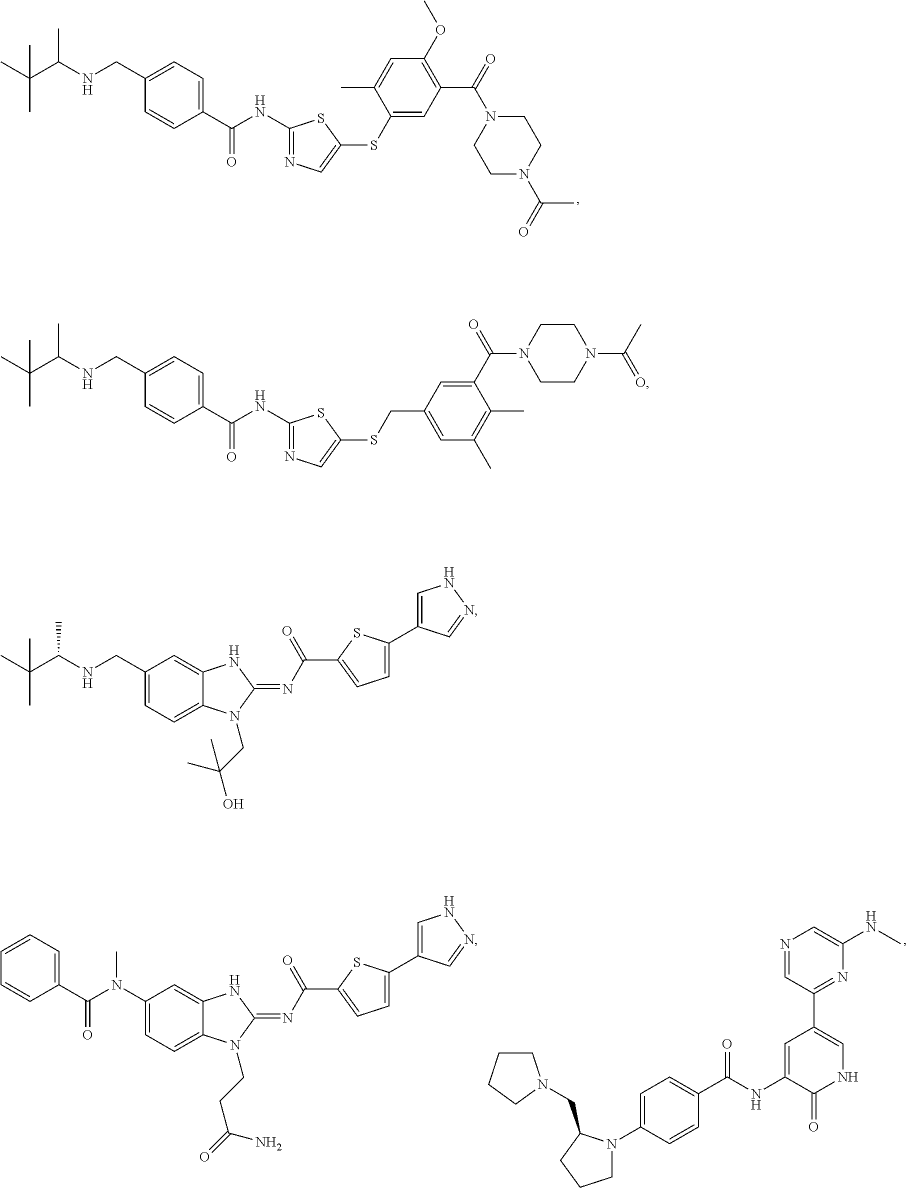

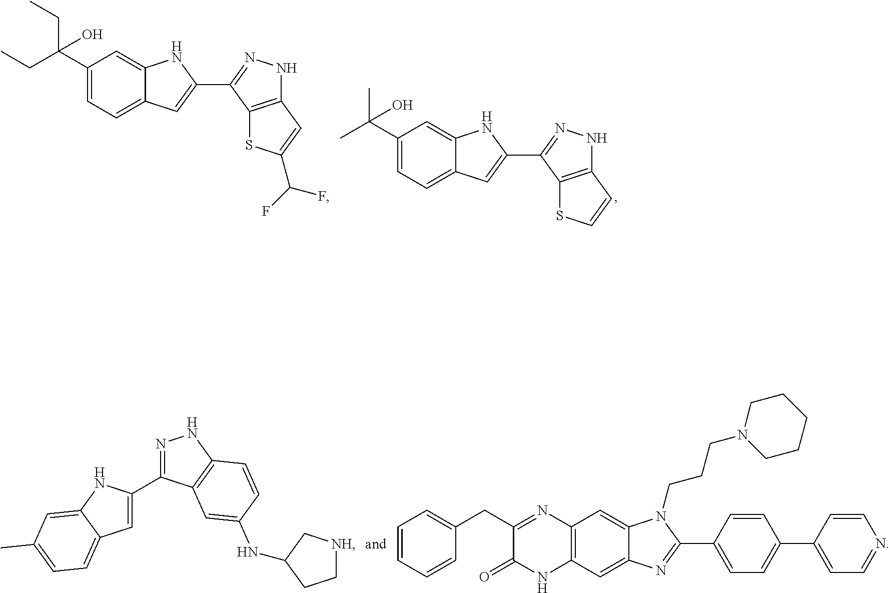

### Hematologic Malignancies

Disclosed herein are methods and combination dosing regimen for administering a combination of a TEC inhibitor and an anti-CD20 therapeutic agent for the treatment of a hematologic malignancy. In some embodiments, the hematologic malignancy is a leukemia, a lymphoma, a myeloma, a non-Hodgkin's lymphoma, a Hodgkin's lymphoma, a T-cell malignancy, or a B-cell malignancy.

In some embodiments, the hematologic malignancy is a T-cell malignancy. In some embodiments, the T-cell malignancy is peripheral T-cell lymphoma not otherwise specified (PTCL-NOS), anaplastic large cell lymphoma, angioimmunoblastic lymphoma, cutaneous T-cell lymphoma, adult T-cell leukemia/lymphoma (ATLL), blastic NK-cell lymphoma, enteropathy-type T-cell lymphoma, hematosplenic gamma-delta T-cell lymphoma, lymphoblastic lymphoma, nasal NK/T-cell lymphomas, or treatment-related T-cell lymphomas.

In some embodiments, the hematologic malignancy is a B-cell proliferative disorder. In some embodiments, the cancer is chronic lymphocytic leukemia (CLL), small lymphocytic lymphoma (SLL), high risk CLL, a non-CLL/SLL lymphoma, or prolymphocytic leukemia (PLL). In some embodiments, the cancer is follicular lymphoma (FL), diffuse large B-cell lymphoma (DLBCL), mantle cell lymphoma (MCL), Waldenström's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, DLBCL is further divided into subtypes: activated B-cell diffuse large B-cell lymphoma (ABC-DLBCL), germinal center diffuse large B-cell lymphoma (GCB DLBCL), and Double-Hit (DH) DLBCL. In some embodiments, ABC-DLBCL is characterized by a CD79B mutation. In some embodiments, ABC-DLBCL is characterized by a CD79A mutation. In some embodiments, the ABC-DLBCL is characterized by a mutation in MyD88, A20, or a combination thereof. In some embodiments, the cancer is acute or chronic myelogenous (or myeloid) leukemia, myelodysplastic syndrome, or acute lymphoblastic leukemia.

In some embodiments, the cancer is diffuse large B-cell lymphoma (DLBCL). In some embodiments, the cancer is activated B-cell diffuse large B-cell lymphoma (ABC-DLBCL). In some embodiments, the cancer is follicular lymphoma (FL). In some embodiments, the cancer is multiple myeloma. In some embodiments, the cancer is chronic lymphocytic leukemia (CLL). In some embodiments, the cancer is small lymphocytic lymphoma (SLL). In some embodiments, the cancer is non-CLL/SLL lymphoma. In some embodiments, the cancer is high risk CLL or high risk SLL. In some embodiments, the cancer is PLL. In some embodiments, the cancer is MCL. In some embodiments, the cancer is Waldenström's macroglobulinemia.

In some embodiments, a cancer is a treatment-naive cancer. In some instances, a treatment-naive cancer is a cancer that has not been treated by a therapy, such as for example by a 1EC inhibitor, an anti-CD20 therapeutic agent, and/or by an additional therapeutic agent disclosed elsewhere herein. In some embodiments, a treatment-naive cancer is a hematologic cancer.

In some embodiments, the treatment-naive hematologic cancer is a leukemia, a lymphoma, a myeloma, a non-Hodgkin's lymphoma, a Hodgkin's lymphoma, a T-cell malignancy, or a B-cell malignancy. In some embodiments, the treatment-naive hematologic cancer is a B-cell malignancy. In some embodiments, the B-cell malignancy is chronic lymphocytic leukemia (CLL), small lymphocytic lymphoma (SLL), high risk CLL, non-CLL/SLL lymphoma, prolymphocytic leukemia (PLL), follicular lymphoma (FL), diffuse large B-cell lymphoma (DLBCL), mantle cell lymphoma (MCL), Waldenström's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, the treatment-naive hematologic cancer is CLL. In some embodiments, the treatment-naive hematologic cancer is SLL. In some embodiments, the treatment-naive hematologic cancer is DLBCL. In some embodiments, the treatment-naive hematologic cancer is mantle cell lymphoma. In some embodiments, the treatment-naive hematologic cancer is FL. In some embodiments, the treatment-naive hematologic cancer is Waldenstrom's macroglobulinemia. In some embodiments, the treatment-naive hematologic cancer is multiple myeloma. In some embodiments, the treatment-naive hematologic cancer is Burkitt's lymphoma. In some embodiments, the treatment-naive hematologic cancer is PLL.

In some embodiments, described herein methods and combination dosing regimen for administering a combination of a 1EC inhibitor (e.g. ITK inhibitor or a BTK inhibitor) and an anti-CD20 therapeutic agent for the treatment of a hematologic malignancy selected from chronic lymphocytic leukemia (CLL), small lymphocytic lymphoma (SLL), high risk CLL, non-CLL/SLL lymphoma, prolymphocytic leukemia (PLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

In some embodiments, described herein methods and combination dosing regimen for administering a combination of a TEC inhibitor (e.g. an ITK inhibitor or a BTK inhibitor) and an anti-CD20 therapeutic agent for the treatment of CLL.

In some embodiments, described herein methods and combination dosing regimen for administering a combination of a TEC inhibitor (e.g. an ITK inhibitor or a BTK inhibitor) and an anti-CD20 therapeutic agent for the treatment of SLL.

In some embodiments, described herein methods and combination dosing regimen for administering a combination of a TEC inhibitor (e.g. an ITK inhibitor or a BTK inhibitor) and an anti-CD20 therapeutic agent for the treatment of PLL.

In some embodiments, described herein methods and combination dosing regimen for administering a combination of a TEC inhibitor (e.g. an ITK inhibitor or a BTK inhibitor) an anti-CD20 therapeutic agent for the treatment of DLBCL.

In some embodiments, described herein methods and combination dosing regimen for administering a combination of a TEC inhibitor (e.g. an ITK inhibitor or a BTK inhibitor) and an anti-CD20 therapeutic agent for the treatment of MCL.

In some embodiments, described herein methods and combination dosing regimen for administering a combination of a TEC inhibitor (e.g. an ITK inhibitor or a BTK inhibitor) and an anti-CD20 therapeutic agent for the treatment of Waldenström's macroglobulinemia.

In some embodiments, described herein methods and combination dosing regimen for administering a combination of a BTK inhibitor and an anti-CD20 therapeutic agent for the treatment of a hematologic malignancy selected from chronic lymphocytic leukemia (CLL), small lymphocytic lymphoma (SLL), high risk CLL, non-CLL/SLL lymphoma, prolymphocytic leukemia (PLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

In some embodiments, described herein methods and combination dosing regimen for administering a combination of a BTK inhibitor and an anti-CD20 therapeutic agent for the treatment of CLL.

In some embodiments, described herein methods and combination dosing regimen for administering a combination of a BTK inhibitor and an anti-CD20 therapeutic agent for the treatment of SLL.

In some embodiments, described herein methods and combination dosing regimen for administering a combination of a BTK inhibitor and an anti-CD20 therapeutic agent for the treatment of PLL.

In some embodiments, described herein methods and combination dosing regimen for administering a combination of a BTK inhibitor and an anti-CD20 therapeutic agent for the treatment of DLBCL.

In some embodiments, described herein methods and combination dosing regimen for administering a combination of a BTK inhibitor and an anti-CD20 therapeutic agent for the treatment of MCL.

In some embodiments, described herein methods and combination dosing regimen for administering a combination of a BTK inhibitor and an anti-CD20 therapeutic agent for the treatment of Waldenström's macroglobulinemia.

In some embodiments, described herein methods and combination dosing regimen for administering a combination of ibrutinib and an anti-CD20 therapeutic agent for the treatment of a hematologic malignancy selected from chronic lymphocytic leukemia (CLL), small lymphocytic lymphoma (SLL), high risk CLL, non-CLL/SLL lymphoma, prolymphocytic leukemia (PLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

In some embodiments, described herein methods and combination dosing regimen for administering a combination of ibrutinib and an anti-CD20 therapeutic agent for the treatment of CLL.

In some embodiments, described herein methods and combination dosing regimen for administering a combination of ibrutinib and an anti-CD20 therapeutic agent for the treatment of SLL.

In some embodiments, described herein methods and combination dosing regimen for administering a combination of ibrutinib and an anti-CD20 therapeutic agent for the treatment of PLL.

In some embodiments, described herein methods and combination dosing regimen for administering a combination of ibrutinib an anti-CD20 therapeutic agent for the treatment of DLBCL.

In some embodiments, described herein methods and combination dosing regimen for administering a combination of ibrutinib and an anti-CD20 therapeutic agent for the treatment of MCL.

In some embodiments, described herein methods and combination dosing regimen for administering a combination of ibrutinib and an anti-CD20 therapeutic agent for the treatment of Waldenström's macroglobulinemia.

In some embodiments, described herein methods and combination dosing regimen for administering a combination of a TEC inhibitor (e.g. an ITK inhibitor or a BTK inhibitor such as ibrutinib) and an anti-CD20 therapeutic agent for the treatment of a treatment-naive hematologic malignancy selected from chronic lymphocytic leukemia (CLL), small lymphocytic lymphoma (SLL), high risk CLL, non-CLL/SLL lymphoma, prolymphocytic leukemia (PLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

### Relapsed or Refractory Hematologic Malignancy

In some embodiments, the hematologic cancer is a relapsed or refractory hematologic cancer. In some embodiments, the relapsed or refractory hematologic cancer is a leukemia, a lymphoma, a myeloma, a non-Hodgkin's lymphoma, a Hodgkin's lymphoma, T-cell malignancy, or a B-cell malignancy.

In some embodiments, the relapsed or refractory hematologic cancer is a T-cell malignancy. In some embodiments, the relapsed or refractory T-cell malignancy is peripheral T-cell lymphoma not otherwise specified (PTCL-NOS), anaplastic large cell lymphoma, angioimmunoblastic lymphoma, cutaneous T-cell lymphoma, adult T-cell leukemia/lymphoma (ATLL), blastic NK-cell lymphoma, enteropathy-type T-cell lymphoma, hematosplenic gamma-delta T-cell lymphoma, lymphoblastic lymphoma, nasal NK/T-cell lymphomas, or treatment-related T-cell lymphomas.

In some embodiments, the relapsed or refractory hematologic cancer is a B-cell proliferative disorder. In some embodiments, the relapsed or refractory cancer is chronic lymphocytic leukemia (CLL), small lymphocytic lymphoma (SLL), high risk CLL, a non-CLL/SLL lymphoma, or prolymphocytic leukemia (PLL). In some embodiments, the cancer is follicular lymphoma, diffuse large B-cell lymphoma (DLBCL), mantle cell lymphoma (MCL), Waldenström's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, the relapsed or refractory DLBCL is further divided into subtypes: activated B-cell diffuse large B-cell lymphoma (ABC-DLBCL), germinal center diffuse large B-cell lymphoma (GCB DLBCL), and Double-Hit (DH) DLBCL. In some embodiments, ABC-DLBCL is characterized by a CD79B mutation. In some embodiments, ABC-DLBCL is characterized by a CD79A mutation. In some embodiments, the ABC-DLBCL is characterized by a mutation in MyD88, A20, or a combination thereof. In some embodiments, the cancer is acute or chronic myelogenous (or myeloid) leukemia, myelodysplastic syndrome, or acute lymphoblastic leukemia.

In some embodiments, the cancer is relapsed or refractory diffuse large B-cell lymphoma (DLBCL). In some embodiments, the cancer is relapsed or refractory activated B-cell diffuse large B-cell lymphoma (ABC-DLBCL). In some embodiments, the cancer is relapsed or refractory follicular lymphoma (FL). In some embodiments, the cancer is relapsed or refractory multiple myeloma. In some embodiments, the cancer is relapsed or refractory chronic lymphocytic leukemia (CLL). In some embodiments, the cancer is relapsed or refractory small lymphocytic lymphoma (SLL). In some embodiments, the cancer is relapsed or refractory non-CLL/SLL lymphoma. In some embodiments, the cancer is relapsed or refractory high risk CLL or high risk SLL. In some embodiments, the cancer is relapsed or refractory PLL. In some embodiments, the cancer is relapsed or refractory MCL. In some embodiments, the cancer is relapsed or refractory Waldenström's macroglobulinemia.

In some embodiments, described herein methods and combination dosing regimen for administering a combination of a TEC inhibitor (e.g. ITK inhibitor or a BTK inhibitor) and an anti-CD20 therapeutic agent for the treatment of a relapsed or refractory hematologic malignancy selected from chronic lymphocytic leukemia (CLL), small lymphocytic lymphoma (SLL), high risk CLL, non-CLL/SLL lymphoma, prolymphocytic leukemia (PLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

In some embodiments, described herein methods and combination dosing regimen for administering a combination of a TEC inhibitor (e.g. an ITK inhibitor or a BTK inhibitor) and an anti-CD20 therapeutic agent for the treatment of relapsed or refractory CLL.

In some embodiments, described herein methods and combination dosing regimen for administering a combination of a TEC inhibitor (e.g. an ITK inhibitor or a BTK inhibitor) and an anti-CD20 therapeutic agent for the treatment of relapsed or refractory SLL.

In some embodiments, described herein methods and combination dosing regimen for administering a combination of a TEC inhibitor (e.g. an ITK inhibitor or a BTK inhibitor) and an anti-CD20 therapeutic agent for the treatment of relapsed or refractory PLL.

In some embodiments, described herein methods and combination dosing regimen for administering a combination of a TEC inhibitor (e.g. an ITK inhibitor or a BTK inhibitor) an anti-CD20 therapeutic agent for the treatment of relapsed or refractory DLBCL.

In some embodiments, described herein methods and combination dosing regimen for administering a combination of a TEC inhibitor (e.g. an ITK inhibitor or a BTK inhibitor) and an anti-CD20 therapeutic agent for the treatment of relapsed or refractory MCL.

In some embodiments, described herein methods and combination dosing regimen for administering a combination of a TEC inhibitor (e.g. an ITK inhibitor or a BTK inhibitor) and an anti-CD20 therapeutic agent for the treatment of relapsed or refractory Waldenström's macroglobulinemia.

In some embodiments, described herein methods and combination dosing regimen for administering a combination of a BTK inhibitor and an anti-CD20 therapeutic agent for the treatment of a relapsed or refractory hematologic malignancy selected from chronic lymphocytic leukemia (CLL), small lymphocytic lymphoma (SLL), high risk CLL, non-CLL/SLL lymphoma, prolymphocytic leukemia (PLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

In some embodiments, described herein methods and combination dosing regimen for administering a combination of a BTK inhibitor and an anti-CD20 therapeutic agent for the treatment of relapsed or refractory CLL.

In some embodiments, described herein methods and combination dosing regimen for administering a combination of a BTK inhibitor and an anti-CD20 therapeutic agent for the treatment of relapsed or refractory SLL.

In some embodiments, described herein methods and combination dosing regimen for administering a combination of a BTK inhibitor and an anti-CD20 therapeutic agent for the treatment of relapsed or refractory PLL.

In some embodiments, described herein methods and combination dosing regimen for administering a combination of a BTK inhibitor and an anti-CD20 therapeutic agent for the treatment of relapsed or refractory DLBCL.

In some embodiments, described herein methods and combination dosing regimen for administering a combination of a BTK inhibitor and an anti-CD20 therapeutic agent for the treatment of relapsed or refractory MCL.

In some embodiments, described herein methods and combination dosing regimen for administering a combination of a BTK inhibitor and an anti-CD20 therapeutic agent for the treatment of relapsed or refractory Waldenström's macroglobulinemia.

In some embodiments, described herein methods and combination dosing regimen for administering a combination of ibrutinib and an anti-CD20 therapeutic agent for the treatment of a relapsed or refractory hematologic malignancy selected from chronic lymphocytic leukemia (CLL), small lymphocytic lymphoma (SLL), high risk CLL, non-CLL/SLL lymphoma, prolymphocytic leukemia (PLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

In some embodiments, described herein methods and combination dosing regimen for administering a combination of ibrutinib and an anti-CD20 therapeutic agent for the treatment of relapsed or refractory CLL.

In some embodiments, described herein methods and combination dosing regimen for administering a combination of ibrutinib and an anti-CD20 therapeutic agent for the treatment of relapsed or refractory SLL.

In some embodiments, described herein methods and combination dosing regimen for administering a combination of ibrutinib and an anti-CD20 therapeutic agent for the treatment of relapsed or refractory PLL.

In some embodiments, described herein methods and combination dosing regimen for administering a combination of ibrutinib an anti-CD20 therapeutic agent for the treatment of relapsed or refractory DLBCL.

In some embodiments, described herein methods and combination dosing regimen for administering a combination of ibrutinib and an anti-CD20 therapeutic agent for the treatment of relapsed or refractory MCL.

In some embodiments, described herein methods and combination dosing regimen for administering a combination of ibrutinib and an anti-CD20 therapeutic agent for the treatment of relapsed or refractory Waldenström's macroglobulinemia.

In some embodiments, the relapsed or refractory hematologic cancer is a relapsed or refractory ibrutinib-resistant hematologic cancer. In some embodiments, described herein methods and combination dosing regimen for administering a combination of ibrutinib and an anti-CD20 therapeutic agent for the treatment of a relapsed or refractory ibrutinib-resistant hematologic malignancy selected from chronic lymphocytic leukemia (CLL), small lymphocytic lymphoma (SLL), high risk CLL, non-CLL/SLL lymphoma, prolymphocytic leukemia (PLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

### Metastasized Hematologic Malignancy

In some embodiments, the hematologic cancer is a metastasized hematologic cancer. In some embodiments, the metastasized hematologic cancer is a leukemia, a lymphoma, a myeloma, a non-Hodgkin's lymphoma, a Hodgkin's lymphoma, a T-cell malignancy, or a B-cell malignancy.

In some embodiments, the metastasized hematologic cancer is a T-cell malignancy. In some embodiments, the T-cell malignancy is peripheral T-cell lymphoma not otherwise specified (PTCL-NOS), anaplastic large cell lymphoma, angioimmunoblastic lymphoma, cutaneous T-cell lymphoma, adult T-cell leukemia/lymphoma (ATLL), blastic NK-cell lymphoma, enteropathy-type T-cell lymphoma, hematosplenic gamma-delta T-cell lymphoma, lymphoblastic lymphoma, nasal NK/T-cell lymphomas, or treatment-related T-cell lymphomas.

In some embodiments, the metastasized hematologic cancer is a B-cell proliferative disorder. In some embodiments, the metastasized hematologic cancer is chronic lymphocytic leukemia (CLL), small lymphocytic lymphoma (SLL), high risk CLL, a non-CLL/SLL lymphoma, or prolymphocytic leukemia (PLL). In some embodiments, the metastasized hematologic cancer is follicular lymphoma (FL), diffuse large B-cell lymphoma (DLBCL), mantle cell lymphoma (MCL), Waldenström's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, DLBCL is further divided into subtypes: activated B-cell diffuse large B-cell lymphoma (ABC-DLBCL), germinal center diffuse large B-cell lymphoma (GCB DLBCL), and Double-Hit (DH) DLBCL. In some embodiments, ABC-DLBCL is characterized by a CD79B mutation. In some embodiments, ABC-DLBCL is characterized by a CD79A mutation. In some embodiments, the ABC-DLBCL is characterized by a mutation in MyD88, A20, or a combination thereof. In some embodiments, the cancer is acute or chronic myelogenous (or myeloid) leukemia, myelodysplastic syndrome, or acute lymphoblastic leukemia.

In some embodiments, the metastasized hematologic cancer is diffuse large B-cell lymphoma (DLBCL). In some embodiments, the metastasized hematologic cancer is activated B-cell diffuse large B-cell lymphoma (ABC-DLBCL). In some embodiments, the metastasized hematologic cancer is follicular lymphoma (FL). In some embodiments, the metastasized hematologic cancer is multiple myeloma. In some embodiments, the metastasized hematologic cancer is chronic lymphocytic leukemia (CLL). In some embodiments, the metastasized hematologic cancer is small lymphocytic lymphoma (SLL). In some embodiments, the metastasized hematologic cancer is non-CLL/SLL lymphoma. In some embodiments, the metastasized hematologic cancer is high risk CLL or high risk SLL. In some embodiments, the metastasized hematologic cancer is PLL. In some embodiments, the metastasized hematologic cancer is MCL. In some embodiments, the metastasized hematologic cancer is Waldenström's macroglobulinemia.

In some embodiments, described herein methods and combination dosing regimen for administering a combination of a rEC inhibitor (e.g. ITK inhibitor or a BTK inhibitor) and an anti-CD20 therapeutic agent for the treatment of a metastasized hematologic malignancy selected from chronic lymphocytic leukemia (CLL), small lymphocytic lymphoma (SLL), high risk CLL, non-CLL/SLL lymphoma, prolymphocytic leukemia (PLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

In some embodiments, described herein methods and combination dosing regimen for administering a combination of a rEC inhibitor (e.g. an ITK inhibitor or a BTK inhibitor) and an anti-CD20 therapeutic agent for the treatment of metastasized CLL.

In some embodiments, described herein methods and combination dosing regimen for administering a combination of a TEC inhibitor (e.g. an ITK inhibitor or a BTK inhibitor) and an anti-CD20 therapeutic agent for the treatment of metastasized SLL.

In some embodiments, described herein methods and combination dosing regimen for administering a combination of a TEC inhibitor (e.g. an ITK inhibitor or a BTK inhibitor) and an anti-CD20 therapeutic agent for the treatment of metastasized PLL.

In some embodiments, described herein methods and combination dosing regimen for administering a combination of a TEC inhibitor (e.g. an ITK inhibitor or a BTK inhibitor) an anti-CD20 therapeutic agent for the treatment of metastasized DLBCL.

In some embodiments, described herein methods and combination dosing regimen for administering a combination of a TEC inhibitor (e.g. an ITK inhibitor or a BTK inhibitor) and an anti-CD20 therapeutic agent for the treatment of metastasized MCL.

In some embodiments, described herein methods and combination dosing regimen for administering a combination of a TEC inhibitor (e.g. an ITK inhibitor or a BTK inhibitor) and an anti-CD20 therapeutic agent for the treatment of metastasized Waldenström's macroglobulinemia.

In some embodiments, described herein methods and combination dosing regimen for administering a combination of a BTK inhibitor and an anti-CD20 therapeutic agent for the treatment of a metastasized hematologic malignancy selected from chronic lymphocytic leukemia (CLL), small lymphocytic lymphoma (SLL), high risk CLL, non-CLL/SLL lymphoma, prolymphocytic leukemia (PLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

In some embodiments, described herein methods and combination dosing regimen for administering a combination of a BTK inhibitor and an anti-CD20 therapeutic agent for the treatment of metastasized CLL.

In some embodiments, described herein methods and combination dosing regimen for administering a combination of a BTK inhibitor and an anti-CD20 therapeutic agent for the treatment of metastasized SLL.

In some embodiments, described herein methods and combination dosing regimen for administering a combination of a BTK inhibitor and an anti-CD20 therapeutic agent for the treatment of metastasized PLL.

In some embodiments, described herein methods and combination dosing regimen for administering a combination of a BTK inhibitor and an anti-CD20 therapeutic agent for the treatment of metastasized DLBCL.

In some embodiments, described herein methods and combination dosing regimen for administering a combination of a BTK inhibitor and an anti-CD20 therapeutic agent for the treatment of metastasized MCL.

In some embodiments, described herein methods and combination dosing regimen for administering a combination of a BTK inhibitor and an anti-CD20 therapeutic agent for the treatment of metastasized Waldenström's macroglobulinemia.

In some embodiments, described herein methods and combination dosing regimen for administering a combination of ibrutinib and an anti-CD20 therapeutic agent for the treatment of a metastasized hematologic malignancy selected from chronic lymphocytic leukemia (CLL), small lymphocytic lymphoma (SLL), high risk CLL, non-CLL/SLL lymphoma, prolymphocytic leukemia (PLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

In some embodiments, described herein methods and combination dosing regimen for administering a combination of ibrutinib and an anti-CD20 therapeutic agent for the treatment of metastasized CLL.

In some embodiments, described herein methods and combination dosing regimen for administering a combination of ibrutinib and an anti-CD20 therapeutic agent for the treatment of metastasized SLL.

In some embodiments, described herein methods and combination dosing regimen for administering a combination of ibrutinib and an anti-CD20 therapeutic agent for the treatment of metastasized PLL.

In some embodiments, described herein methods and combination dosing regimen for administering a combination of ibrutinib an anti-CD20 therapeutic agent for the treatment of metastasized DLBCL.

In some embodiments, described herein methods and combination dosing regimen for administering a combination of ibrutinib and an anti-CD20 therapeutic agent for the treatment of metastasized MCL.

In some embodiments, described herein methods and combination dosing regimen for administering a combination of ibrutinib and an anti-CD20 therapeutic agent for the treatment of metastasized Waldenström's macroglobulinemia.

### Additional Therapeutic Agents

Disclosed herein include methods and combination dosing regimen of administering a combination of a TEC inhibitor (e.g. an ITK inhibitor or a BTK inhibitor), an anti-CD20 therapeutic agent, and an additional therapeutic agent. In some embodiments, the additional therapeutic agent is a chemotherapeutic agent, a steroid, analgesic, an immunotherapeutic agent, a targeted therapy, or a combination thereof. In some embodiments, the additional therapeutic agent is a B cell receptor pathway inhibitor. In some embodiments, the B cell receptor pathway inhibitor is a CD79A inhibitor, a CD79B inhibitor, a CD19 inhibitor, a Lyn inhibitor, a Syk inhibitor, a PI3K inhibitor, a Blk inhibitor, a PLCγ inhibitor, a PKCβ inhibitor, or a combination thereof. In some embodiments, the additional therapeutic agent is an antibody, B cell receptor signaling inhibitor, a PI3K inhibitor, an IAP inhibitor, an mTOR inhibitor, a radioimmunotherapeutic, a DNA damaging agent, a proteosome inhibitor, a histone deacetylase inhibitor, a protein kinase inhibitor, a hedgehog inhibitor, an Hsp90 inhibitor, a telomerase inhibitor, a Jak1/2 inhibitor, a protease inhibitor, a PKC inhibitor, a PARP inhibitor, or a combination thereof.

In some embodiments, the additional therapeutic agent comprises an analgesic such as acetaminophen.

In some embodiments, the additional therapeutic agent comprises an agent selected from: an inhibitor of LYN, SYK, JAK, PI3K, PLCγ, MAPK, MEK or NFκB.

In some embodiments, the additional therapeutic agent comprises an agent selected from: bendamustine, bortezomib, lenalidomide, idelalisib (GS-1101), vorinostat, everolimus, panobinostat, temsirolimus, romidepsin, vorinostat, fludarabine, cyclophosphamide, mitoxantrone, pentostatine, prednisone, etopside, procarbazine, and thalidomide.

In some embodiments the additional therapeutic agent is bendamustine. In some embodiments, bortezomib is administered in combination with rituximab.

In some embodiments, the additional therapeutic agent is bortezomib. In some embodiments, bendamustine is administered in combination with rituximab.

In some embodiments, the additional therapeutic agent is lenalidomide. In some embodiments, lenalidomide is administered in combination with rituximab.

In some embodiments, the additional therapeutic agent is a multi-agent therapeutic regimen. In some embodiments the additional therapeutic agent comprises the HyperCVAD regimen (cyclophosphamide, vincristine, doxorubicin, dexamethasone alternating with methotrexate and cytarabine). In some embodiments, the HyperCVAD regimen is administered in combination with rituximab.

In some embodiments the additional therapeutic agent comprises the R-CHOP regiment (rituximab, cyclophosphamide, doxorubicin, vincristine, and prednisone).

In some embodiments the additional therapeutic agent comprises the FCR regimen (FCR (fludarabine, cyclophosphamide, rituximab).

In some embodiments the additional therapeutic agent comprises the FCMR regimen (fludarabine, cyclophosphamide, mitoxantrone, rituximab).

In some embodiments the additional therapeutic agent comprises the FMR regimen (fludarabine, mitoxantrone, rituximab).

In some embodiments the additional therapeutic agent comprises the PCR regimen (pentostatin, cyclophosphamide, rituximab).

In some embodiments the additional therapeutic agent comprises the PEPC regimen (prednisone, etoposide, procarbazine, cyclophosphamide).

In some embodiments the additional therapeutic agent comprises radioimmunotherapy with ⁹⁰Y-ibritumomab tiuxetan or ¹³¹I-tositumomab.

In some embodiments, the additional therapeutic agent is an autologous stem cell transplant.

In some embodiments, the additional therapeutic agent is selected from: nitrogen mustards such as for example, bendamustine, chlorambucil, chlormethine, cyclophosphamide, ifosfamide, melphalan, prednimustine, trofosfamide; alkyl sulfonates like busulfan, mannosulfan, treosulfan; ethylene imines like carboquone, thiotepa, triaziquone; nitrosoureas like carmustine, fotemustine, lomustine, nimustine, ranimustine, semustine, streptozocin; epoxides such as for example, etoglucid; other alkylating agents such as for example dacarbazine, mitobronitol, pipobroman, temozolomide; folic acid analogues such as for example methotrexate, permetrexed, pralatrexate, raltitrexed; purine analogs such as for example cladribine, clofarabine, fludarabine, mercaptopurine, nelarabine, tioguanine; pyrimidine analogs such as for example azacitidine, capecitabine, carmofur, cytarabine, decitabine, fluorouracil, gemcitabine, tegafur; vinca alkaloids such as for example vinblastine, vincristine, vindesine, vinflunine, vinorelbine; podophyllotoxin derivatives such as for example etoposide, teniposide; colchicine derivatives such as for example demecolcine; taxanes such as for example docetaxel, paclitaxel, paclitaxel poliglumex; other plant alkaloids and natural products such as for example trabectedin; actinomycines such as for example dactinomycin; antracyclines such as for example aclarubicin, daunorubicin, doxorubicin, epirubicin, idarubicin, mitoxantrone, pirarubicin, valrubicin, zorubincin; other cytotoxic antibiotics such as for example bleomycin, ixabepilone, mitomycin, plicamycin; platinum compounds such as for example carboplatin, cisplatin, oxaliplatin, satraplatin; methylhydrazines such as for example procarbazine; sensitizers such as for example aminolevulinic acid, efaproxiral, methyl aminolevulinate, porfimer sodium, temoporfin; protein kinase inhibitors such as for example dasatinib, erlotinib, everolimus, gefitinib, imatinib, lapatinib, nilotinib, pazonanib, sorafenib, sunitinib, temsirolimus; other antineoplastic agents such as for example alitretinoin, altretamine, amzacrine, anagrelide, arsenic trioxide, asparaginase, bexarotene, bortezomib, celecoxib, denileukin diftitox, estramustine, hydroxycarbamide, irinotecan, lonidamine, masoprocol, miltefosein, mitoguazone, mitotane, oblimersen, pegaspargase, pentostatin, romidepsin, sitimagene ceradenovec, tiazofurine, topotecan, tretinoin, vorinostat; estrogens such as for example diethylstilbenol, ethinylestradiol, fosfestrol, polyestradiol phosphate; progestogens such as for example gestonorone, medroxyprogesterone, megestrol; gonadotropin releasing hormone analogs such as for example buserelin, goserelin, leuprorelin, triptorelin; anti-estrogens such as for example fulvestrant, tamoxifen, toremifene; anti-androgens such as for example bicalutamide, flutamide, nilutamide; enzyme inhibitors, aminoglutethimide, anastrozole, exemestane, formestane, letrozole, vorozole; other hormone antagonists such as for example abarelix, degarelix; immunostimulants such as for example histamine dihydrochloride, mifamurtide, pidotimod, plerixafor, roquinimex, thymopentin; immunosuppressants such as for example everolimus, gusperimus, leflunomide, mycophenolic acid, sirolimus; calcineurin inhibitors such as for example ciclosporin, tacrolimus; other immunosuppressants such as for example azathioprine, lenalidomide, methotrexate, thalidomide; and radiopharmaceuticals such as for example, iobenguane.

In some embodiments, the additional therapeutic agent is selected from: interferons, interleukins, tumor necrosis factors, growth factors, or the like.

In some embodiments, the additional therapeutic agent is selected from: ancestim, filgrastim, lenograstim, molgramostim, pegfilgrastim, sargramostim; interferons such as for example interferon alfa natural, interferon alfa-2a, interferon alfa-2b, interferon alfacon-1, interferon alfa-n1, interferon beta natural, interferon beta-1a, interferon beta-1b, interferon gamma, peginterferon alfa-2a, peginterferon alfa-2b; interleukins such as for example aldesleukin, oprelvekin; other immunostimulants such as for example BCG vaccine, glatiramer acetate, histamine dihydrochloride, immunocyanin, lentinan, melanoma vaccine, mifamurtide, pegademase, pidotimod, plerixafor, poly I:C, poly ICLC, roquinimex, tasonermin, thymopentin; immunosuppressants such as for example abatacept, abetimus, alefacept, antilymphocyte immunoglobulin (horse), antithymocyte immunoglobulin (rabbit), eculizumab, efalizumab, everolimus, gusperimus, leflunomide, muromab-CD3, mycophenolic acid, natalizumab, sirolimus; TNF alpha Inhibitors such as for example adalimumab, afelimomab, certolizumab pegol, etanercept, golimumab, infliximab; Interleukin Inhibitors such as for example anakinra, basiliximab, canakinumab, daclizumab, mepolizumab, rilonacept, tocilizumab, ustekinumab; calcineurin inhibitors such as for example ciclosporin, tacrolimus; other immunosuppressants such as for example azathioprine, lenalidomide, methotrexate, thalidomide.

In some embodiments, the additional therapeutic agent is selected from: adalimumab, alemtuzumab, basiliximab, bevacizumab, cetuximab, certolizumab pegol, daclizumab, eculizumab, efalizumab, gemtuzumab, ibritumomab tiuxetan, infliximab, muromonab-CD3, natalizumab, panitumumab, ranibizumab, tositumomab, trastuzumab, or the like, or a combination thereof.

In some embodiments, the additional therapeutic agent is selected from: monoclonal antibodies such as for example alemtuzumab, bevacizumab, catumaxomab, cetuximab, edrecolomab, gemtuzumab, panitumumab, trastuzumab; immunosuppressants, eculizumab, efalizumab, muromab-CD3, natalizumab; TNF alpha inhibitors such as for example adalimumab, afelimomab, certolizumab pegol, golimumab, infliximab; interleukin inhibitors, basiliximab, canakinumab, daclizumab, mepolizumab, tocilizumab, ustekinumab; radiopharmaceuticals, ibritumomab tiuxetan, tositumomab; others monoclonal antibodies such as for example abagovomab, adecatumumab, alemtuzumab, anti-CD30 monoclonal antibody Xmab2513, anti-MET monoclonal antibody MetMab, apolizumab, apomab, arcitumomab, basiliximab, bispecific antibody 2B1, blinatumomab, brentuximab vedotin, capromab pendetide, cixutumumab, claudiximab, conatumumab, dacetuzumab, denosumab, eculizumab, epratuzumab, epratuzumab, ertumaxomab, etaracizumab, figitumumab, fresolimumab, galiximab, ganitumab, gemtuzumab ozogamicin, glembatumumab, ibritumomab, inotuzumab ozogamicin, ipilimumab, lexatumumab, lintuzumab, lintuzumab, lucatumumab, mapatumumab, matuzumab, milatuzumab, monoclonal antibody CC49, necitumumab, nimotuzumab, oregovomab, pertuzumab, ramacurimab, ranibizumab, siplizumab, sonepcizumab, tanezumab, tositumomab, trastuzumab, tremelimumab, tucotuzumab celmoleukin, veltuzumab, visilizumab, volociximab, zalutumumab.

In some embodiments, the additional therapeutic agent is selected from: agents that affect the tumor micro-environment such as cellular signaling network (e.g. phosphatidylinositol 3-kinase (PI3K) signaling pathway, signaling from the B-cell receptor and the IgE receptor). In some embodiments, the additional therapeutic agent is a PI3K signaling inhibitor or a syc kinase inhibitor. In one embodiment, the syk inhibitor is R788. In another embodiment is a PKCγ inhibitor such as by way of example only, enzastaurin.

Examples of agents that affect the tumor micro-environment include PI3K signaling inhibitor, syc kinase inhibitor, protein kinase inhibitors such as for example dasatinib, erlotinib, everolimus, gefitinib, imatinib, lapatinib, nilotinib, pazonanib, sorafenib, sunitinib, temsirolimus; other angiogenesis inhibitors such as for example GT-111, JI-101, R1530; other kinase inhibitors such as for example AC220, AC480, ACE-041, AMG 900, AP24534, Arry-614, AT7519, AT9283, AV-951, axitinib, AZD1152, AZD7762, AZD8055, AZD8931, bafetinib, BAY 73-4506, BGJ398, BGT226, BI 811283, BI6727, BIBF 1120, BIBW 2992, BMS-690154, BMS-777607, BMS-863233, BSK-461364, CAL-101, CEP-11981, CYC116, DCC-2036, dinaciclib, dovitinib lactate, E7050, EMD 1214063, ENMD-2076, fostamatinib disodium, GSK2256098, GSK690693, INCB18424, INNO-406, JNJ-26483327, JX-594, KX2-391, linifanib, LY2603618, MGCD265, MK-0457, MK1496, MLN8054, MLN8237, MP470, NMS-1116354, NMS-1286937, ON 01919.Na, OSI-027, OSI-930, Btk inhibitor, PF-00562271, PF-02341066, PF-03814735, PF-04217903, PF-04554878, PF-04691502, PF-3758309, PHA-739358, PLC3397, progenipoietin, R547, R763, ramucirumab, regorafenib, RO5185426, SAR103168, SCH 727965, SGI-1176, SGX523, SNS-314, TAK-593, TAK-901, TKI258, TLN-232, TTP607, XL147, XL228, XL281RO5126766, XL418, XL765.

In some embodiments, the additional therapeutic agent is selected from: inhibitors of mitogen-activated protein kinase signaling, e.g., U0126, PD98059, PD184352, PD0325901, ARRY-142886, SB239063, SP600125, BAY 43-9006, wortmannin, or LY294002; Syk inhibitors; mTOR inhibitors; and antibodies (e.g., rituxan).

In some embodiments, the additional therapeutic agent is selected from: adriamycin, dactinomycin, bleomycin, vinblastine, cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; iimofosine; interleukin Il (including recombinant interleukin II, or rlL2), interferon alfa-2a; interferon alfa-2b; interferon alfa-n1; interferon alfa-n3; interferon beta-1 a; interferon gamma-1 b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

In some embodiments, the additional therapeutic agent is selected from: 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-such as for example growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

In some embodiments, the additional therapeutic agent is selected from: alkylating agents, antimetabolites, natural products, or hormones, e.g., nitrogen mustards (e.g., mechloroethamine, cyclophosphamide, chlorambucil, etc.), alkyl sulfonates (e.g., busulfan), nitrosoureas (e.g., carmustine, lomusitne, ete.), or triazenes (decarbazine, etc.). Examples of antimetabolites include but are not limited to folic acid analog (e.g., methotrexate), or pyrimidine analogs (e.g., Cytarabine), purine analogs (e.g., mercaptopurine, thioguanine, pentostatin).

In some embodiments, the additional therapeutic agent is selected from: nitrogen mustards (e.g., mechloroethamine, cyclophosphamide, chlorambucil, meiphalan, etc.), ethylenimine and methylmelamines (e.g., hexamethlymelamine, thiotepa), alkyl sulfonates (e.g., busulfan), nitrosoureas (e.g., carmustine, lomusitne, semustine, streptozocin, etc.), or triazenes (decarbazine, ete.). Examples of antimetabolites include, but are not limited to folic acid analog (e.g., methotrexate), or pyrimidine analogs (e.g., fluorouracil, floxouridine, Cytarabine), purine analogs (e.g., mercaptopurine, thioguanine, pentostatin.

In some embodiments, the additional therapeutic agent is selected from: agents which act by arresting cells in the G2-M phases due to stabilized microtubules, e.g., erbulozole (also known as R-55104), dolastatin 10 (also known as DLS-10 and NSC-376128), mivobulin isethionate (also known as CI-980), vincristine, NSC-639829, discodermolide (also known as NVP-XX-A-296), ABT-751 (Abbott, also known as E-7010), altorhyrtins (such as Altorhyrtin A and Altorhyrtin C), spongistatins (such as Spongistatin 1, Spongistatin 2, Spongistatin 3, Spongistatin 4, Spongistatin 5, Spongistatin 6, Spongistatin 7, Spongistatin 8, and Spongistatin 9), cemadotin hydrochloride (also known as LU-103793 and NSC-D-669356), epothilones (such as Epothilone A, Epothilone B, Epothilone C (also known as desoxyepothilone A or dEpoA), Epothilone D (also referred to as KOS-862, dEpoB, and desoxyepothilone B), Epothilone E, Epothilone F, Epothilone B N-oxide, Epothilone A N-oxide, 16-aza-epothilone B, 21-aminoepothilone B (also known as BMS-310705), 21-hydroxyepothilone D (also known as desoxyepothilone F and dEpoF), 26-fluoroepothilone), auristatin PE (also known as NSC-654663), soblidotin (also known as TZT-1027), LS-4559-P (Pharmacia, also known as LS-4577), LS-4578 (Pharmacia, also known as LS-477-P), LS-4477 (Pharmacia), LS-4559 (Pharmacia), RPR-112378 (Aventis), vincristine sulfate, DZ-3358 (Daiichi), FR-182877 (Fujisawa, also known as WS-9885B), GS-164 (Takeda), GS-198 (Takeda), KAR-2 (Hungarian Academy of Sciences), BSF-223651 (BASF, also known as ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), cryptophycin 52 (also known as LY-355703), AC-7739 (Ajinomoto, also known as AVE-8063A and CS-39.HCl), AC-7700 (Ajinomoto, also known as AVE-8062, AVE-8062A, CS-39-L-Ser.HCl, and RPR-258062A), vitilevuamide, tubulysin A, canadensol, centaureidin (also known as NSC-106969), T-138067 (Tularik, also known as T-67, TL-138067 and TI-138067), COBRA-1 (Parker Hughes Institute, also known as DDE-261 and WHI-261), H10 (Kansas State University), H16 (Kansas State University), Oncocidin A1 (also known as BTO-956 and DIME), DDE-313 (Parker Hughes Institute), Fijianolide B, Laulimalide, SPA-2 (Parker Hughes Institute), SPA-1 (Parker Hughes Institute, also known as SPIKET-P), 3-IAABU (Cytoskeleton/Mt. Sinai School of Medicine, also known as MF-569), narcosine (also known as NSC-5366), nascapine, D-24851 (Asta Medica), A-105972 (Abbott), hemiasterlin, 3-BAABU (Cytoskeleton/Mt. Sinai School of Medicine, also known as MF-191), TMPN (Arizona State University), vanadocene acetylacetonate, T-138026 (Tularik), monsatrol, lnanocine (also known as NSC-698666), 3-1AABE (Cytoskeleton/Mt. Sinai School of Medicine), A-204197 (Abbott), T-607 (Tuiarik, also known as T-900607), RPR-115781 (Aventis), eleutherobins (such as Desmethyleleutherobin, Desaetyleleutherobin, lsoeleutherobin A, and Z-Eleutherobin), caribaeoside, caribaeolin, halichondrin B, D-64131 (Asta Medica), D-68144 (Asta Medica), diazonamide A, A-293620 (Abbott), NPI-2350 (Nereus), taccalonolide A, TUB-245 (Aventis), A-259754 (Abbott), diozostatin, (−)-phenylahistin (also known as NSCL-96F037), D-68838 (Asta Medica), D-68836 (Asta Medica), Myoseverin B, D-43411 (Zentaris, also known as D-81862), A-289099 (Abbott), A-318315 (Abbott), HTI-286 (also known as SPA-110, trifluoroacetate salt) (Wyeth), D-82317 (Zentaris), D-82318 (Zentaris), SC-12983 (NCI), resverastatin phosphate sodium, BPR-OY-007 (National Health Research Institutes), and SSR-250411 (Sanofi).

### Pharmaceutical Compositions/Formulations

Disclosed herein, in certain embodiments, are compositions for treating a B cell proliferative disorder in an individual in need thereof comprising a TEC inhibitor (e.g., an ITK inhibitor, a BTK inhibitor, e.g. a covalent BTK inhibitor,) and/or an anti-CD20 therapeutic agent. Disclosed herein, in certain embodiments, are compositions for treating a B cell proliferative disorder in an individual in need thereof comprising a covalent Btk inhibitor (e.g., an irreversible covalent BTK inhibitor, e.g., ibrutinib) and/or an anti-CD20 therapeutic agent. In some embodiments, the B cell proliferative disorder is refractory to the covalent BTK inhibitor (e.g., an irreversible covalent BTK inhibitor, e.g., ibrutinib). In some embodiments, the B cell proliferative disorder is relapsed. In some embodiments, the B cell proliferative disorder is mantle cell lymphoma.

In some embodiments, the covalent BTK inhibitor is a compound of Formula (A). In some embodiments, the covalent Btk inhibitor is (R)-1-(3-(4-amino-3-(4-phenoxyphenyl)-1H-pyrazolo[3,4-d]pyrimidin-1-yl)piperidin-1-yl)prop-2-en-1-one (i.e. PCI-32765/ibrutinib).

Pharmaceutical compositions of covalent Btk inhibitors (e.g., an irreversible covalent Btk inhibitor, e.g., ibrutinib) and/or anti-CD20 therapeutic agents are formulated in a conventional manner using one or more physiologically acceptable carriers including excipients and auxiliaries which facilitate processing of the active compounds into preparations which can be used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

A pharmaceutical composition, as used herein, refers to a mixture of a covalent Btk inhibitor (e.g., an irreversible covalent Btk inhibitor, e.g., ibrutinib) and/or an anti-CD20 therapeutic agent with other chemical components, such as carriers, stabilizers, diluents, dispersing agents, suspending agents, thickening agents, and/or excipients.

Pharmaceutical compositions are optionally manufactured in a conventional manner, such as, by way of example only, by means of conventional mixing, dissolving, granulating, dragee-making, levigating, emulsifying, encapsulating, entrapping or compression processes.

In certain embodiments, compositions may also include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In other embodiments, compositions may also include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound described herein and a co-agent, are both administered to a patient simultaneously in the form of a single entity or dosage. The term “non-fixed combination” means that the active ingredients, e.g. a compound described herein and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The pharmaceutical formulations described herein are administered by any suitable administration route, including but not limited to, oral, parenteral (e.g., intravenous, subcutaneous, intramuscular), intranasal, buccal, topical, rectal, or transdermal administration routes.

The pharmaceutical compositions described herein are formulated into any suitable dosage form, including but not limited to, aqueous oral dispersions, liquids, gels, syrups, elixirs, slurries, suspensions and the like, for oral ingestion by an individual to be treated, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations. In some embodiments, the compositions are formulated into capsules. In some embodiments, the compositions are formulated into solutions (for example, for IV administration).

The pharmaceutical solid dosage forms described herein optionally include a compound described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof.

In still other aspects, using standard coating procedures, such as those described in Remington's Pharmaceutical Sciences, 20th Edition (2000), a film coating is provided around the compositions. In some embodiments, the compositions are formulated into particles (for example for administration by capsule) and some or all of the particles are coated. In some embodiments, the compositions are formulated into particles (for example for administration by capsule) and some or all of the particles are microencapsulated. In some embodiments, the compositions are formulated into particles (for example for administration by capsule) and some or all of the particles are not microencapsulated and are uncoated.

In certain embodiments, compositions provided herein may also include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

“Antifoaming agents” reduce foaming during processing which can result in coagulation of aqueous dispersions, bubbles in the finished film, or generally impair processing. Exemplary anti-foaming agents include silicon emulsions or sorbitan sesquoleate.

“Antioxidants” include, for example, butylated hydroxytoluene (BHT), sodium ascorbate, ascorbic acid, sodium metabisulfite and tocopherol. In certain embodiments, antioxidants enhance chemical stability where required.

Formulations described herein may benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (1) pentosan polysulfate and other heparinoids, (m) divalent cations such as magnesium and zinc; or (n) combinations thereof.

“Binders” impart cohesive qualities and include, e.g., alginic acid and salts thereof; cellulose derivatives such as carboxymethylcellulose, methylcellulose (e.g., Methocel), hydroxypropylmethylcellulose, hydroxyethylcellulose, hydroxypropylcellulose (e.g., Klucel), ethylcellulose (e.g., Ethocel®), and microcrystalline cellulose (e.g., Avicel®); microcrystalline dextrose; amylose; magnesium aluminum silicate; polysaccharide acids; bentonites; gelatin; polyvinylpyrrolidone/vinyl acetate copolymer; crospovidone; povidone; starch; pregelatinized starch; tragacanth, dextrin, a sugar, such as sucrose (e.g., Dipac®), glucose, dextrose, molasses, mannitol, sorbitol, xylitol (e.g., Xylitab®), and lactose; a natural or synthetic gum such as acacia, tragacanth, ghatti gum, mucilage of isapol husks, polyvinylpyrrolidone (e.g., Polyvidone® CL, Kollidon® CL, Polyplasdone® XL-10), larch arabogalactan, Veegum®, polyethylene glycol, waxes, sodium alginate, and the like.

A “carrier” or “carrier materials” include any commonly used excipients in pharmaceutics and should be selected on the basis of compatibility with compounds disclosed herein, such as, compounds of ibrutinib and an anticancer agent, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. “Pharmaceutically compatible carrier materials” may include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

“Dispersing agents,” and/or “viscosity modulating agents” include materials that control the diffusion and homogeneity of a drug through liquid media or a granulation method or blend method. In some embodiments, these agents also facilitate the effectiveness of a coating or eroding matrix. Exemplary diffusion facilitators/dispersing agents include, e.g., hydrophilic polymers, electrolytes, Tween® 60 or 80, PEG, polyvinylpyrrolidone (PVP; commercially known as Plasdone®), and the carbohydrate-based dispersing agents such as, for example, hydroxypropyl celluloses (e.g., HPC, HPC-SL, and HPC-L), hydroxypropyl methylcelluloses (e.g., HPMC K100, HPMC K4M, HPMC K15M, and HPMC K100M), carboxymethylcellulose sodium, methylcellulose, hydroxyethylcellulose, hydroxypropylcellulose, hydroxypropylmethylcellulose phthalate, hydroxypropylmethylcellulose acetate stearate (HPMCAS), noncrystalline cellulose, magnesium aluminum silicate, triethanolamine, polyvinyl alcohol (PVA), vinyl pyrrolidone/vinyl acetate copolymer (S630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)), polyvinylpyrrolidone K12, polyvinylpyrrolidone K17, polyvinylpyrrolidone K25, or polyvinylpyrrolidone K30, polyvinylpyrrolidone/vinyl acetate copolymer (S-630), polyethylene glycol, e.g., the polyethylene glycol can have a molecular weight of about 300 to about 6000, or about 3350 to about 4000, or about 7000 to about 5400, sodium carboxymethylcellulose, methylcellulose, polysorbate-80, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, polysorbate-80, sodium alginate, polyethoxylated sorbitan monolaurate, polyethoxylated sorbitan monolaurate, povidone, carbomers, polyvinyl alcohol (PVA), alginates, chitosans and combinations thereof. Plasticizers such as cellulose or triethyl cellulose can also be used as dispersing agents. Dispersing agents particularly useful in liposomal dispersions and self-emulsifying dispersions are dimyristoyl phosphatidyl choline, natural phosphatidyl choline from eggs, natural phosphatidyl glycerol from eggs, cholesterol and isopropyl myristate.

Combinations of one or more erosion facilitator with one or more diffusion facilitator can also be used in the present compositions.

The term “diluent” refers to chemical compounds that are used to dilute the compound of interest prior to delivery. Diluents can also be used to stabilize compounds because they can provide a more stable environment. Salts dissolved in buffered solutions (which also can provide pH control or maintenance) are utilized as diluents in the art, including, but not limited to a phosphate buffered saline solution. In certain embodiments, diluents increase bulk of the composition to facilitate compression or create sufficient bulk for homogenous blend for capsule filling. Such compounds include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

The term “disintegrate” includes both the dissolution and dispersion of the dosage form when contacted with gastrointestinal fluid. “Disintegration agents or disintegrants” facilitate the breakup or disintegration of a substance. Examples of disintegration agents include a starch, e.g., a natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel®PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

“Drug absorption” or “absorption” typically refers to the process of movement of drug from site of administration of a drug across a barrier into a blood vessel or the site of action, e.g., a drug moving from the gastrointestinal tract into the portal vein or lymphatic system.

An “enteric coating” is a substance that remains substantially intact in the stomach but dissolves and releases the drug in the small intestine or colon. Generally, the enteric coating comprises a polymeric material that prevents release in the low pH environment of the stomach but that ionizes at a higher pH, typically a pH of 6 to 7, and thus dissolves sufficiently in the small intestine or colon to release the active agent therein.

“Erosion facilitators” include materials that control the erosion of a particular material in gastrointestinal fluid. Erosion facilitators are generally known to those of ordinary skill in the art. Exemplary erosion facilitators include, e.g., hydrophilic polymers, electrolytes, proteins, peptides, and amino acids.

“Filling agents” include compounds such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

“Flavoring agents” and/or “sweeteners” useful in the formulations described herein, include, e.g., acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sylitol, sucralose, sorbitol, Swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof.

“Lubricants” and “glidants” are compounds that prevent, reduce or inhibit adhesion or friction of materials. Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and the like.

A “measurable serum concentration” or “measurable plasma concentration” describes the blood serum or blood plasma concentration, typically measured in mg, μg, or ng of therapeutic agent per mL, dL, or L of blood serum, absorbed into the bloodstream after administration. As used herein, measurable plasma concentrations are typically measured in ng/ml or μg/ml.

“Pharmacodynamics” refers to the factors which determine the biologic response observed relative to the concentration of drug at a site of action.

“Pharmacokinetics” refers to the factors which determine the attainment and maintenance of the appropriate concentration of drug at a site of action.

“Plasticizers” are compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. In some embodiments, plasticizers can also function as dispersing agents or wetting agents.

“Solubilizers” include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

“Stabilizers” include compounds such as any antioxidation agents, buffers, acids, preservatives and the like.

“Steady state,” as used herein, is when the amount of drug administered is equal to the amount of drug eliminated within one dosing interval resulting in a plateau or constant plasma drug exposure.

“Suspending agents” include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

“Surfactants” include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Some other surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. In some embodiments, surfactants may be included to enhance physical stability or for other purposes.

“Viscosity enhancing agents” include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

“Wetting agents” include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

### Dosage Forms

The compositions described herein can be formulated for administration to a subject via any conventional means including, but not limited to, oral, parenteral (e.g., intravenous, subcutaneous, or intramuscular), buccal, intranasal, rectal or transdermal administration routes. In some embodiments, the composition is formulated for administration in a combined dosage form. In some embodiments, the composition is formulated for administration in a separate dosage forms. As used herein, the term “subject” is used to mean an animal, preferably a mammal, including a human or non-human. The terms “individual(s)”, “subject(s)” and “patient(s)” are used interchangeably herein, and mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

Moreover, the pharmaceutical compositions described herein, which include ibrutinib and/or an anticancer agent can be formulated into any suitable dosage form, including but not limited to, aqueous oral dispersions, liquids, gels, syrups, elixirs, slurries, suspensions and the like, for oral ingestion by a patient to be treated, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

Pharmaceutical preparations for oral use can be obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents may be added, such as the cross-linked croscarmellose sodium, polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

In some embodiments, the solid dosage forms disclosed herein may be in the form of a tablet, (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder) a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In other embodiments, the pharmaceutical formulation is in the form of a powder. In still other embodiments, the pharmaceutical formulation is in the form of a tablet, including but not limited to, a fast-melt tablet. Additionally, pharmaceutical formulations described herein may be administered as a single capsule or in multiple capsule dosage form. In some embodiments, the pharmaceutical formulation is administered in two, or three, or four, capsules or tablets.

In some embodiments, solid dosage forms, e.g., tablets, effervescent tablets, and capsules, are prepared by mixing particles of ibrutinib and/or an anticancer agent, with one or more pharmaceutical excipients to form a bulk blend composition. When referring to these bulk blend compositions as homogeneous, it is meant that the particles of ibrutinib and/or an anticancer agent, are dispersed evenly throughout the composition so that the composition may be readily subdivided into equally effective unit dosage forms, such as tablets, pills, and capsules. The individual unit dosages may also include film coatings, which disintegrate upon oral ingestion or upon contact with diluent. These formulations can be manufactured by conventional pharmacological techniques.

Conventional pharmacological techniques include, e.g., one or a combination of methods: (1) dry mixing, (2) direct compression, (3) milling, (4) dry or non-aqueous granulation, (5) wet granulation, or (6) fusion. See, e.g., Lachman et al., The Theory and Practice of Industrial Pharmacy (1986). Other methods include, e.g., spray drying, pan coating, melt granulation, granulation, fluidized bed spray drying or coating (e.g., wurster coating), tangential coating, top spraying, tableting, extruding and the like.

The pharmaceutical solid dosage forms described herein can include a compound described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof. In still other aspects, using standard coating procedures, such as those described in Remington's Pharmaceutical Sciences, 20th Edition (2000), a film coating is provided around the formulation of ibrutinib and/or an anticancer agent. In another embodiment, some or all of the particles of ibrutinib and/or an anticancer agent, are not microencapsulated and are uncoated.

Suitable carriers for use in the solid dosage forms described herein include, but are not limited to, acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol and the like.

Suitable filling agents for use in the solid dosage forms described herein include, but are not limited to, lactose, calcium carbonate, calcium phosphate, dibasic calcium phosphate, calcium sulfate, microcrystalline cellulose, cellulose powder, dextrose, dextrates, dextran, starches, pregelatinized starch, hydroxypropylmethycellulose (HPMC), hydroxypropylmethycellulose phthalate, hydroxypropylmethylcellulose acetate stearate (HPMCAS), sucrose, xylitol, lactitol, mannitol, sorbitol, sodium chloride, polyethylene glycol, and the like.

In order to release the compound of ibrutinib and/or an anticancer agent, from a solid dosage form matrix as efficiently as possible, disintegrants are often used in the formulation, especially when the dosage forms are compressed with binder. Disintegrants help rupturing the dosage form matrix by swelling or capillary action when moisture is absorbed into the dosage form. Suitable disintegrants for use in the solid dosage forms described herein include, but are not limited to, natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, AvicerPH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

Binders impart cohesiveness to solid oral dosage form formulations: for powder filled capsule formulation, they aid in plug formation that can be filled into soft or hard shell capsules and for tablet formulation, they ensure the tablet remaining intact after compression and help assure blend uniformity prior to a compression or fill step. Materials suitable for use as binders in the solid dosage forms described herein include, but are not limited to, carboxymethylcellulose, methylcellulose (e.g., Methocel®), hydroxypropylmethylcellulose (e.g. Hypromellose USP Pharmacoat-603, hydroxypropylmethylcellulose acetate stearate (Aqoate HS-LF and HS), hydroxyethylcellulose, hydroxypropylcellulose (e.g., Klucel®), ethylcellulose (e.g., Ethocel®), and microcrystalline cellulose (e.g., Avicel®), microcrystalline dextrose, amylose, magnesium aluminum silicate, polysaccharide acids, bentonites, gelatin, polyvinylpyrrolidone/vinyl acetate copolymer, crospovidone, povidone, starch, pregelatinized starch, tragacanth, dextrin, a sugar, such as sucrose (e.g., Dipac®), glucose, dextrose, molasses, mannitol, sorbitol, xylitol (e.g., Xylitab®), lactose, a natural or synthetic gum such as acacia, tragacanth, ghatti gum, mucilage of isapol husks, starch, polyvinylpyrrolidone (e.g., Povidone® CL, Kollidon® CL, Polyplasdone® XL-10, and Povidone® K-12), larch arabogalactan, Veegum®, polyethylene glycol, waxes, sodium alginate, and the like.

In general, binder levels of 20-70% are used in powder-filled gelatin capsule formulations. Binder usage level in tablet formulations varies whether direct compression, wet granulation, roller compaction, or usage of other excipients such as fillers which itself can act as moderate binder. Formulators skilled in art can determine the binder level for the formulations, but binder usage level of up to 70% in tablet formulations is common.

Suitable lubricants or glidants for use in the solid dosage forms described herein include, but are not limited to, stearic acid, calcium hydroxide, talc, corn starch, sodium stearyl fumerate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™, PEG 4000, PEG 5000, PEG 6000, propylene glycol, sodium oleate, glyceryl behenate, glyceryl palmitostearate, glyceryl benzoate, magnesium or sodium lauryl sulfate, and the like.

Suitable diluents for use in the solid dosage forms described herein include, but are not limited to, sugars (including lactose, sucrose, and dextrose), polysaccharides (including dextrates and maltodextrin), polyols (including mannitol, xylitol, and sorbitol), cyclodextrins and the like.

The term “non water-soluble diluent” represents compounds typically used in the formulation of pharmaceuticals, such as calcium phosphate, calcium sulfate, starches, modified starches and microcrystalline cellulose, and microcellulose (e.g., having a density of about 0.45 g/cm³, e.g. Avicel, powdered cellulose), and talc.

Suitable wetting agents for use in the solid dosage forms described herein include, for example, oleic acid, glyceryl monostearate, sorbitan monooleate, sorbitan monolaurate, triethanolamine oleate, polyoxyethylene sorbitan monooleate, polyoxyethylene sorbitan monolaurate, quaternary ammonium compounds (e.g., Polyquat 10®), sodium oleate, sodium lauryl sulfate, magnesium stearate, sodium docusate, triacetin, vitamin E TPGS and the like.

Suitable surfactants for use in the solid dosage forms described herein include, for example, sodium lauryl sulfate, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like.

Suitable suspending agents for use in the solid dosage forms described here include, but are not limited to, polyvinylpyrrolidone, e.g., polyvinylpyrrolidone K12, polyvinylpyrrolidone K17, polyvinylpyrrolidone K25, or polyvinylpyrrolidone K30, polyethylene glycol, e.g., the polyethylene glycol can have a molecular weight of about 300 to about 6000, or about 3350 to about 4000, or about 7000 to about 5400, vinyl pyrrolidone/vinyl acetate copolymer (S630), sodium carboxymethylcellulose, methylcellulose, hydroxy-propylmethylcellulose, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Suitable antioxidants for use in the solid dosage forms described herein include, for example, e.g., butylated hydroxytoluene (BHT), sodium ascorbate, and tocopherol.

It should be appreciated that there is considerable overlap between additives used in the solid dosage forms described herein. Thus, the above-listed additives should be taken as merely exemplary, and not limiting, of the types of additives that can be included in solid dosage forms described herein. The amounts of such additives can be readily determined by one skilled in the art, according to the particular properties desired.

In other embodiments, one or more layers of the pharmaceutical formulation are plasticized. Illustratively, a plasticizer is generally a high boiling point solid or liquid. Suitable plasticizers can be added from about 0.01% to about 50% by weight (w/w) of the coating composition. Plasticizers include, but are not limited to, diethyl phthalate, citrate esters, polyethylene glycol, glycerol, acetylated glycerides, triacetin, polypropylene glycol, polyethylene glycol, triethyl citrate, dibutyl sebacate, stearic acid, stearol, stearate, and castor oil.

Compressed tablets are solid dosage forms prepared by compacting the bulk blend of the formulations described above. In various embodiments, compressed tablets which are designed to dissolve in the mouth will include one or more flavoring agents. In other embodiments, the compressed tablets will include a film surrounding the final compressed tablet. In some embodiments, the film coating can provide a delayed release of ibrutinib or the second agent, from the formulation. In other embodiments, the film coating aids in patient compliance (e.g., Opadry® coatings or sugar coating). Film coatings including Opadry® typically range from about 1% to about 3% of the tablet weight. In other embodiments, the compressed tablets include one or more excipients.

A capsule may be prepared, for example, by placing the bulk blend of the formulation of ibrutinib or the second agent, described above, inside of a capsule. In some embodiments, the formulations (non-aqueous suspensions and solutions) are placed in a soft gelatin capsule. In other embodiments, the formulations are placed in standard gelatin capsules or non-gelatin capsules such as capsules comprising HPMC. In other embodiments, the formulation is placed in a sprinkle capsule, wherein the capsule may be swallowed whole or the capsule may be opened and the contents sprinkled on food prior to eating. In some embodiments, the therapeutic dose is split into multiple (e.g., two, three, or four) capsules. In some embodiments, the entire dose of the formulation is delivered in a capsule form.

In various embodiments, the particles of ibrutinib and/or an anticancer agent, and one or more excipients are dry blended and compressed into a mass, such as a tablet, having a hardness sufficient to provide a pharmaceutical composition that substantially disintegrates within less than about 30 minutes, less than about 35 minutes, less than about 40 minutes, less than about 45 minutes, less than about 50 minutes, less than about 55 minutes, or less than about 60 minutes, after oral administration, thereby releasing the formulation into the gastrointestinal fluid.

In another aspect, dosage forms may include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Materials useful for the microencapsulation described herein include materials compatible with ibrutinib and/or an anticancer agent, which sufficiently isolate the compound of any of ibrutinib or an anticancer agent, from other non-compatible excipients. Materials compatible with compounds of any of ibrutinib or an anticancer agent, are those that delay the release of the compounds of any of ibrutinib or an anticancer agent, in vivo.

Exemplary microencapsulation materials useful for delaying the release of the formulations including compounds described herein, include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, HF-LG,HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® 512.5, Eudragit® NE30D, and Eudragit® NE 40D, cellulose acetate phthalate, sepifilms such as mixtures of HPMC and stearic acid, cyclodextrins, and mixtures of these materials.

In still other embodiments, plasticizers such as polyethylene glycols, e.g., PEG 300, PEG 400, PEG 600, PEG 1450, PEG 3350, and PEG 800, stearic acid, propylene glycol, oleic acid, and triacetin are incorporated into the microencapsulation material. In other embodiments, the microencapsulating material useful for delaying the release of the pharmaceutical compositions is from the USP or the National Formulary (NF). In yet other embodiments, the microencapsulation material is Klucel. In still other embodiments, the microencapsulation material is methocel.

Microencapsulated compounds of any of ibrutinib or an anticancer agent may be formulated by methods known by one of ordinary skill in the art. Such known methods include, e.g., spray drying processes, spinning disk-solvent processes, hot melt processes, spray chilling methods, fluidized bed, electrostatic deposition, centrifugal extrusion, rotational suspension separation, polymerization at liquid-gas or solid-gas interface, pressure extrusion, or spraying solvent extraction bath. In addition to these, several chemical techniques, e.g., complex coacervation, solvent evaporation, polymer-polymer incompatibility, interfacial polymerization in liquid media, in situ polymerization, in-liquid drying, and desolvation in liquid media could also be used. Furthermore, other methods such as roller compaction, extrusion/spheronization, coacervation, or nanoparticle coating may also be used.

In one embodiment, the particles of compounds of any of ibrutinib or an anticancer agent are microencapsulated prior to being formulated into one of the above forms. In still another embodiment, some or most of the particles are coated prior to being further formulated by using standard coating procedures, such as those described in Remington's Pharmaceutical Sciences, 20th Edition (2000).

In other embodiments, the solid dosage formulations of the compounds of any of ibrutinib and/or an anticancer agent are plasticized (coated) with one or more layers. Illustratively, a plasticizer is generally a high boiling point solid or liquid. Suitable plasticizers can be added from about 0.01% to about 50% by weight (w/w) of the coating composition. Plasticizers include, but are not limited to, diethyl phthalate, citrate esters, polyethylene glycol, glycerol, acetylated glycerides, triacetin, polypropylene glycol, polyethylene glycol, triethyl citrate, dibutyl sebacate, stearic acid, stearol, stearate, and castor oil.

In other embodiments, a powder including the formulations with a compound of any of ibrutinib and/or an anticancer agent, described herein, may be formulated to include one or more pharmaceutical excipients and flavors. Such a powder may be prepared, for example, by mixing the formulation and optional pharmaceutical excipients to form a bulk blend composition. Additional embodiments also include a suspending agent and/or a wetting agent. This bulk blend is uniformly subdivided into unit dosage packaging or multi-dosage packaging units.

In still other embodiments, effervescent powders are also prepared in accordance with the present disclosure. Effervescent salts have been used to disperse medicines in water for oral administration. Effervescent salts are granules or coarse powders containing a medicinal agent in a dry mixture, usually composed of sodium bicarbonate, citric acid and/or tartaric acid. When salts of the compositions described herein are added to water, the acids and the base react to liberate carbon dioxide gas, thereby causing “effervescence.” Examples of effervescent salts include, e.g., the following ingredients: sodium bicarbonate or a mixture of sodium bicarbonate and sodium carbonate, citric acid and/or tartaric acid. Any acid-base combination that results in the liberation of carbon dioxide can be used in place of the combination of sodium bicarbonate and citric and tartaric acids, as long as the ingredients were suitable for pharmaceutical use and result in a pH of about 6.0 or higher.

In some embodiments, the solid dosage forms described herein can be formulated as enteric coated delayed release oral dosage forms, i.e., as an oral dosage form of a pharmaceutical composition as described herein which utilizes an enteric coating to affect release in the small intestine of the gastrointestinal tract. The enteric coated dosage form may be a compressed or molded or extruded tablet/mold (coated or uncoated) containing granules, powder, pellets, beads or particles of the active ingredient and/or other composition components, which are themselves coated or uncoated. The enteric coated oral dosage form may also be a capsule (coated or uncoated) containing pellets, beads or granules of the solid carrier or the composition, which are themselves coated or uncoated.

The term “delayed release” as used herein refers to the delivery so that the release can be accomplished at some generally predictable location in the intestinal tract more distal to that which would have been accomplished if there had been no delayed release alterations. In some embodiments the method for delay of release is coating. Any coatings should be applied to a sufficient thickness such that the entire coating does not dissolve in the gastrointestinal fluids at pH below about 5, but does dissolve at pH about 5 and above. It is expected that any anionic polymer exhibiting a pH-dependent solubility profile can be used as an enteric coating in the methods and compositions described herein to achieve delivery to the lower gastrointestinal tract. In some embodiments the polymers described herein are anionic carboxylic polymers. In other embodiments, the polymers and compatible mixtures thereof, and some of their properties, include, but are not limited to:

Shellac, also called purified lac, a refined product obtained from the resinous secretion of an insect. This coating dissolves in media of pH >7;

Acrylic polymers. The performance of acrylic polymers (primarily their solubility in biological fluids) can vary based on the degree and type of substitution. Examples of suitable acrylic polymers include methacrylic acid copolymers and ammonium methacrylate copolymers. The Eudragit series E, L, S, RL, RS and NE (Rohm Pharma) are available as solubilized in organic solvent, aqueous dispersion, or dry powders. The Eudragit series RL, NE, and RS are insoluble in the gastrointestinal tract but are permeable and are used primarily for colonic targeting. The Eudragit series E dissolve in the stomach. The Eudragit series L, L-30D and S are insoluble in stomach and dissolve in the intestine;

Cellulose Derivatives. Examples of suitable cellulose derivatives are: ethyl cellulose; reaction mixtures of partial acetate esters of cellulose with phthalic anhydride. The performance can vary based on the degree and type of substitution. Cellulose acetate phthalate (CAP) dissolves in pH >6. Aquateric (FMC) is an aqueous based system and is a spray dried CAP psuedolatex with particles <1 μm. Other components in Aquateric can include pluronics, Tweens, and acetylated monoglycerides. Other suitable cellulose derivatives include: cellulose acetate trimellitate (Eastman); methylcellulose (Pharmacoat, Methocel); hydroxypropylmethyl cellulose phthalate (HPMCP); hydroxypropylmethyl cellulose succinate (HPMCS); and hydroxypropylmethylcellulose acetate succinate (e.g., AQOAT (Shin Etsu)). The performance can vary based on the degree and type of substitution. For example, HPMCP such as, HP-50, HP-55, HP-55S, HP-55F grades are suitable. The performance can vary based on the degree and type of substitution. For example, suitable grades of hydroxypropylmethylcellulose acetate succinate include, but are not limited to, AS-LG (LF), which dissolves at pH 5, AS-MG (MF), which dissolves at pH 5.5, and AS-HG (HF), which dissolves at higher pH. These polymers are offered as granules, or as fine powders for aqueous dispersions; Poly Vinyl Acetate Phthalate (PVAP). PVAP dissolves in pH >5, and it is much less permeable to water vapor and gastric fluids.

In some embodiments, the coating can, and usually does, contain a plasticizer and possibly other coating excipients such as colorants, talc, and/or magnesium stearate, which are well known in the art. Suitable plasticizers include triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, anionic carboxylic acrylic polymers usually will contain 10-25% by weight of a plasticizer, especially dibutyl phthalate, polyethylene glycol, triethyl citrate and triacetin. Conventional coating techniques such as spray or pan coating are employed to apply coatings. The coating thickness must be sufficient to ensure that the oral dosage form remains intact until the desired site of topical delivery in the intestinal tract is reached.

Colorants, detackifiers, surfactants, antifoaming agents, lubricants (e.g., carnuba wax or PEG) may be added to the coatings besides plasticizers to solubilize or disperse the coating material, and to improve coating performance and the coated product.

In other embodiments, the formulations described herein, which include ibrutinib and/or an anticancer agent, are delivered using a pulsatile dosage form. A pulsatile dosage form is capable of providing one or more immediate release pulses at predetermined time points after a controlled lag time or at specific sites. Many other types of controlled release systems known to those of ordinary skill in the art and are suitable for use with the formulations described herein. Examples of such delivery systems include, e.g., polymer-based systems, such as polylactic and polyglycolic acid, plyanhydrides and polycaprolactone; porous matrices, nonpolymer-based systems that are lipids, including sterols, such as cholesterol, cholesterol esters and fatty acids, or neutral fats, such as mono-, di- and triglycerides; hydrogel release systems; silastic systems; peptide-based systems; wax coatings, bioerodible dosage forms, compressed tablets using conventional binders and the like. See, e.g., Liberman et al., Pharmaceutical Dosage Forms, 2 Ed., Vol. 1, pp. 209-214 (1990); Singh et al., Encyclopedia of Pharmaceutical Technology, 2^{nd} Ed., pp. 751-753 (2002); U.S. Pat. Nos. 4,327,725, 4,624,848, 4,968,509, 5,461,140, 5,456,923, 5,516,527, 5,622,721, 5,686,105, 5,700,410, 5,977,175, 6,465,014 and 6,932,983.

In some embodiments, pharmaceutical formulations are provided that include particles of ibrutinib and/or an anticancer agent, described herein and at least one dispersing agent or suspending agent for oral administration to a subject. The formulations may be a powder and/or granules for suspension, and upon admixture with water, a substantially uniform suspension is obtained.

Liquid formulation dosage forms for oral administration can be aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2^{nd} Ed., pp. 754-757 (2002). In addition the liquid dosage forms may include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions can further include a crystalline inhibitor.

The aqueous suspensions and dispersions described herein can remain in a homogenous state, as defined in The USP Pharmacists' Pharmacopeia (2005 edition, chapter 905), for at least 4 hours. The homogeneity should be determined by a sampling method consistent with regard to determining homogeneity of the entire composition. In one embodiment, an aqueous suspension can be re-suspended into a homogenous suspension by physical agitation lasting less than 1 minute. In another embodiment, an aqueous suspension can be re-suspended into a homogenous suspension by physical agitation lasting less than 45 seconds. In yet another embodiment, an aqueous suspension can be re-suspended into a homogenous suspension by physical agitation lasting less than 30 seconds. In still another embodiment, no agitation is necessary to maintain a homogeneous aqueous dispersion.

Examples of disintegrating agents for use in the aqueous suspensions and dispersions include, but are not limited to, a starch, e.g., a natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose; a cross-linked starch such as sodium starch glycolate; a cross-linked polymer such as crospovidone; a cross-linked polyvinylpyrrolidone; alginate such as alginic acid or a salt of alginic acid such as sodium alginate; a clay such as Veegum® HV (magnesium aluminum silicate); a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

In some embodiments, the dispersing agents suitable for the aqueous suspensions and dispersions described herein are known in the art and include, for example, hydrophilic polymers, electrolytes, Tween® 60 or 80, PEG, polyvinylpyrrolidone (PVP; commercially known as Plasdone®), and the carbohydrate-based dispersing agents such as, for example, hydroxypropylcellulose and hydroxypropyl cellulose ethers (e.g., HPC, HPC-SL, and HPC-L), hydroxypropyl methylcellulose and hydroxypropyl methylcellulose ethers (e.g. HPMC K100, HPMC K4M, HPMC K15M, and HPMC K100M), carboxymethylcellulose sodium, methylcellulose, hydroxyethylcellulose, hydroxypropylmethyl-cellulose phthalate, hydroxypropylmethyl-cellulose acetate stearate, noncrystalline cellulose, magnesium aluminum silicate, triethanolamine, polyvinyl alcohol (PVA), polyvinylpyrrolidone/vinyl acetate copolymer (Plasdone®, e.g., S-630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)). In other embodiments, the dispersing agent is selected from a group not comprising one of the following agents: hydrophilic polymers; electrolytes; Tween® 60 or 80; PEG; polyvinylpyrrolidone (PVP); hydroxypropylcellulose and hydroxypropyl cellulose ethers (e.g., HPC, HPC-SL, and HPC-L); hydroxypropyl methylcellulose and hydroxypropyl methylcellulose ethers (e.g. HPMC K100, HPMC K4M, HPMC K15M, HPMC K100M, and Pharmacoat® USP 2910 (Shin-Etsu)); carboxymethylcellulose sodium; methylcellulose; hydroxyethylcellulose; hydroxypropylmethyl-cellulose phthalate; hydroxypropylmethyl-cellulose acetate stearate; non-crystalline cellulose; magnesium aluminum silicate; triethanolamine; polyvinyl alcohol (PVA); 4-(1,1,3,3-tetramethylbutyl)-phenol polymer with ethylene oxide and formaldehyde; poloxamers (e.g., Pluronics F68®, F88®, and F108®, which are block copolymers of ethylene oxide and propylene oxide); or poloxamines (e.g., Tetronic908®, also known as Poloxamine 908®).

Wetting agents suitable for the aqueous suspensions and dispersions described herein are known in the art and include, but are not limited to, cetyl alcohol, glycerol monostearate, polyoxyethylene sorbitan fatty acid esters (e.g., the commercially available Tweens® such as e.g., Tween 20® and Tween 80® (ICI Specialty Chemicals)), and polyethylene glycols (e.g., Carbowaxs 3350® and 1450®, and Carbopol 934® (Union Carbide)), oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, sodium docusate, triacetin, vitamin E TPGS, sodium taurocholate, simethicone, phosphotidylcholine and the like.

Suitable preservatives for the aqueous suspensions or dispersions described herein include, for example, potassium sorbate, parabens (e.g., methylparaben and propylparaben), benzoic acid and its salts, other esters of parahydroxybenzoic acid such as butylparaben, alcohols such as ethyl alcohol or benzyl alcohol, phenolic compounds such as phenol, or quaternary compounds such as benzalkonium chloride. Preservatives, as used herein, are incorporated into the dosage form at a concentration sufficient to inhibit microbial growth.

Suitable viscosity enhancing agents for the aqueous suspensions or dispersions described herein include, but are not limited to, methyl cellulose, xanthan gum, carboxymethyl cellulose, hydroxypropyl cellulose, hydroxypropylmethyl cellulose, Plasdon® S-630, carbomer, polyvinyl alcohol, alginates, acacia, chitosans and combinations thereof. The concentration of the viscosity enhancing agent will depend upon the agent selected and the viscosity desired.

Examples of sweetening agents suitable for the aqueous suspensions or dispersions described herein include, for example, acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sucralose, sorbitol, swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof. In one embodiment, the aqueous liquid dispersion can comprise a sweetening agent or flavoring agent in a concentration ranging from about 0.001% to about 1.0% the volume of the aqueous dispersion. In another embodiment, the aqueous liquid dispersion can comprise a sweetening agent or flavoring agent in a concentration ranging from about 0.005% to about 0.5% the volume of the aqueous dispersion. In yet another embodiment, the aqueous liquid dispersion can comprise a sweetening agent or flavoring agent in a concentration ranging from about 0.01% to about 1.0% the volume of the aqueous dispersion.

In addition to the additives listed above, the liquid formulations can also include inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, sodium lauryl sulfate, sodium doccusate, cholesterol, cholesterol esters, taurocholic acid, phosphotidylcholine, oils, such as cottonseed oil, groundnut oil, corn germ oil, olive oil, castor oil, and sesame oil, glycerol, tetrahydrofurfuryl alcohol, polyethylene glycols, fatty acid esters of sorbitan, or mixtures of these substances, and the like.

In some embodiments, the pharmaceutical formulations described herein can be self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase can be added just prior to administration, which ensures stability of an unstable or hydrophobic active ingredient. Thus, the SEDDS provides an effective delivery system for oral and parenteral delivery of hydrophobic active ingredients. SEDDS may provide improvements in the bioavailability of hydrophobic active ingredients. Methods of producing self-emulsifying dosage forms are known in the art and include, but are not limited to, for example, U.S. Pat. Nos. 5,858,401, 6,667,048, and 6,960,563, each of which is specifically incorporated by reference.

It is to be appreciated that there is overlap between the above-listed additives used in the aqueous dispersions or suspensions described herein, since a given additive is often classified differently by different practitioners in the field, or is commonly used for any of several different functions. Thus, the above-listed additives should be taken as merely exemplary, and not limiting, of the types of additives that can be included in formulations described herein. The amounts of such additives can be readily determined by one skilled in the art, according to the particular properties desired.

### Intranasal Formulations

Intranasal formulations are known in the art and are described in, for example, U.S. Pat. Nos. 4,476,116, 5,116,817 and 6,391,452, each of which is specifically incorporated by reference. Formulations that include ibrutinib and/or An anticancer agent, which are prepared according to these and other techniques well-known in the art are prepared as solutions in saline, employing benzyl alcohol or other suitable preservatives, fluorocarbons, and/or other solubilizing or dispersing agents known in the art. See, for example, Ansel, H. C. et al., Pharmaceutical Dosage Forms and Drug Delivery Systems, Sixth Ed. (1995). Preferably these compositions and formulations are prepared with suitable nontoxic pharmaceutically acceptable ingredients. These ingredients are known to those skilled in the preparation of nasal dosage forms and some of these can be found in REMINGTON: THE SCIENCE AND PRACTICE OF PHARMACY, 21st edition, 2005, a standard reference in the field. The choice of suitable carriers is highly dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents may also be present. The nasal dosage form should be isotonic with nasal secretions.

For administration by inhalation described herein may be in a form as an aerosol, a mist or a powder. Pharmaceutical compositions described herein are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, such as, by way of example only, gelatin for use in an inhaler or insufflator may be formulated containing a powder mix of the compound described herein and a suitable powder base such as lactose or starch.

### Buccal Formulations

Buccal formulations may be administered using a variety of formulations known in the art. For example, such formulations include, but are not limited to, U.S. Pat. Nos. 4,229,447, 4,596,795, 4,755,386, and 5,739,136, each of which is specifically incorporated by reference. In addition, the buccal dosage forms described herein can further include a bioerodible (hydrolysable) polymeric carrier that also serves to adhere the dosage form to the buccal mucosa. The buccal dosage form is fabricated so as to erode gradually over a predetermined time period, wherein the delivery is provided essentially throughout. Buccal drug delivery, as will be appreciated by those skilled in the art, avoids the disadvantages encountered with oral drug administration, e.g., slow absorption, degradation of the active agent by fluids present in the gastrointestinal tract and/or first-pass inactivation in the liver. With regard to the bioerodible (hydrolysable) polymeric carrier, it will be appreciated that virtually any such carrier can be used, so long as the desired drug release profile is not compromised, and the carrier is compatible with ibrutinib and/or An anticancer agent, and any other components that may be present in the buccal dosage unit. Generally, the polymeric carrier comprises hydrophilic (water-soluble and water-swellable) polymers that adhere to the wet surface of the buccal mucosa. Examples of polymeric carriers useful herein include acrylic acid polymers and co, e.g., those known as “carbomers” (Carbopol®, which may be obtained from B.F. Goodrich, is one such polymer). Other components may also be incorporated into the buccal dosage forms described herein include, but are not limited to, disintegrants, diluents, binders, lubricants, flavoring, colorants, preservatives, and the like. For buccal or sublingual administration, the compositions may take the form of tablets, lozenges, or gels formulated in a conventional manner.

### Transdermal Formulations

Transdermal formulations described herein may be administered using a variety of devices which have been described in the art. For example, such devices include, but are not limited to, U.S. Pat. Nos. 3,598,122, 3,598,123, 3,710,795, 3,731,683, 3,742,951, 3,814,097, 3,921,636, 3,972,995, 3,993,072, 3,993,073, 3,996,934, 4,031,894, 4,060,084, 4,069,307, 4,077,407, 4,201,211, 4,230,105, 4,292,299, 4,292,303, 5,336,168, 5,665,378, 5,837,280, 5,869,090, 6,923,983, 6,929,801 and 6,946,144, each of which is specifically incorporated by reference in its entirety.

The transdermal dosage forms described herein may incorporate certain pharmaceutically acceptable excipients which are conventional in the art. In one embodiments, the transdermal formulations described herein include at least three components: (1) a formulation of a compound of ibrutinib and An anticancer agent; (2) a penetration enhancer; and (3) an aqueous adjuvant. In addition, transdermal formulations can include additional components such as, but not limited to, gelling agents, creams and ointment bases, and the like. In some embodiments, the transdermal formulation can further include a woven or non-woven backing material to enhance absorption and prevent the removal of the transdermal formulation from the skin. In other embodiments, the transdermal formulations described herein can maintain a saturated or supersaturated state to promote diffusion into the skin.

Formulations suitable for transdermal administration of compounds described herein may employ transdermal delivery devices and transdermal delivery patches and can be lipophilic emulsions or buffered, aqueous solutions, dissolved and/or dispersed in a polymer or an adhesive. Such patches may be constructed for continuous, pulsatile, or on demand delivery of pharmaceutical agents. Still further, transdermal delivery of the compounds described herein can be accomplished by means of iontophoretic patches and the like. Additionally, transdermal patches can provide controlled delivery of ibrutinib and An anticancer agent. The rate of absorption can be slowed by using rate-controlling membranes or by trapping the compound within a polymer matrix or gel. Conversely, absorption enhancers can be used to increase absorption. An absorption enhancer or carrier can include absorbable pharmaceutically acceptable solvents to assist passage through the skin. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

### Injectable Formulations

Formulations that include a compound of ibrutinib and/or An anticancer agent, suitable for intramuscular, subcutaneous, or intravenous injection may include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants. Formulations suitable for subcutaneous injection may also contain additives such as preserving, wetting, emulsifying, and dispensing agents. Prevention of the growth of microorganisms can be ensured by various antibacterial and antifungal agents, such as parabens, chlorobutanol, phenol, sorbic acid, and the like. It may also be desirable to include isotonic agents, such as sugars, sodium chloride, and the like. Prolonged absorption of the injectable pharmaceutical form can be brought about by the use of agents delaying absorption, such as aluminum monostearate and gelatin.

For intravenous injections, compounds described herein may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art. For other parenteral injections, appropriate formulations may include aqueous or nonaqueous solutions, preferably with physiologically compatible buffers or excipients. Such excipients are generally known in the art.

Parenteral injections may involve bolus injection or continuous infusion. Formulations for injection may be presented in unit dosage form, e.g., in ampoules or in multi-dose containers, with an added preservative. The pharmaceutical composition described herein may be in a form suitable for parenteral injection as a sterile suspensions, solutions or emulsions in oily or aqueous vehicles, and may contain formulatory agents such as suspending, stabilizing and/or dispersing agents. Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Alternatively, the active ingredient may be in powder form for constitution with a suitable vehicle, e.g., sterile pyrogen-free water, before use.

### Other Formulations

In certain embodiments, delivery systems for pharmaceutical compounds may be employed, such as, for example, liposomes and emulsions. In certain embodiments, compositions provided herein can also include an mucoadhesive polymer, selected from among, for example, carboxymethylcellulose, carbomer (acrylic acid polymer), poly(methylmethacrylate), polyacrylamide, polycarbophil, acrylic acid/butyl acrylate copolymer, sodium alginate and dextran.

In some embodiments, the compounds described herein may be administered topically and can be formulated into a variety of topically administrable compositions, such as solutions, suspensions, lotions, gels, pastes, medicated sticks, balms, creams or ointments. Such pharmaceutical compounds can contain solubilizers, stabilizers, tonicity enhancing agents, buffers and preservatives.

The compounds described herein may also be formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

In some embodiments, the pharmaceutical compositions are formulated such that the amount of the covalent Btk inhibitor (e.g., an irreversible covalent Btk inhibitor, e.g., ibrutinib) in each unit dosage form is about 140 mg per.

### Kits/Article of Manufacture

Disclosed herein, in certain embodiments, are kits and articles of manufacture for use with one or more methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

For example, the container(s) include ibrutinib, optionally in a composition or in combination with an anti-CD20 therapeutic agent as disclosed herein. Such kits optionally include an identifying description or label or instructions relating to its use in the methods described herein.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In one embodiment, compositions containing a compound provided herein formulated in a compatible pharmaceutical carrier are also prepared, placed in an appropriate container, and labeled for treatment of an indicated condition.

### EXAMPLES

These examples are provided for illustrative purposes only and not to limit the scope of the claims provided herein.

### Example 1: Safety and Activity of BTK Inhibitor Ibrutinib Combined with Ofatumumab in Patients with Chronic Lymphocytic Leukemia

Chronic lymphocytic leukemia (CLL) is the most prevalent form of leukemia among adults in Western countries with increasing incidence in older individuals; median age of diagnosis is 72 years. It is estimated that 15,720 new cases of CLL will be diagnosed and 4,600 deaths due to the malignancy will occur in the US during 2014. CLL is characterized by an accumulation of malignant monoclonal B cells in the bone marrow, blood, lymph nodes, and other lymphoid tissues. Small lymphocytic lymphoma (SLL), immunophenotypically and morphologically identical to CLL, is characterized by a similar accumulation of cells without the leukemic component.

Treatment strategies for CLL have evolved from palliative approaches based on alkylating agents alone or in combination with purine analogue-based chemotherapy. More intensive approaches with the introduction of the anti-CD20 monoclonal antibody rituximab and its integration into combination chemotherapy regimens have led to long-term remissions in a significant proportion of patients. As a result, chemoimmunotherapy has become standard front-line treatment for fit patients with CLL. In general, CLL is incurable with current therapeutic regimens, and the treatment of relapsed disease remains challenging. Furthermore the presence of high-risk features such as unmutated IGHV, the chromosomal abnormality del(17)(p13.1), or transformation to high-grade lymphoma are associated with poor outcomes even with aggressive chemoimmunotherapy.

Although CLL is a heterogeneous disease, commonalities in the pathways in the development of a B cell to a malignant cell and in the leukemic cells themselves have allowed the development of targeted therapies. During the last decades, the B-cell receptor (BCR) pathway has emerged as a new therapeutic target in B-cell malignancies. Lying proximal within this pathway, Bruton's tyrosine kinase (BTK), a member of the Tec kinase family, plays a central role in the activation of downstream signaling required for survival and proliferation of malignant B cells. BTK is also critical for B-cell development and function in relation to the homing, migration, and adhesion of B cells to bone marrow or lymphoid tissues.

Ibrutinib is a first-in-class, orally administered, once-daily covalent inhibitor of BTK. In preclinical models, ibrutinib induced apoptosis and decreased survival of CLL cells, and inhibited their homing, migration, and adhesion to the tumor microenvironment. In a multicenter phase 1b/2 study, single-agent ibrutinib resulted in an investigator-assessed overall response rate (ORR) of 71% in patients with relapsed/refractory CLL, independent of the presence of high-risk clinical or genomic features. The estimated progression-free survival (PFS) at 26 months was 75%, indicating that responses to ibrutinib were durable. More recently, in the phase 3 RESONATE trial (PCYC-1112-CA), ibrutinib demonstrated a statistically significant 78% reduction in the risk of progression or death and a 56% reduction in the risk of death compared with ofatumumab in patients with relapsed/refractory CLL. Ibrutinib was approved by the FDA for treatment of patients with CLL who had received at least one prior therapy and for all patients with del(17)(p13.1) CLL.

Ofatumumab is an anti-CD20 monoclonal antibody that binds to an epitope distinct from that for rituximab. It exhibits more potent complement-dependent cytotoxicity and NK-cell antibody dependent cellular cytotoxicity (ADCC) compared with rituximab in B-cell lines including CLL cells. The efficacy of ofatumumab was demonstrated in a phase 2 study in patients with fludarabine-refractory CLL, who were also refractory to alemtuzumab or had bulky disease unsuitable for treatment with alemtuzumab. Ofatumumab was also effective in patients previously treated with rituximab. Recent studies have shown the feasibility and activity of ofatumumab in combination with chemotherapy. Ofatumumab is approved in the US for treatment of CLL refractory to fludarabine and alemtuzumab, and in combination with chlorambucil for front-line treatment of CLL where fludarabine-based treatment is inappropriate.

The rationale for combining ibrutinib and ofatumumab is based on proven single-agent activity in relapsed/refractory CLL, distinct mechanisms of action, and non-overlapping toxicities. The present study was designed to evaluate the safety, tolerability, and efficacy of 3 different fixed-dose regimens of ibrutinib in combination with ofatumumab in patients with relapsed/refractory CLL and related diseases. Because it was not known whether the initial lymphocytosis commonly observed with ibrutinib would predispose to development of ofatumumab infusion-related reactions or tumor lysis syndrome (TLS), three different dosing sequences were evaluated: ibrutinib was started either 4 weeks before (group 1), 1 day before (group 2), or 8 weeks after ofatumumab (group 3).

### Patients

Patients were enrolled between January 2011 and June 2012, and treated at The Ohio State University James Comprehensive Cancer Center after providing written informed consent. Patients were eligible if they had histologically confirmed CLL, SLL, B-cell prolymphocytic leukemia (PLL), as defined by the WHO classification of hematopoietic neoplasms, or Richter's transformation, and had an indication for treatment as defined by the 2008 revised International Workshop on Chronic Lymphocytic Leukemia (IWCLL) guidelines or required cytoreduction prior to stem cell transplant. Key eligibility criteria also included failure of ≧2 prior therapies including a nucleoside analogue (unless contraindicated); ≧10% expression of CD20 on CLL/SLL cells by flow cytometry; ECOG performance status ≦2; and adequate end-organ function.

### Study Design and Treatment Plan

This phase 1 b/2, single-center, open-label, sequential-group study was approved by the institutional review board and conducted in accordance with the principles of the Declaration of Helsinki and the International Conference on Harmonization Guidelines for Good Clinical Practice. The study was registered on ClinicalTrials.gov (NCT01217749).

At screening, patients underwent a complete history, physical examination, testing of laboratory parameters, and assessment of prognostic factors (including IGHV mutational analysis and β2-microglobulin). Pretreatment assessments included flow cytometry, bone marrow evaluation, and computed tomography (CT) scan of the chest, abdomen and pelvis. A baseline PET/CT scan was performed for patients with SLL and Richter's transformation.

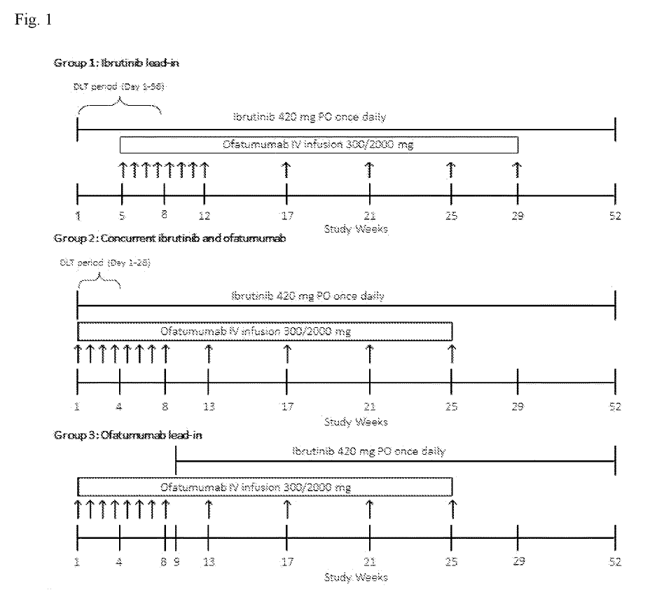

Study treatment was administered in 28-day cycles. Ibrutinib was administered orally at a dose of 420 mg once daily, and continued until disease progression or unacceptable toxicity. Ofatumumab was administered per prescribing information (300 mg for the first dose and 2000 mg for subsequent doses) intravenously for a total of 12 infusions. Patients could then continue daily ibrutinib in extension study PCYC-1103 until progression or intolerability. Patients receiving three different administration sequences were enrolled sequentially: group 1 with ibrutinib lead-in, group 2 with concomitant administration (ofatumumab on Day 1 and ibrutinib on Day 2), and group 3 with ofatumumab lead-in (FIG. 1). After the first 6 patients in group 1 demonstrated no DLT (within 56 days), enrollment was expanded to 27 patients. Expansion of enrollment in group 2 was similarly guided by safety (≦1 DLT among the first 6 patients observed for 28 days) and anti-tumor activity. Enrollment in group 2 and 3 began after enrollment was completed for groups 1 and 2, respectively.

Treatment was withheld for any grade 4 toxicity, or the individual drug was withheld for ibrutinib-related or ofatumumab-related, clinically significant, unmanageable grade 3 adverse events (AEs). Treatment was resumed after the AE returned to baseline or resolved.

Patients received premedication with acetaminophen 650 mg, cetirizine 10 mg or equivalent, and dexamethasone 20 mg intravenously prior to each dose of ofatumumab. For doses 3-12 of ofatumumab, the dexamethasone dose could be gradually reduced or discontinued if no ≧ grade 3 infusion reactions occurred during prior doses. Patients considered at risk for TLS were to be hydrated and pretreated with anti-hyperuricemics as per standard ofatumumab guidance.³⁷ Use of standard supportive care treatments, e.g. hematopoietic growth factors, was permitted.

### Assessments

Responses were assessed and reported by the investigators for patients with CLL and PLL according to IWCLL guidelines and for SLL and Richter's transformation according to the revised International Working Group criteria. A bone marrow evaluation was required to confirm a complete response (CR). An increase in peripheral blood absolute lymphocyte count (ALC) alone was not considered a treatment failure or progressive disease in the absence of other indications of disease progression. Lymphocytosis was defined as an increase in ALC of ≧50% from baseline and to an absolute value of >5×10⁹/L. Response evaluations were performed following cycles 1 and 3, and then every 3 cycles, including CT scans of the chest, abdomen, and pelvis. Safety assessments included laboratory evaluations and physical examination. Severity of AEs was defined using the Common Terminology Criteria for Adverse Events (version 4.0).

### Statistical Considerations

Safety was evaluated in all patients who received at least one dose of study drug. Efficacy was determined among evaluable patients who received at least one dose of one of the study drugs and had at least one tumor response assessment. The minimax Simon two-stage design was chosen to provide 85% power to reject the null hypothesis (for ORR of 50%) when using a 1-sided 10% alpha-level test. An interim analysis was conducted for the first 10 evaluable patients in groups 1 and 2, and the group expanded only if ≧3 patients achieved objective responses during the first 3 cycles. Efficacy was evaluated in each group separately, with no formal statistical analysis planned to compare efficacy among sequentially enrolled groups.

The primary endpoints were the number of DLTs observed among the first 6 patients enrolled in groups 1 and 2, and ORR, including CR and partial response (PR), in all groups. Secondary endpoints included incidence of AEs, (including AEs leading to ibrutinib discontinuation), ≧ grade 3 AEs, serious AEs (SAES), time to response, duration of response (DOR), PFS, and hematologic improvement. Kaplan-Meier methodology was used to estimate DOR and PFS; descriptive statistics were used for analysis of all other endpoints. Among patients with baseline cytopenias, hematologic improvement rate was evaluated as a secondary endpoint.

### Results

### Patients

A total of 71 patients were enrolled. Baseline characteristics varied among the 3 sequentially-enrolled groups (Table 1). Sixty-five patients (92%) had CLL, 1 (1%) SLL, 2 (3%) PLL, and 3 (4%) Richter's transformation. Median age was 64 years (range, 48-85 years) (Table 1). The majority (61%) had high-risk disease stage (Rai stage III or IV); 75% had bulky lymph nodes (≧5 cm), 44% had del(17)(p13.1), and 31% had del(11)(q22.3). Baseline cytopenia was present in 70% of patients. Patients had received a median of 3 prior therapies (range, 2-13) that typically included an alkylating agent, purine analogue, and rituximab (Table 2).

All 71 patients received study treatment, 68 (96%) were evaluable for response, and 66 (93%) of them had CLL/SLL. Reasons for treatment discontinuation are shown in Table 3. Nine patients (13%) discontinued treatment due to progressive disease including 4 patients in Group 3 who had progressive disease during ofatumumab monotherapy; 7 (10%) discontinued due to AEs. Fifty-four patients (76%) continued ibrutinib treatment in a long-term extension study and 2 (3%) received allogeneic stem cell transplant. The median duration of ibrutinib therapy until transition to the long-term extension study was 15.8 months in group 1, 11.3 months in group 2, and 9.2 months in group 3 (Table 3), which reflects the sequential-group design with group 1 dosed earlier than groups 2 and 3 and modification of the long-term extension study that allowed earlier enrollment for the later group. The median duration of ofatumumab treatment was similar for all 3 groups (median of 5.6 months).

### Safety

No DLTs occurred. The most common treatment-emergent AEs were diarrhea (70%), infusion-related reactions (45%), peripheral sensory neuropathy (44%) and stomatitis (38%) (Table 4). These AEs were mostly grade 1 or 2. Overall, 45 patients (63%) had ≧ grade 3 AEs, the most common events being neutropenia (24%), pneumonia (17%), and diarrhea (7%). Only 1 grade 3 infusion reaction was reported in all 71 patients. Eight patients (11%) had AEs that led to ibrutinib discontinuation, 3 patients each in groups 1 and 2, and 2 patients in group 3.

Overall, 31 patients (44%) had SAEs, including 12 (44%) in group 1, 9 (45%) in group 2, and 10 (42%) in group 3. The most common SAEs included pneumonia (16%) and atrial fibrillation (6%). Seven patients (10%) had protocol-defined major bleeding events. Seven patients experienced a major hemorrhagic event, one of which (subdural hematoma) had a fatal outcome. Four of the 7 major hemorrhages were post-procedural; 2 of the 4 procedures occurred without a prior ibrutinib dose hold that is currently recommended. The 7 events were grade 3 hemothorax (after thoracentesis for symptomatic, recurrent pleural effusion, n=1), grade 3 post-procedural hemorrhage (sinus surgery, n=1, bone marrow biopsy, n=1), grade 3 hematoma (in the knee [Baker's cyst], post-surgical; n=1), grade 2 gastrointestinal hemorrhage (due to gastric ulcer; n=1), grade 3 gastrointestinal hemorrhage (due to esophageal varices hemorrhage in a patient with ongoing medical history of esophageal varices; n=1), and subdural hematoma (n=1). The patient who experienced a subdural hematoma was taking warfarin and enoxaparin as thromboprophylaxis during the study due to a recent history of deep vein thrombosis. Other fatal non-PD events within 30 days of the last dose of study treatment occurred in 6 patients including pneumonia (n=2), organizing pneumonia (n=1), cardiorespiratory arrest (n=1), ischemic stroke (n=1), and sepsis (n=1).

### Efficacy

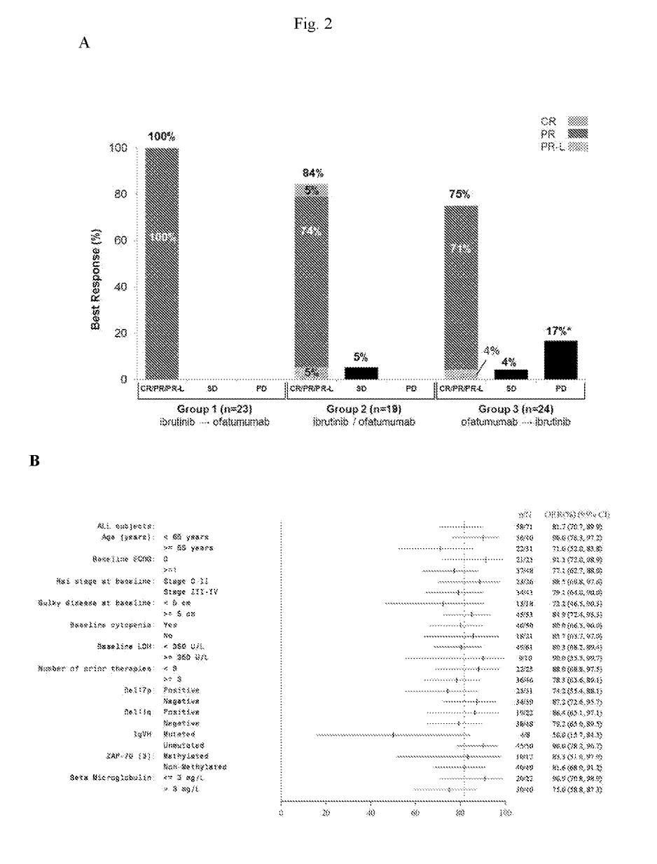

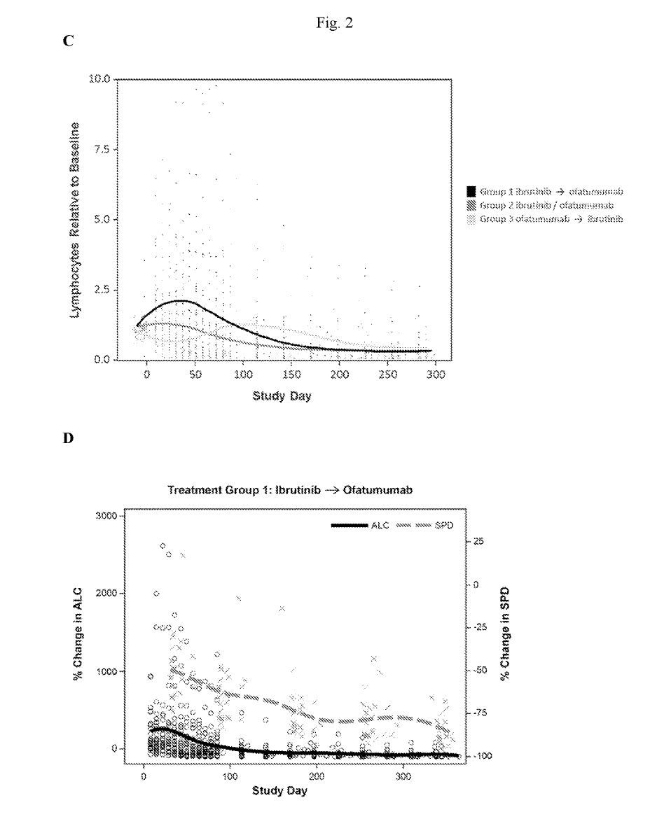

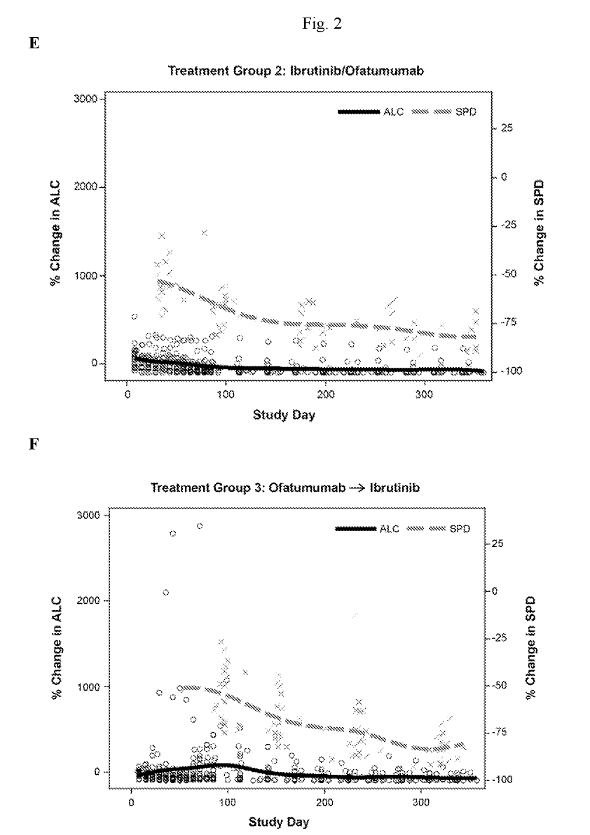

The ORR among patients with CLL/SLL was 100% (95% CI: 85.2%-100%) in group 1, 78.9% (95% CI: 54.4%-93.9%) in group 2, and 70.8% (95% CI: 48.9%-87.4%) in group 3. Among all CLL/SLL patients, the ORR was 83.3% (95% CI: 72.1%-91.4%). One patient (1.5%) achieved a CR and 54 patients (81.8%) achieved a PR. Two additional patients (3%) had a PR with lymphocytosis (PR-L). Best response among patients with CLL/SLL is shown in FIG. 2A. Both PLL patients responded to treatment with best responses of CR and PR, with DORs of 9.2+ and 11.3+ months, respectively, continued on ibrutinib in the long-term extension study, and were in response at the time of this report. Two of the patients with Richter's transformation had stable disease for 471 and 137 days before developing disease progression; 1 other patient with Richter's transformation had a best response of PR (DOR 4.6 months) before developing disease progression on Day 168. Four patients (17%) in group 3 progressed on ofatumumab monotherapy before initiating ibrutinib. The presence of high-risk features did not appear to mitigate efficacy. ORR was 71% in patients ≧65 years, 79% in Rai stage III/IV, 85% in bulky disease (≧5 cm lymph nodes), 90% in unmutated IGHV, 87% in del(17)(p13.1), and 75% in patients with β2-microglobulin >3 mg/L (FIG. 2B).

Overall, 37 of 70 patients (53%) developed lymphocytosis, including 17 (63%) in group 1, 11 (55%) in group 2, and 9 (39%) in group 3. The median percentage change in ALC over time is depicted in FIG. 2C. The median time to peak ALC was 3.1 weeks in group 1, 1.1 weeks in group 2, and 13.1 weeks in group 3, reflecting the dosing schedule. At the time of analysis, lymphocytosis resolved in all patients in group 1 within a median time to resolution of 12.1 weeks; in 9 of 11 patients in group 2 (median time to resolution: 7.6 weeks); and in 6 of 9 patients in group 3 (median time to resolution: 21.1 weeks). A reduction in lymph node size was noted for all groups (FIG. 2D). As expected, concomitant lymphocytosis was more pronounced for patients in group 1 than group 2 and 3 due to the later start of ofatumumab treatment; in all groups, ALC decreased over time.

Overall, 39 of 50 patients (78%) who had baseline cytopenias showed improvement in at least one hematologic parameter. Sustained hematologic improvement (defined as ≧50% improvement above baseline values or ANC >15004/μL or hemoglobin >11 g/dL or platelets >100,0004/μL lasting ≧56 days without transfusion or use of growth factors) was seen in 12 of 20 patients (60%) with baseline neutropenia, 18 of 33 patients (55%) with anemia, and 25 of 36 patients (69%) with thrombocytopenia.

Among the 58 patients who responded to study treatment across diseases, the median time to initial response was 2.8 months (range, 1-6) in group 1, 1 month (range, 1-3.1) in group 2, and 2.8 months (range, 2.7-7.4) in group 3. Both patients who achieved a CR did so at 12.2 months. The median DOR has not yet been reached for the overall study population or individual groups. At 12 months, the estimated rate of continued response was 88.9% (95% CI: 74.3-95.4). At the end of the study, 52 responding patients (89.7%) remained alive and progression-free.

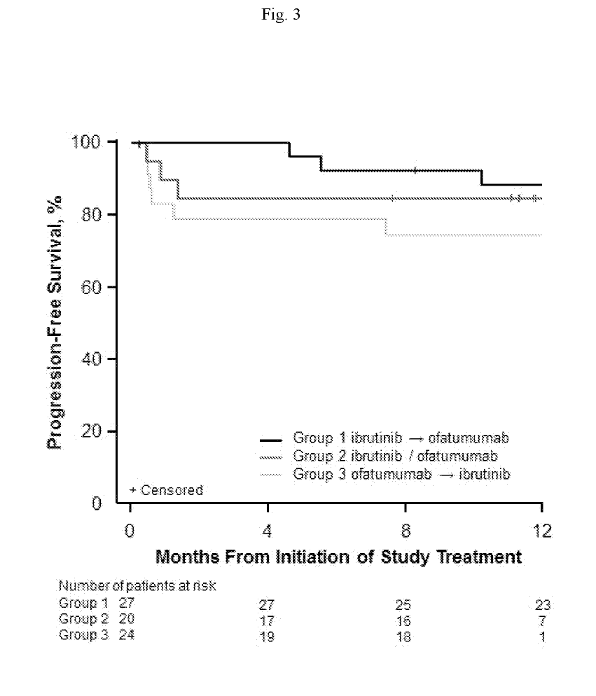

At a median time on study of 12.5 months, median PFS had not yet been reached; median follow-up was 16.4, 11.8, and 11.1 months for group 1, 2, and 3, respectively. The majority (76%) of patients continued on ibrutinib in a long-term extension study and 2 patients (2.8%) had a stem-cell transplant. The estimated 12-month PFS rate was 83.1% (95% CI: 72.1%-90%) for the entire study population, and 88.7% (95% CI: 69.0%-96.2%) in group 1, 85% (60.4%-94.9%) in group 2, and 75% (95% CI: 52.6%-87.9%) in group 3 (FIG. 3). Estimated 12-month OS was 88.6% (95% CI: 78.6%-94.2%) for the entire study population, and 92.3% (95% CI: 72.5%-98%) in group 1, 85% (95% CI: 60.4%-94.9%) in group 2, and 87.5% (95% CI: 66.1%-95.8%) in group 3.

Discussion

Herein, we demonstrate in a phase 1 b/2 study that ibrutinib and ofatumumab showed high clinical activity in patients with relapsed/refractory CLL/SLL in all 3 dose administration sequences investigated. These patients were heavily pretreated with a median of 3 prior therapies, and the majority had high-risk disease features. The response rates in all 3 groups were substantially higher than expected based on the previous experience with single-agent ofatumumab or ibrutinib at this short follow up time.^{24,31} Response to therapy was highest in the group that received ibrutinib for a month prior to ofatumumab. In contrast, response was lowest among patients receiving the lead-in with ofatumumab, possibly reflective of the diminished efficacy of this treatment compared to ibrutinib as recently demonstrated in the randomized phase 3 RESONATE™ trial in previously treated patients with CLL.²⁵ Toxicity was similar among all groups. Although direct comparison of the 3 groups is not possible based upon lack of randomization and different pre-treatment features of the patients in each group, investigators in this study have chosen to pursue the schedule of group 1 with subsequent studies combining anti-CD20 antibodies as this schedule had the highest response rate and lowest rate of infusion reactions (33%, versus 70% in group 2 and 38% in group 3). Of note, all 3 schedules allowed the majority of patients to gain benefit from the combination. Concurrent administration of ibrutinib and rituximab has been reported with similar efficacy assessed by response as observed in this trial.⁴² The investigator-determined ORR for the CLL/SLL cohort was 83.3% with an additional 3% achieving partial response with lymphocytosis, which compares favorably to regimens used historically in the treatment of relapsed and/or refractory CLL. In the PCYC-1102-CA trial, the ORR (CR and PR) was 71% for both the ibrutinib 420-mg and 840-mg cohorts, and an additional 20% and 15% of patients, respectively, had a PR-L.²⁴ Notably, the rate of PR-L with the combination (3%) is appreciably lower than in PCYC-1102-CA (14.8%). A recent 3-year update of the PCYC-1102-CA data demonstrated that 92% of patients who initially achieved a PR-L converted to conventional responses (PR/CR) with continued treatment with single-agent ibrutinib.⁴³ The median time to best response for responders was 2.8 months. In groups 1, 2, and 3, respectively, the time to best response was 3.8, 2.8, and 4.6 months. In distinction, the time to best response for relapsed or refractory patients treated with 420 mg on PCYC-1102-CA was 7.4 months.

The present study, which enrolled patients with PLL arising from CLL and also DLBCL (Richter's transformation), suggests the possibility of disease control with this combination regimen in patients with aggressive disease with the limitation that only 2 patients with PLL and 3 patients with Richter's transformation were treated. This is particularly true for PLL where 2 patients had durable remissions that persist at this time.

The high ORR of the combination was consistent across patient subgroups, even among patients with high-risk features such as del(17)(p13.1), unmutated IGHV, and elevated β2-microglobulin levels. The mutated IGHV subgroup (n=8) showed a reduced ORR (50% vs. 90%) compared with the unmutated subgroup (n=50). Two of the 8 patients with mutated IGHV were in group 3 and progressed during ofatumumab monotherapy; both initiated ibrutinib and subsequently achieved PR and PR-L. The other 6 patients had PR (n=4), PR-L (n=1), and stable disease (n=1). Examination of PFS among different genetic groups will require further follow up, as the median PFS was not reached in any subgroup analyzed. Indeed, in the PCYC-1102-CA study, only patients with del(17)(p13.1) or del(11)(q22.3) demonstrated a tendency toward progression, with PFS being the only difference that emerged over time.⁴³

Lymphocytosis is a well described pharmacodynamic class effect of BCR-inhibiting agents; with ibrutinib, this occurs by inhibition of BTK-mediated B-cell homing and adhesion to the tumor microenvironment, resulting in mobilization of leukemic cells from the lymph node compartment to the peripheral blood.^{21,23} Lymphocytosis developed in 53% of the total population of the present study, with higher rates observed when ibrutinib was started 1 month before ofatumumab (group 1: 63%), and lower rates when ofatumumab was started as monotherapy for 2 months (group 3: 39%). In previous phase 2 and 3 studies with single-agent ibrutinib, lymphocytosis developed in a higher proportion of patients (78% and 69% of patients with CLL/SLL, respectively) suggesting that the addition of an anti-CD20 monoclonal antibody may decrease the rate of lymphocytosis observed in ibrutinib-treated CLL patients.^{24,25} Temporal differences in lymphocytosis patterns across the 3 groups were expected, given the difference in the dosing sequence of ibrutinib relative to ofatumumab. Similar to reports of single-agent ibrutinib, the present study with ibrutinib and ofatumumab combined showed rapid and substantial decreases in lymph node size that occurred concomitantly with lymphocytosis.^{24,44} A recent analysis from the phase 2 trial of single-agent ibrutinib in patients with relapsed/refractory CLL showed that PR with prolonged lymphocytosis was not associated with inferior PFS outcomes compared with a traditional clinical response.⁴⁵ This finding with single-agent ibrutinib⁴⁵ along with data from the present study with the combination of ibrutinib and ofatumumab, and reported attenuation of lymphocytosis with the combination of ibrutinib and rituximab⁴⁶ raises an important question on whether additional targeting of lymphocytosis with anti-CD20 monoclonal antibodies improves long-term PFS/OS outcomes over single-agent ibrutinib therapy. This is currently being investigated in randomized studies including a phase 2 trial evaluating ibrutinib ±rituximab (NCT02007044) and the 3-arm phase 3 ALLIANCE trial comparing rituximab and bendamustine, rituximab and ibrutinib, and ibrutinib monotherapy (NCT01886872).

The most common AEs (e.g., diarrhea, infusion-related reactions, contusion/bruising, upper respiratory tract infection) observed with the combination regimens were consistent with the safety profile of the single agents in previous trials.^{24,25,31} Major bleeding events ≧ grade 3 occurred in 6 patients (8%), which is consistent with the rate reported for single-agent ibrutinib in PCYC-1102-CA, albeit slightly higher than in the recent randomized RESONATE study of single-agent ibrutinib (1%) versus ofatumumab (1.6%).^{24,25} Peripheral sensory neuropathy was reported at a relatively high rate (42%) in the present study. However, most cases were grade 1 or 2, and only 2 patients had grade 3 events. Peripheral sensory neuropathy was not a frequent AE in earlier single-agent studies with ibrutinib.^{24,25} However, in the randomized RESONATE study, peripheral sensory neuropathy (grade 1 and 2) was noted at a higher rate (13%) with ofatumumab compared with ibrutinib (4%).²⁵ These results suggest that while peripheral neuropathy may be associated with ofatumumab therapy,²⁵ it may be exacerbated in combination with ibrutinib. Peripheral neuropathy was not commonly noted in other trials investigating anti-CD20 antibodies and ibrutinib (9% of patients treated with ibrutinib in combination with rituximab).⁴² Generally, this complication did not limit the ability to administer either therapy as part of the current trial.

TABLE 1
Baseline demographic and clinical characteristics

| | Group 1 | Group 2 | Group 3 | |
|---|---|---|---|---|
| | Ibrutinib → | Ibrutinib/ | Ofatumumab → | |
| | ofatumumab | ofatumumab | ibrutinib | All patients |
| Characteristic | (N = 27) | (N = 20) | (N = 24) | (N = 71) |
| Median age (range), years | 66 (51-85) | 63 (48-75) | 63 (50-71) | 64 (48-85) |
| ≧65 years | 14 (52) | 8 (40) | 9 (38) | 31 (44) |
| ≧70 years | 12 (44) | 4 (20) | 1 (4) | 17 (24) |
| Diagnosis, n (%) | | | | |
| CLL | 22 (82) | 19 (95) | 24 (100) | 65 (92) |
| SLL | 1 (4) | 0 (0) | 0 (0) | 1 (1) |
| PLL | 1 (4) | 1 (5) | 0 (0) | 2 (3) |
| Richter's | 3 (11) | 0 (0) | 0 (0) | 3 (4) |
| transformation | | | | |
| ECOG performance status, n | | | | |
| (%) | | | | |
| 0 | 10 (37) | 8 (40) | 5 (21) | 23 (32) |
| 1 | 16 (59) | 11 (55) | 15 (63) | 42 (59) |
| 2 | 1 (4) | 1 (5) | 4 (17) | 6 (9) |
| Bulky disease, n (%) | | | | |
| Lymph nodes ≧5 cm | 19 (70) | 13 (65) | 21 (88) | 53 (75) |
| Lymph nodes ≧10 cm | 3 (11) | 3 (15) | 5 (21) | 11 (16) |
| Rai risk classification,* n (%) | | | | |
| Low risk | 1 (4) | 0 (0) | 1 (4) | 2 (3) |
| Intermediate risk | 11 (41) | 5 (25) | 8 (33) | 24 (34) |
| High risk | 14 (52) | 14 (70) | 15 (63) | 43 (61) |
| Not reported | 1 (4) | 1 (5) | (0) | 2 (3) |
| Cytopenia at baseline, n (%) | | | | |
| ANC ≦1500/μL | 5 (19) | 6 (30) | 9 (38) | 20 (28) |
| Hemoglobin ≦11 g/dL | 11 (41) | 12 (60) | 10 (42) | 33 (47) |
| Platelets ≦100,000/μL | 13 (48) | 12 (60) | 11 (46) | 36 (51) |
| Prognostic factors, n (%) | | | | |
| Unmutated IGHV | 20 (74) | 13 (65) | 17 (71) | 50 (70) |
| Del17(17)(p13.1) | 10 (37) | 9 (45) | 12 (50) | 31 (44) |
| Del(11)(q22.3) | 9 (33) | 6 (30) | 7 (29) | 22 (31) |
| β2-microglobulin >3 mg/L | 15 (56) | 12 (60) | 13 (54) | 40 (56) |

ANC = absolute neutrophil count;
ECOG = Eastern Cooperative Oncology Group;
IGHV = immunoglobulin heavy chain variable region
*Low risk, stage 0; intermediate risk, stage I or II; high risk, stage III or IV.

TABLE 2
Prior systemic therapy

| | Group 1 | Group 2 | Group 3 | |
|---|---|---|---|---|
| | Ibrutinib → | Ibrutinib/ | Ofatumumab → | |
| | ofatumumab | ofatumumab | ibrutinib | All patients |
| | (N = 27) | (N = 20) | (N = 24) | (N = 71) |
| Median number (range) of prior | 3 (2-10) | 3 (2-13) | 4 (2-12) | 3 (2-13) |
| systemic therapies | | | | |
| Types of prior systemic therapy, n | | | | |
| (%) | | | | |
| Immunotherapy | | | | |
| Antibody therapy | 27 (100) | 20 (100) | 24 (100) | 71 (100) |
| Rituximab | 26 (96) | 20 (100) | 24 (100) | 70 (99) |
| Alemtuzumab | 4 (15) | 1 (5) | 4 (17) | 9 (13) |
| Corticosteroids | 15 (56) | 10 (50) | 14 (58) | 39 (55) |
| Chemotherapy | | | | |
| Purine analogues | 24 (89) | 20 (100) | 21 (88) | 65 (92) |
| Alkylating agents | 20 (74) | 9 (45) | 14 (58) | 43 (61) |
| Anthracyclines | 6 (22) | 2 (10) | 2 (8) | 10 (14) |
| Other therapy | | | | |
| Small molecule | 9 (33) | 5 (25) | 6 (25) | 20 (28) |
| Immunomodulating agent | 8 (30) | 4 (20) | 5 (21) | 17 (24) |

TABLE 3
Patient disposition and study treatment exposure

| | Group 1 | Group 2 | Group 3 |
|---|---|---|---|
| | Ibrutinib → | Ibrutinib/ | Ofatumumab → |
| | ofatumumab | ofatumumab | ibrutinib |
| | (N = 27) | (N = 20) | (N = 24) |
| Received study treatment, n (%) | 27 (100) | 20 (100) | 24 (100) |
| Enrolled into extension study, n | 19 (70) | 16 (80) | 19 (79)* |
| (%)* | | | |
| Discontinued treatment, n (%) | | | |
| Adverse event | 2 (7) | 3 (15) | 2 (8) |
| Progressive disease | 4 (15) | 0 (0) | 5 (21) |
| Underwent stem cell | 1 (4) | 1 (5) | 0 (0) |
| transplant | | | |
| Noncompliance with | 1 (4) | 0 (0) | 0 (0) |
| study drug | | | |
| Median duration of treatment with | 15.8 (4.5-19.5) | 11.3 (0.4-14.9) | 9.2 (0.7-11.6) |
| ibrutinib (range), months | | | |
| Median duration of treatment with | 5.6 (1.4-9.6) | 5.6 (0-7.4) | 5.6 (0-8.8) |
| ofatumumab (range), months | | | |
| Median number of infusions with | 12 (7-12) | 10.5 (1-12) | 12 (1-12) |
| ofatumumab (range) | | | |

*Includes 2 patients in group 3 who discontinued ofatumumab during the ofatumumab lead-in period due to disease progression, then started on ibrutinib monotherapy and later enrolled into the extension study.

TABLE 4
Summary of adverse events

| | Group 1 | | Group 2 | | Group 3 | | | |
|---|---|---|---|---|---|---|---|---|
| | Ibrutinib → | | Ibrutinib/ | | Ofatumumab → | | | |
| | ofatumumab | | ofatumumab | | ibrutinib | | All patients | |
| | (N = 27) | | (N = 20) | | (N = 24) | | (N = 71) | |
| | | Grade ≧ | | Grade ≧ | | Grade ≧ | | Grade ≧ |
| | Any | 3 | Any | 3 | Any | 3 | Any | 3 |
| Patients with AEs leading to treatment | 3 (11) | 3 (11) | 3 (15) | 3 (15) | 2 (8) | 2 (8) | 8 (11) | 8 (11) |
| discontinuation, n (%) | | | | | | | | |
| Most common treatment-emergent AEs * | | | | | | | | |
| Diarrhea | 18 (67) | 2 (7) | 16 (80) | 3 (15) | 16 (67) | 0 (0) | 50 (70) | 5 (7) |
| Infusion-related reaction | 9 (33) | 0 (0) | 14 (70) | 1 (5) | 9 (38) | 0 (0) | 32 (45) | 1 (1) |
| Peripheral sensory neuropathy | 11 (41) | 1 (4) | 8 (40) | 0 (0) | 12 (50) | 1 (4) | 31 (44) | 2 (3) |
| Stomatitis | 11 (41) | 2 (7) | 9 (45) | 0 (0) | 7 (29) | 0 (0) | 27 (38) | 2 (3) |
| Contusion | 9 (33) | 0 (0) | 8 (40) | 0 (0) | 3 (13) | 0 (0) | 20 (28) | 0 (0) |
| Upper respiratory tract infection | 7 (26) | 0 (0) | 8 (40) | 0 (0) | 4 (17) | 0 (0) | 19 (27) | 0 (0) |
| Nausea | 5 (19) | 0 (0) | 6 (30) | 0 (0) | 6 (25) | 0 (0) | 17 (24) | 0 (0) |
| Increased tendency to bruise | 10 (37) | 0 (0) | 5 (25) | 0 (0) | 2 (8) | 0 (0) | 17 (24) | 0 (0) |
| Petechiae | 6 (22) | 0 (0) | 5 (25) | 0 (0) | 6 (25) | 0 (0) | 17 (24) | 0 (0) |
| Neutropenia^{†} | 5 (19) | 5 (19) | 7 (35) | 7 (35) | 5 (21) | 5 (21) | 17 (24) | 17 (24) |
| Muscle spasms | 10 (37) | 0 (0) | 4 (20) | 0 (0) | 2 (8) | 0 (0) | 16 (23) | 0 (0) |
| Fatigue | 13 (48) | 2 (7) | 1 (5) | 0 (0) | 1 (4) | 0 (0) | 15 (21) | 2 (3) |
| Pneumonia | 7 (26) | 5 (19) | 3 (15) | 3 (15) | 4 (17) | 4 (17) | 14 (20) | 12 (17) |
| Peripheral edema | 7 (26) | 0 (0) | 2 (10) | 0 (0) | 4 (17) | 0 (0) | 13 (18) | 0 (0) |
| Pain in extremity | 6 (22) | 0 (0) | 3 (15) | 1 (5) | 3 (13) | 0 (0) | 12 (17) | 1 (1) |
| Arthralgia | 5 (19) | 0 (0) | 2 (10) | 0 (0) | 5 (21) | 0 (0) | 12 (17) | 0 (0) |
| Dyspepsia | 5 (19) | 0 (0) | 4 (20) | 0 (0) | 2 (8) | 0 (0) | 11 (16) | 0 (0) |
| Sinusitis | 6 (22) | 0 (0) | 2 (10) | 0 (0) | 3 (13) | 0 (0) | 11 (16) | 0 (0) |
| Insomnia | 5 (19) | 0 (0) | 6 (30) | 0 (0) | 0 (0) | 0 (0) | 11 (16) | 0 (0) |
| Anemia | 7 (26) | 0 (0) | 2 (10) | 0 (0) | 2 (8) | 0 (0) | 11 (16) | 0 (0) |

* Occurring in >15% of the study population.
^{†}Includes preferred terms of neutropenia and decreased neutrophil counts.

The examples and embodiments described herein are illustrative and various modifications or changes suggested to persons skilled in the art are to be included within this disclosure. As will be appreciated by those skilled in the art, the specific components listed in the above examples may be replaced with other functionally equivalent components, e.g., diluents, binders, lubricants, fillers, and the like.

What is claimed is:

1. A combination dosing regimen for the treatment of a hematologic malignancy in a subject in need thereof comprising a first phase and a second phase, wherein the first phase is an administration of a BTK inhibitor as a single-agent treatment for a first extended period of time, and the second phase is an administration of a combination of the BTK inhibitor and an anti-CD20 therapeutic agent for a second extended period of time.

2. The combination dosing regimen of claim 1, wherein the first extended period of time is a period of up to 90 days.

3. The combination dosing regimen of claim 1, wherein the first extended period of time is a period of up to 60 days.

4. The combination dosing regimen of claim 1, wherein the first extended period of time is a period of up to 28 days.

5. The combination dosing regimen of claim 1, wherein the first extended period of time is a period of up to 14 days.

6. The combination dosing regimen of any one of the claims 1-5, wherein the second extended period of time is a period of up to 40 weeks.

7. The combination dosing regimen of any one of the claims 1-5, wherein the second extended period of time is a period of up to 35 weeks.

8. The combination dosing regimen of any one of the claims 1-5, wherein the second extended period of time is a period of up to 30 weeks.

9. The combination dosing regimen of any one of the claims 1-5, wherein the second extended period of time is a period of up to 25 weeks.

10. The combination dosing regimen of any one of the claims 1-9, wherein the combination dosing regimen is an administration of the BTK inhibitor and the anti-CD20 therapeutic agent for a period of up to 52 weeks.

11. The combination dosing regimen of any one of the claims 1-9, wherein the combination dosing regimen is an administration of the BTK inhibitor and the anti-CD20 therapeutic agent for a period of up to 37 weeks.

12. The combination dosing regimen of any one of the claims 1-9, wherein the combination dosing regimen is an administration of the BTK inhibitor and the anti-CD20 therapeutic agent for a period of up to 29 weeks.

13. The combination dosing regimen of any one of the claims 1-9, wherein the combination dosing regimen is an administration of the BTK inhibitor and the anti-CD20 therapeutic agent for a period of up to 27 weeks.

14. The combination dosing regimen of any one of the claims 1-9, wherein the combination dosing regimen is an administration of the BTK inhibitor and the anti-CD20 therapeutic agent for a period of up to 25 weeks.

15. The combination dosing regimen of any one of the claims 1-14, wherein the anti-CD20 therapeutic agent comprises ofatumumab, rituximab, obinutuzumab, ibritumomab tiuxetan, tositumomab, FBTA05, iodine I 131/tositumomab, obinutuzumab, ocaratuzumab (AME-133v), ocrelizumab, TRU-015, veltuzumab (IMMU-106), or a combination thereof.

16. The combination dosing regimen of claim 15, wherein the anti-CD20 therapeutic agent is ofatumumab.

17. The combination dosing regimen of claim 16, wherein ofatumumab is administered intravenously.

18. The combination dosing regimen of claim 16 or 17, wherein ofatumumab is administered at most 12 infusions during the course of the therapy treatment.

19. The combination dosing regimen of any one of the claims 16-18, wherein ofatumumab is administered at a dosage of about 300 mg/day to about 2000 mg/day.

20. The combination dosing regimen of any one of the claims 1-19, wherein the BTK inhibitor is ibrutinib.

21. The combination dosing regimen of claim 20, wherein ibrutinib is administered orally.

22. The combination dosing regimen of claim 20 or 21, wherein ibrutinib is administered once a day, two times per day, three times per day, four times per day, or five times per day.

23. The combination dosing regimen of claim 20 or 21, wherein ibrutinib is administered once a day.

24. The combination dosing regimen of any one of the claims 20-23, wherein ibrutinib is administered at a dosage of about 40 mg/day to about 1000 mg/day.

25. The combination dosing regimen of any one of the claims 20-23, wherein ibrutinib is administered at a dosage of about 100 mg/day to about 900 mg/day.

26. The combination dosing regimen of any one of the claims 20-23, wherein ibrutinib is administered at a dosage of about 420 mg/day to about 840 mg/day.

27. The combination dosing regimen of any one of the claims 20-23, wherein ibrutinib is administered at a dosage of about 420 mg/day.

28. The combination dosing regimen of any one of the claims 1-27, wherein the hematologic malignancy is a leukemia, a lymphoma, a myeloma, a non-Hodgkin's lymphoma, a Hodgkin's lymphoma, a T-cell malignancy, or a B-cell malignancy.

29. The combination dosing regimen of claim 28, wherein the hematologic malignancy is a B-cell malignancy.

30. The combination dosing regimen of claim 29, wherein the B-cell malignancy is chronic lymphocytic leukemia (CLL), small lymphocytic lymphoma (SLL), high risk CLL, non-CLL/SLL lymphoma, prolymphocytic leukemia (PLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

31. The combination dosing regimen of claim 29 or 30, wherein the B-cell malignancy is CLL.

32. The combination dosing regimen of claim 29 or 30, wherein the B-cell malignancy is SLL.

33. The combination dosing regimen of claim 29 or 30, wherein the B-cell malignancy is PLL.

34. The combination dosing regimen of claim 29 or 30, wherein the B-cell malignancy is DLBCL.

35. The combination dosing regimen of claim 29 or 30, wherein the B-cell malignancy is MCL.

36. The combination dosing regimen of claim 29 or 30, wherein the B-cell malignancy is Waldenström's macroglobulinemia.

37. The combination dosing regimen of any one of the claims 1-36, wherein the hematologic malignancy is a relapsed or refractory hematologic malignancy.

38. The combination dosing regimen of any one of the claims 1-37, wherein the hematologic malignancy is a metastasized hematologic malignancy.

39. The combination dosing regimen of any one of the claims 1-38, wherein the combination dosing regimen further comprises administration of an additional therapeutic agent.

40. The combination dosing regimen of claim 39, wherein the additional therapeutic agent is selected from among an analgesic, an antihistamine, a chemotherapeutic agent, or a radiation therapeutic agent.

41. The combination dosing regimen of claim 40, wherein the analgesic is acetaminophen.

42. The combination dosing regimen of claim 40, wherein the antihistamine is cetirizen.

43. The combination dosing regimen of claim 40, wherein the chemotherapeutic agent is selected from among chlorambucil, ifosfamide, doxorubicin, mesalazine, thalidomide, lenalidomide, temsirolimus, everolimus, fludarabine, fostamatinib, paclitaxel, docetaxel, dexamethasone, prednisone, CAL-101, ibritumomab, tositumomab, bortezomib, pentostatin, endostatin, or a combination thereof.

44. A combination dosing regimen for the treatment of a hematologic malignancy in a subject in need thereof comprising a first phase and a second phase, wherein the first phase is an administration of ibrutinib as a single-agent treatment for a first extended period of time, and the second phase is an administration of a combination of ibrutinib and an anti-CD20 therapeutic agent for a second extended period of time.

45. A combination dosing regimen for the treatment of chronic lymphocytic leukemia in a subject in need thereof comprising a first phase and a second phase, wherein the first phase is an administration of a BTK inhibitor as a single-agent treatment for a first extended period of time, and the second phase is an administration of a combination of the BTK inhibitor and an anti-CD20 therapeutic agent for a second extended period of time.

46. A combination dosing regimen for the treatment of chronic lymphocytic leukemia in a subject in need thereof comprising a first phase and a second phase, wherein the first phase is an administration of ibrutinib as a single-agent treatment for a first extended period of time, and the second phase is an administration of a combination of ibrutinib and an anti-CD20 therapeutic agent for a second extended period of time.

47. A method of treating a hematologic malignancy in a subject in need thereof, comprising administering to the subject a therapeutically effective amount of a combination comprising a BTK inhibitor and an anti-CD20 therapeutic agent following a combination dosing regimen wherein the combination dosing regimen comprises administering the BTK inhibitor as a single-agent over a first extended period of time as a first phase prior to administering the combination of the BTK inhibitor and the anti-CD20 therapeutic agent over a second extended period of time as a second phase.

48. The method of claim 47, wherein the first extended period of time is a period of up to 90 days as a first phase.

49. The method of claim 47, wherein the first extended period of time is a period of up to 60 days as a first phase.

50. The method of claim 47, wherein the first extended period of time is a period of up to 28 days as a first phase.

51. The method of claim 47, wherein the first extended period of time is a period of up to 14 days as a first phase.

52. The method of any one of the claims 47-51, wherein the second extended period of time is a period of up to 40 weeks.

53. The method of any one of the claims 47-51, wherein the second extended period of time is a period of up to 35 weeks.

54. The method of any one of the claims 47-51, wherein the second extended period of time is a period of up to 30 weeks.

55. The method of any one of the claims 47-51, wherein the second extended period of time is a period of up to 25 weeks.

56. The method of any one of the claims 47-55, wherein the combination dosing regimen is administered for a period of up to 52 weeks.

57. The method of any one of the claims 47-55, wherein the combination dosing regimen is administered for a period of up to 37 weeks.

58. The method of any one of the claims 47-55, wherein the combination dosing regimen is administered for a period of up to 29 weeks.

59. The method of any one of the claims 47-55, wherein the combination dosing regimen is administered for a period of up to 27 weeks.

60. The method of any one of the claims 47-55, wherein the combination dosing regimen is administered for a period of up to 25 weeks.

61. The method of any one of the claims 47-60, wherein the anti-CD20 therapeutic agent comprises ofatumumab, rituximab, obinutuzumab, ibritumomab tiuxetan, tositumomab, FBTA05, iodine I 131/tositumomab, obinutuzumab, ocaratuzumab (AME-133v), ocrelizumab, TRU-015, veltuzumab (IMMU-106), or a combination thereof.

62. The method of claim 61, wherein the anti-CD20 therapeutic agent is ofatumumab.

63. The method of claim 61 or 62, wherein ofatumumab is administered intravenously.

64. The method of any one of the claims 61-63, wherein ofatumumab is administered at most 12 infusions during the course of the therapy treatment.

65. The method of any one of the claims 61-64, wherein ofatumumab is administered at a dosage of about 300 mg/day to about 2000 mg/day.

66. The method of any one of the claims 47-65, wherein the BTK inhibitor is ibrutinib.

67. The method of claim 66, wherein ibrutinib is administered orally.

68. The method of claim 66 or 67, wherein ibrutinib is administered once a day, two times per day, three times per day, four times per day, or five times per day.

69. The method of claim 66 or 67, wherein ibrutinib is administered once a day.

70. The method of any one of the claims 66-69, wherein ibrutinib is administered at a dosage of about 40 mg/day to about 1000 mg/day.

71. The method of any one of the claims 66-69, wherein ibrutinib is administered at a dosage of about 100 mg/day to about 900 mg/day.

72. The method of any one of the claims 66-69, wherein ibrutinib is administered at a dosage of about 420 mg/day to about 840 mg/day.

73. The method of any one of the claims 66-69, wherein ibrutinib is administered at a dosage of about 420 mg/day.

74. The method of any one of the claims 47-73, wherein the hematologic malignancy is a leukemia, a lymphoma, a myeloma, a non-Hodgkin's lymphoma, a Hodgkin's lymphoma, a T-cell malignancy, or a B-cell malignancy.

75. The method of claim 74, wherein the hematologic malignancy is a B-cell malignancy.

76. The method of claim 75, wherein the B-cell malignancy is chronic lymphocytic leukemia (CLL), small lymphocytic lymphoma (SLL), high risk CLL, non-CLL/SLL lymphoma, prolymphocytic leukemia (PLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

77. The method of claim 75 or 76, wherein the B-cell malignancy is CLL.

78. The method of claim 75 or 76, wherein the B-cell malignancy is SLL.

79. The method of claim 75 or 76, wherein the B-cell malignancy is PLL.

80. The method of claim 75 or 76, wherein the B-cell malignancy is DLBCL.

81. The method of claim 75 or 76, wherein the B-cell malignancy is MCL.

82. The method of claim 75 or 76, wherein the B-cell malignancy is Waldenström's macroglobulinemia.

83. The method of any one of the claims 75-82, wherein the hematologic malignancy is a relapsed or refractory hematologic malignancy.

84. The method of any one of the claims 75-83, wherein the hematologic malignancy is a metastasized hematologic malignancy.

85. The method of any one of the claims 47-84, wherein the method further comprises administering an additional therapeutic agent.

86. The method of claim 85, wherein the additional therapeutic agent is selected from among an analgesic, an antihistamine, a chemotherapeutic agent, or a radiation therapeutic agent.

87. The method of claim 86, wherein the analgesic is acetaminophen.

88. The method of claim 86, wherein the antihistamine is cetirizen.

89. The method of claim 86, wherein the chemotherapeutic agent is selected from among chlorambucil, ifosfamide, doxorubicin, mesalazine, thalidomide, lenalidomide, temsirolimus, everolimus, fludarabine, fostamatinib, paclitaxel, docetaxel, dexamethasone, prednisone, CAL-101, ibritumomab, tositumomab, bortezomib, pentostatin, endostatin, or a combination thereof.

90. The method of any one of the claims 47-89, wherein the combination regimen leads to extension of disease remission.

91. The method of any one of the claims 47-90, wherein the combination regimen leads to a decrease of disease progression.

92. A method of treating a hematologic malignancy in a subject in need thereof, comprising administering to the subject a therapeutically effective amount of a combination comprising ibrutinib and an anti-CD20 therapeutic agent following a combination dosing regimen wherein the combination dosing regimen comprises administering ibrutinib as a single-agent over a first extended period of time as a first phase prior to administering the combination of ibrutinib and the anti-CD20 therapeutic agent over a second extended period of time as a second phase.

93. A method of treating chronic lymphocytic leukemia in a subject in need thereof, comprising administering to the subject a therapeutically effective amount of a combination comprising a BTK inhibitor and an anti-CD20 therapeutic agent following a combination dosing regimen wherein the combination dosing regimen comprises administering the BTK inhibitor as a single-agent over a first extended period of time as a first phase prior to administering the combination of the BTK inhibitor and the anti-CD20 therapeutic agent over a second extended period of time as a second phase.

94. A method of treating chronic lymphocytic leukemia in a subject in need thereof, comprising administering to the subject a therapeutically effective amount of a combination comprising ibrutinib and an anti-CD20 therapeutic agent following a combination dosing regimen wherein the combination dosing regimen comprises administering ibrutinib as a single-agent over a first extended period of time as a first phase prior to administering the combination of ibrutinib and the anti-CD20 therapeutic agent over a second extended period of time as a second phase.

95. Use of a combination of a BTK inhibitor and an anti-CD20 therapeutic agent for treating a hematological malignancy in an individual in need thereof, wherein the combination comprising the BTK inhibitor and the anti-CD20 therapeutic agent is administered following a combination dosing regimen wherein the combination dosing regimen comprises administering the BTK inhibitor as a single-agent over a first extended period of time as a first phase prior to administering the combination of the BTK inhibitor and the anti-CD20 therapeutic agent over a second extended period of time as a second phase.

96. The use of claim 95, wherein the BTK inhibitor is ibrutinib.

97. The use of any one of claims 95-96, wherein the anti-CD20 therapeutic agent comprises ofatumumab, rituximab, obinutuzumab, ibritumomab tiuxetan, tositumomab, FBTA05, iodine I 131/tositumomab, obinutuzumab, ocaratuzumab (AME-133v), ocrelizumab, TRU-015, veltuzumab (IMMU-106), or a combination thereof.

98. The use of any one of claims 95-97, wherein the hematological malignancy is a B-cell malignancy.

99. The use of claim 98, wherein the B-cell malignancy is chronic lymphocytic leukemia (CLL), small lymphocytic lymphoma (SLL), high risk CLL, non-CLL/SLL lymphoma, prolymphocytic leukemia (PLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

100. The use of claim 98, wherein the B-cell malignancy is CLL.

101. The use of claim 98, wherein the B-cell malignancy is SLL.

102. The use of claim 98, wherein the B-cell malignancy is PLL.

103. The use of claim 98, wherein the B-cell malignancy is DLBCL.

104. The use of claim 98, wherein the B-cell malignancy is MCL.

105. The use of claim 98, wherein B-cell malignancy is Waldenström's macroglobulinemia.

106. The use of any one of claims 95-105, wherein the hematological malignancy is a relapsed or refractory hematological malignancy.

107. The use of any one of claims 95-105, wherein the hematological malignancy is a metastasized hematological malignancy.

108. The use of any one of claims 95-105, wherein the hematological malignancy is a treatment naive hematological malignancy.

109. A pharmaceutical composition comprising:
a. a therapeutically-effective amount of ibrutinib;
b. an anti-CD20 therapeutic agent; and
c. a pharmaceutically-acceptable excipient.

110. The pharmaceutical composition of claim 109, wherein the anti-CD20 therapeutic agent is selected from ofatumumab, rituximab, obinutuzumab, ibritumomab tiuxetan, tositumomab, FBTA05, iodine I 131/tositumomab, obinutuzumab, ocaratuzumab (AME-133v), ocrelizumab, TRU-015, veltuzumab (IMMU-106), or a combination thereof.
